(19) United States
(12) Patent Application Publication
(10) Pub. No.: US 2019/0142761 A1
(43) Pub. Date: May 16, 2019

(54) AGENT DELIVERY SYSTEM

(71) Applicant: WRS Nutraceuticals Pty Ltd, Malvern, Victoria (AU)

(72) Inventor: Simon WEST, Williamstown, Victoria (AU)

(73) Assignee: WRS Nutraceuticals Pty Ltd, Malvern, Victoria (AU)

(21) Appl. No.: 16/099,112

(22) PCT Filed: May 5, 2017
(86) PCT No.: PCT/AU2017/050415

§ 371 Date: Nov. 5, 2018

(30) Foreign Application Priority Data
May 6, 2016 (AU) 2016901677

(51) Int. Cl. A61K 9/70; A61K 31/7032; A61K 9/00; A61K 9/51

(52) U.S. Cl. CPC A61K 9/7007; A61K 31/7032; A61K 8/68; A61K 9/51; A61K 9/0053

(57) ABSTRACT

The invention provides an agent delivery system comprising an agent preparation encapsulated within a membrane composition. The invention further provides a membrane composition comprising a ganglioside and lipid for encapsulating agents, and processes for preparing the membrane compositions. The invention further provides agent preparations encapsulated within the membrane compositions, including a plurality of individual particles wherein each particle contains agent preparation individually encapsulated within membrane composition. The invention also provides processes for preparing encapsulated agent preparations or individual particles of encapsulated agent preparations. The agents may be biologically active including hydrophilic, hydrophobic, small molecule or large molecule therapeutic agents. The invention further provides pharmaceutical compositions comprising encapsulated agent preparations or individual particles of encapsulated agent preparations. The invention further provides methods for delivery of the encapsulated agent preparations or individual particles of encapsulated agent preparations to a subject, including oral delivery of large molecule therapeutic agents to the lymphatic system, blood circulatory system, and/or targeted delivery to cells, tissues or organs.

### FIELD

The invention provides an agent delivery system comprising an agent preparation encapsulated within a membrane composition. The invention further provides a membrane composition comprising a ganglioside and lipid, and being useful for encapsulating an agent preparation, and methods for making the membrane compositions. The invention further provides an agent preparation encapsulated within a membrane composition, and methods for making the encapsulated agent preparation. The agents can be biologically active including hydrophilic, hydrophobic, small molecule or large molecule therapeutic agents, for example.

The invention further provides a pharmaceutical composition comprising an agent preparation encapsulated within a membrane composition. The invention further provides methods for delivery of the encapsulated agent preparation to a subject, including oral delivery of large molecule therapeutic agents to the lymphatic system, blood circulatory system, and/or targeted delivery to cells, tissues or organs.

### BACKGROUND

Delivery of agents to subjects raises many barriers and difficulties. Oral delivery of large molecule therapeutic agents through the gastrointestinal tract in therapeutic concentrations has proven difficult to achieve. There are also significant barriers and difficulties to delivering small or large molecules to intended tissue, whether by enteral or parenteral routes, so that agents can exert their physiological effect at their intended site of action and avoid problems associated with delivery to unintended sites. Many small molecule therapeutics also suffer from low bioavailability due to the first hepatic pass. Some other common drawbacks with agent delivery may relate to insolubility, susceptibility to degradation including enzymatic degradation and acidic denaturation before reaching the desired site of activity within a subject.

Solubility of therapeutic agents is often an issue for hydrophobic therapeutic agents. A common option for improving the solubility of an agent is to prepare it in amorphous form, which can be more soluble. However, such agents are often unstable and convert to their insoluble form.

Orally administered therapeutic agents may often be degraded or denatured in the digestive tract or blood stream before reaching their physical site of activity within a subject.

Options for overcoming these drawbacks include administering therapeutic agents with an absorption enhancer, mucoadhesive polymer, a nanoparticle, an enzyme inhibitor, a hydrogel or a dendrimer to increase the rate of uptake in the digestive tract or administering the therapeutic agent by injection. However, no drug delivery system has emerged with the capacity to orally deliver large molecules to the blood stream in a clinically and commercially effective way. Further, these drug delivery technologies do not mitigate degradation of therapeutic agents in the blood stream en route to their physiological site of action, which may be the result of enzymatic reactions in the blood stream or break down of the therapeutic agent in the liver.

Therapeutic agents absorbed in the gut are often directed into the hepatic portal venus system where they are delivered to the liver before being distributed throughout the body via the blood circulatory system. Thus, these agents are often degraded before reaching the blood circulatory system, which can result in the production of toxic bi-products and in some cases result in hepatotoxicity. Further, to ensure such agents reach their site of action, large quantities are administered, which can be costly and can exacerbate side effects. Further, drug compositions administered by injection (including almost all large molecule therapeutic agents) often have low patient compliance rates which can decrease treatment efficacy, often cause injection-related adverse events which can increase pain, discomfort, morbidity and mortality, and often are administered in the health care setting which can increase treatment cost.

Consequently, there is a clear need for novel and alternative delivery systems including compositions and methods that can deliver large molecule therapeutic agents by oral administration to the blood stream in therapeutic concentrations, deliver small molecule therapeutics by oral administration with effective bioavailability, or deliver large molecule and small molecule therapeutic agents to intended cells, tissues or organs by enteral or parenteral administration.

### SUMMARY

In a first aspect, there is provided an agent preparation encapsulated within a membrane composition, wherein the agent preparation comprises an agent, and the membrane composition comprises a lipid and ganglioside, the ganglioside having a lipophilic domain and a hydrophilic domain, wherein the hydrophilic domain comprises (i) a mono-, di-, tri-, or tetra-saccharide residue, and (ii) one to four sialic acid residues linked to the saccharide residue, the membrane composition having an outer surface, wherein at least a portion of the hydrophilic domain is present on the outer surface.

In a second aspect, there is provided a pharmaceutical composition comprising an agent preparation encapsulated within a membrane composition according to the first aspect, or any embodiments thereof as described herein, and a pharmaceutically acceptable excipient.

In a third aspect, there is provided an agent delivery system comprising an agent preparation encapsulated within a membrane composition according to the first aspect or a pharmaceutical composition according to the second aspect, or any embodiments thereof as described herein.

In a fourth aspect, there is provided a method for making a membrane composition comprising a lipid and ganglioside, wherein the method comprises the steps of:

(a) contacting a membrane composition source comprising a lipid and ganglioside with an alcohol solution to provide a liquid mixture comprising insoluble solids;

(b) separating the insoluble solids from the liquid mixture to obtain an alcohol solution comprising a lipid and ganglioside; and

(c) removing at least some of the alcohol from the alcohol solution to obtain the membrane composition.

In a fifth aspect, there is provided a method for making an agent preparation encapsulated within a membrane composition, wherein the agent preparation comprises an agent and the membrane composition comprises a lipid and ganglioside, the ganglioside having a lipophilic domain and a hydrophilic domain, wherein the hydrophilic domain comprises (i) a mono-, di-, tri-, or tetra-saccharide residue, and (ii) one to four sialic acid residues linked to the saccharide residue, and wherein the method comprises the steps of: a) combining the agent preparation with a liquid in which the agent preparation is immiscible or insoluble;

(b) subjecting the liquid and agent preparation to shear effective to form particles of the agent preparation in the liquid; and
(c) adding the membrane composition to the agent preparation at a time during or after the subjecting step (b) to encapsulate each particle of the agent preparation with the membrane composition wherein at least a portion of the hydrophilic domain is present on the outer surface of the membrane composition.

In a sixth aspect, there is provided a method of delivering an agent to the lymphatic system of a subject, comprising administering to a subject in need thereof the agent preparation encapsulated within a membrane composition according to the first aspect or the pharmaceutical composition according to the second aspect, or any embodiments thereof as described herein, wherein the agent preparation encapsulated within the membrane composition is internalized into the subject by transcytosis across the subject's gastrointestinal epithelial barrier.

In a seventh aspect, there is provided a method for delivering an agent to a predetermined cell, tissue or organ in a subject, comprising administering to a subject in need thereof the agent preparation encapsulated within a membrane composition according to the first aspect or the pharmaceutical composition according to the second aspect, or any embodiments thereof as described herein.

In an eighth aspect, there is provided a method for delivering an agent to a subject's neural cell, neural tissue or brain, comprising administering to a subject in need thereof the agent preparation encapsulated within a membrane composition according to the first aspect or the pharmaceutical composition according to the second aspect, or any embodiments thereof as described herein.

In a ninth aspect, there is provided a method for delivering an agent to a subject's adipocyte or adipose tissue, comprising administering to a subject in need thereof the agent preparation encapsulated within a membrane composition according to the first aspect or the pharmaceutical composition according to the second aspect, or any embodiments thereof as described herein.

In a tenth aspect, there is provided a method for delivering an agent to a subject's renal cell or kidney, comprising administering to a subject in need thereof the agent preparation encapsulated within a membrane composition according to the first aspect or the pharmaceutical composition according to the second aspect, or any embodiments thereof as described herein.

In an eleventh aspect, there is provided a membrane composition comprising a ganglioside and lipid, the ganglioside having a lipophilic domain and a hydrophilic domain, wherein the hydrophilic domain comprises (i) a mono-, di-, tri-, or tetra-saccharide residue, and (ii) one to four sialic acid residues linked to the saccharide residue, wherein the amount of the ganglioside is between about 0.01 to about 20% based on the total weight of the ganglioside and lipid.

In a twelfth aspect, there is provided use of a membrane composition for encapsulating an agent preparation comprising an agent and optionally one or more excipients, wherein the membrane composition comprises a lipid and ganglioside, the ganglioside having a lipophilic domain and a hydrophilic domain, wherein the hydrophilic domain comprises (i) a mono-, di-, tri-, or tetra-saccharide residue, and (ii) one to four sialic acid residues linked to the saccharide residue.

### BRIEF DESCRIPTION OF THE DRAWINGS

Preferred embodiments of the present disclosure will now be further described and illustrated, by way of example only, with reference to the accompanying drawings in which:

FIG. 1 is a confocal microscopy image of Caco-2 cells showing an agent delivered according to an embodiment of the present invention.

FIG. 2 is a confocal microscopy image of a cross-section of the jejunum of a rat showing an agent delivered according to an embodiment of the present invention.

FIG. 3 is a confocal microscopy image of a cross-section of the ilium of a rat showing an agent delivered according to an embodiment of the present invention.

FIG. 4a is schematic diagram showing an individual encapsulated particle comprising an agent preparation encapsulated with one layer of membrane composition.

FIG. 4b is schematic diagram showing an individual encapsulated particle comprising an agent preparation encapsulated with two layers of membrane composition, wherein the two layers are separated by an intervening hydrocarbon layer, according to an embodiment of the present invention.

FIG. 5 is a schematic diagram showing an encapsulated particle comprising a hydrophobic agent preparation encapsulated with a monolayer of membrane composition according to an embodiment of the present invention.

FIG. 6 is a schematic diagram showing an encapsulated particle comprising a hydrophilic agent preparation encapsulated with a bilayer of membrane composition according to an embodiment of the present invention.

FIG. 7 is a schematic diagram showing a method of making an encapsulated particle comprising an agent preparation encapsulated within a membrane composition according to an embodiment of the present invention.

FIG. 8 is an image of rat brown fat vertical section 4 h after feeding starch stabilized encapsulated microparticles with dissolved methylene blue dye agent according to an embodiment of the present invention (Magnification ×60).

FIG. 9 is an image of rat brown fat vertical section 4 h after feeding starch stabilized microparticles with dissolved methylene blue dye according to an embodiment of the present invention (Magnification ×10).

FIG. 10 is an image of brown fat from rats gavaged with microparticles encapsulating Nile red dye according to an embodiment of the present invention (formulation described in Example D4 herein). Red staining indicates accumulation of disrupted microparticles that have released Nile red dye, which has been distributed within the brown fat cells (Magnification ×43).

FIGS. 11 and 12 is an image of brain from rats gavaged with the microparticles according to an embodiment of the present invention (Example D6 containing methylene blue and agar) dissected and imaged with confocal microscopy. Methylene blue staining in brain tissue indicates that the microparticles transversed the intestinal villi intact through transcytosis and then transversed the blood brain barrier intact (magnification ×40 and ×60, respectively).

FIG. 13 is a confocal microscopy image of Caco-2 cells treated with microparticles according to an embodiment of the present invention (Example C3) (magnification ×40).

FIG. 14 is an image of brain from rats gavaged with the microparticles according to an embodiment of the present invention (Example D6 containing methylene blue and agar) stained using a silver-based stain and dissected and imaged with confocal microscopy (magnification ×120).

FIG. 15 is a series of confocal images demonstrating endocytosis of encapsulated microparticles into Caco-2 cells with decreasing concentrations of gangliosides: A) 100 dose, B) 50 dose, C) 20 dose, D) 10 dose, E) 5 dose, F) 1 dose of gangliosides. Encapsulated microparticles are visible as blue dots inside Caco-2 cells (see arrows).

FIG. 16 is a series of confocal images of sub-scapular fat tissue sections from Sprague Dawley rats treated with A) GM1 starch control, B) GM3 starch control, C) GM1 starch mCherry, D) GM3 starch mCherry. E) shows image J analysis of 6-8 confocal images per section of sub-scapular fat tissue sections from Sprague Dawley rats treated with GM1 starch formulations. Data presented are corrected total cryosection fluorescence on the red channel.

FIG. 17 shows representative images of A) HT-29 cells untreated and B) HT-29 cells treated with microparticles comprising agar, adalimumab and methylene blue as described in Example C4.

FIG. 18 shows a representative image of microparticles of 5-10 microns imaged on an Olympus IX83 deconvolution. Scale bar represents 10 μm.

FIG. 19 shows a representative image of microparticles imaged on an Olympus IX83 deconvolution. Scale bar represents 5 μm.

FIG. 20 shows a representative image on an Olympus IX83 deconvolution showing agglomeration of encapsulated agent preparation in the form of agglomeration of about 60-80 micron in size of individual microparticles.

FIGS. 21 & 22 shows a representative image on an Olympus IX83 deconvolution showing encapsulated agent preparation with solid gel core as individual microparticles using a scale of 5 and 10 μm respectively.

FIG. 23 shows a representative image on an Olympus IX83 deconvolution showing encapsulated agent preparation with liquid core as individual microparticles using a scale of 10 μm respectively.

FIG. 24 shows a representative image of formulation 3 from example D9.

### DETAILED DESCRIPTION

Reference will be made to the following abbreviations that are used in the following detailed description in which:

aNeu5Ac 5-acetyl-alpha-neuraminic acid

aNeu5Ac9Ac 5,9-diacetyl-alpha-neuraminic acid

bDGalp beta-D-galactopyranose

bDGalpNAc N-acetyl-beta-D-galactopyranose

Cer ceramide (general N-acylated sphingoid)

° C. Degrees Celsius

F Fahrenheit

h Hour

HLB Hydrophilic-Lipophilic Balance

Lecithin Egg yolk fat component with 40% phosphatidylcholine

μ, μm Micron, micrometre

nm Nanometre

Wt % Weight percentage

v/v Volume for volume

### Terms

The term “ganglioside” as used herein refers to any glycosphingolipid, having a lipophilic domain and a hydrophilic domain, wherein the hydrophilic domain comprises (i) a mono-, di-, tri-, or tetra-saccharide residue, and (ii) one to four sialic acid residues (siliac acid is N-acetylneuraminic acid, NANA) linked to the saccharide residue.

The term “lipid” as used herein refers to any lipid, other than a ganglioside as defined herein. The lipid may be a polar lipid, and illustrative lipids can include at least one of a sphingolipid, sterol lipid, phospholipid, and glycerol lipid.

The term “agent” includes any agent with an analytical or therapeutic useful property effective in a biological system including a prophylactic or therapeutic agent such as small molecule drugs and large molecule drugs, diagnostic agents, nutritional agents, cosmetic agents, foods or food ingredients, dyes, or combination thereof. The agent may be hydrophobic, hydrophilic, or amphipathic.

“Hydrocarbon” refers to a hydrocarbon compound and may for example include unsaturated or saturated aromatic, cyclic, straight or branched chain hydrocarbons ranging in size from one to about 20 carbon atoms, or more. For example, saturated hydrocarbons (alkanes) may be pentane, hexane, heptane, octane, nonane, or decane.

“Percutaneous” delivery refers to delivery made through the skin. For example, percutaneous delivery may be directly into an organ by needle injection through the skin.

“Subject” refers to any animal suitable for administration as described herein. The animal may be a vertebrate. The animal may be a mammal, avian, arthropod, chordate, amphibian or reptile. The animal may be a human, rat, mouse, guinea pig, monkey, pig, goat, cow, horse, dog, cat, bird and fowl.

“Hydrophilic” may be used to refer to the hydrophilicity of an agent or compound useful in the present invention, or a composition or liquid comprising the agent or compound. The hydrophilicity of an agent, compound, composition or liquid, may be described by a hydrophilic-lipophilic balance (HLB). A hydrophilic agent or compound typically has an HLB above about 10, for example between about 10 and 20.

“Hydrophobic” may be used to refer to the hydrophobicity of an agent or compound useful in the present invention, or a composition or liquid comprising the agent or compound. The hydrophobicity of an agent, compound, composition or liquid, may be described by a hydrophilic-lipophilic balance (HLB). A hydrophobic agent or compound typically has an HLB below about 10, for example between about 0 and 10.

It will be appreciated that the HLB system is routinely used to evaluate the hydrophilic nature or hydrophobic nature of both surfactant based agents and non-surfactant based agents, with hydrophilic materials having an HLB greater than about 10 and hydrophobic materials having an HLB value generally below about 10.

It will also be understood that the hydrophobic and hydrophilic properties of agents can also be expressed in terms of Log P values. The higher the value of log P, the greater the hydrophobicity and thus lipid solubility of the agent or agents. The lower the value of log P, the greater the hydrophilicity and thus water solubility of the agent or agents. A hydrophobic agent therefore typically, but is not limited to, has a Log P of least 0.2 or above, for example between 0.2 and 5. In one example, a hydrophobic agent may have a Log P of at least 1 or above. Some hydrophobic agents may also have a Log P of at least 2 or above and also those molecules having a Log P of at least 3 or above. A hydrophilic agent typically has, but is not limited to, a Log P of about zero or less, preferably less than 0.2.

It will be appreciated that Log P is the log of the octanol-water or buffer partition coefficient and can be determined by a variety of methods for those skilled in the art. For example, Log P refers to the mathematical resultant of the logarithmic base-10 function of the Partition Coefficient, P; wherein P is the relative ratio of the solubility of a compound in an organic phase relative to the solubility of the same compound in an aqueous phase. In other words, P is the ratio of the concentration of a compound in an organic phase, which is represented by “[Organic],” to the concentration of the same compound in an aqueous phase, which is represented by [Aqueous]”. Mathematically, Log P=log 10([Organic]/[Aqueous]). As such, Log P is a value which reflects the intrinsic hydrophilic and hydrophobic nature of a compound or drug and is therefore independent of the pH of the solvent and the pKa of the compound.

It will be appreciated that for the above terms “hydrophilic” and “hydrophobic”, an HLB for a non-ionic compound can be determined using the method originally described by Griffin in 1954 and an HLB for an ionic compound can be determined using the method originally described by Davies in 1957 (see Pharmaceutical Emulsions and Suspensions, 2 ed.). The HLB value determined using the Griffin formula for a non-ionic compound can be calculated using the following formula: HLB=20×Mₕ/M where Mₕ is the molecular weight of the hydrophilic domain(s) of the non-ionic compound, and M is the total molecular weight of the compound. The HLB value determined using the Davies formula (1957) for an ionic compound can be calculated using the following formula: HLB=7+Σ(Hᵢ)−n×0.475, where H, is the value of the hydrophilic group(s) which can be found in, for example, Pharmaceutical Emulsions and Suspensions, 2 ed., and n is the number of carbon atoms in the hydrophobic tail(s).

“Targeted” or “Targeting” generally refers to enhancing delivery, binding and/or accumulation of agents to, at, near or inside pre-determined cells, tissues or organs.

“Delivered” or “delivering” generally refers to transport or accumulation of agents to, at or near a site or location in the body of a subject, for example lymphatic system, blood circulatory system, blood plasma, or pre-determined cells, tissues or organs.

The phrase “pharmaceutically acceptable salt,” as used herein, refers to pharmaceutically acceptable organic or inorganic salts of an agent. Where an agent comprises at least one amino group, acid addition salts can be formed with this amino group. Illustrative acid addition salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. Where an agent comprises at least one carboxyl group, ammonium, hydroxide, carbonate or bicarbonate salts can be formed with this carboxyl group [etc.]. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion. “Pharmaceutically acceptable solvate” or “solvate” refer to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Those skilled in the art will appreciate that the disclosure herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The term “about” when used in connection with a referenced numeric indication means the referenced numeric indication plus or minus up to 10% of that referenced numeric indication. For example, the language “about 50” covers the range of 45 to 55.

The term “and/or”, e.g., “X and/or V” shall be understood to mean either “X and Y” or “X or V” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Each example of the present disclosure described herein is to be applied mutatis mutandis to each and every other example unless specifically stated otherwise. The present disclosure is not to be limited in scope by the specific examples described herein, which are intended for the purpose of exemplification only. Functionally-equivalent products, compositions and methods are clearly within the scope of the disclosure as described herein.

It will be clearly understood that, although a number of publications are referred to herein, this reference does not constitute an admission that any of these documents forms part of the common general knowledge in the art, in Australia or in any other country.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Encapsulated Agent Preparations

An agent preparation can be encapsulated within a membrane composition. Agent preparations comprise at least one agent, and optionally one or more excipients. For example, an “agent preparation” can be a composition comprising an agent and an excipient. Various embodiments of the “agent preparation” according to the present invention are described below.

A membrane composition comprises a lipid and ganglioside, the ganglioside having a lipophilic domain and a hydrophilic domain, wherein the hydrophilic domain comprises (i) a mono-, di-, tri-, or tetra-saccharide residue, and (ii) one to four sialic acid residues linked to the saccharide residue. Various embodiments of the “membrane composition” according to the present invention are described below.

An “encapsulated agent preparation” as used herein refers to the “agent preparation encapsulated within a membrane composition”. The encapsulated agent preparation is useful in compositions and methods for administration or delivery to subjects, for example, according to at least some embodiments, transcytosis of intact individually encapsulated particles into the lymphatic system, transportation into the blood circulatory system, and/or delivery to the blood circulatory system, blood plasma, or intended cells, tissues or organs.

In an embodiment, an agent preparation can be encapsulated within a membrane composition, wherein the agent preparation comprises an agent and optionally one or more excipients, and the membrane composition comprises a lipid and ganglioside, the ganglioside having a lipophilic domain and a hydrophilic domain, wherein the hydrophilic domain comprises (i) a mono-, di-, tri-, or tetra-saccharide residue, and (ii) one to four sialic acid residues linked to the saccharide residue, the membrane composition having an outer surface, wherein at least a portion of the hydrophilic domain is present on the outer surface.

The encapsulated agent preparation can be provided as a plurality of encapsulated particles. The encapsulated particles can be individually encapsulated particles. For example, the agent preparation can exist as, or be formed into agglomerated particles or individual particles. It will be appreciated that the agent preparation can comprise an agent and excipient, e.g., a composition or mixture of agent and excipient, and therefore in one embodiment, an individual particle of the agent preparation can contain a composition or mixture of both the agent and excipient within the same particle. In other words, the term “particle” refers to an individual portion (or discrete unit) of agent preparation material, which can be encapsulated by a layer of membrane composition to provide an “encapsulated particle”. The lipid and the lipophilic domain of the ganglioside of the membrane composition may exist as one or more layers that encapsulate one or more particles to provide a plurality of encapsulated particles, including a plurality of individually encapsulated particles. It will also be appreciated that an “encapsulated particle” is a particle that is encapsulated within one or more layers of the membrane composition.

The agent preparation may be provided in the form of a liquid, gel or solid. In one embodiment, the agent preparation may be provided in the form of a substantially solidified gel or a solid. In one embodiment, the agent preparation is a solid agent preparation, such as provided in the form of a solid (e.g. powder or granules) or solid mixture (e.g. powder and/or granule mixture). For example, the encapsulated agent preparation may comprise a core comprising the agent preparation according to any embodiments thereof as described herein, wherein the surface of the core comprises a coating of the membrane composition according to any embodiments thereof as described herein. The core may be a substantially solidified or solid core. For example, the encapsulated agent preparation may be provided by a membrane composition that encapsulates a solid agent preparation, or may be provided by a solid core comprising one or more agents and optionally one or more excipients wherein the solid core is coated in the membrane composition.

FIGS. 4-6 show various example embodiments of an encapsulated particle, in which the particle is formed from an agent preparation comprising one or more agents and optionally one or more excipients (e.g. oil or gelling agent), and the particle is encapsulated by one or more layers of a membrane composition comprising a ganglioside and lipid.

FIG. 4a shows one embodiment of an encapsulated particle (1) comprising the agent preparation (2), which can be a mixture of agent and gelling excipient, for example. In this example, the agent preparation (2) is encapsulated within a layer of membrane composition (3).

FIG. 4b shows another embodiment of an encapsulated particle (1) located within a hydrophilic fluidic medium (5). The encapsulated particle (1) comprises the agent preparation (2), which can be a mixture of agent and gelling excipient, for example. In this example, the agent preparation (2) is encapsulated within a first layer of membrane composition (3a) and also a second layer of membrane composition (3b). An intervening layer of hydrocarbon fluid (4) (e.g. heptane) can also exist between the first and second layers (3a and 3b) of the membrane composition. The intervening layer of hydrocarbon fluid is optional.

In an embodiment, the agent or agent preparation can be hydrophobic. For example, the agent preparation, or agent and/or excipient thereof, can have an HLB below about 9, 8, 7, 6, 5, 4, or 3, or as otherwise described herein. In various embodiments, the agent preparation, or agent and/or excipient thereof, can have an HLB between about 0 and 10, between about 0 and 8, between about 0 and 6, or between about 0 and 4. In embodiments where the agent or agent preparation is hydrophobic, the membrane composition can encapsulate the agent preparation or particles thereof, including individual particles, with a first monolayer comprising a lipid and ganglioside, the ganglioside having a lipophilic domain and a hydrophilic domain, wherein the hydrophilic domain comprises (i) a mono-, di-, tri-, or tetra-saccharide residue, and (ii) one to four sialic acid residues linked to the saccharide residue, the monolayer having an outer surface, wherein at least a portion of the hydrophilic domain of the ganglioside is present on the outer surface. One or more further layers of membrane composition can be provided as bilayers that encapsulate the first monolayer, wherein the outermost bilayer has an outer surface, and wherein at least a portion of the hydrophilic domain of the ganglioside is present on the outer surface.

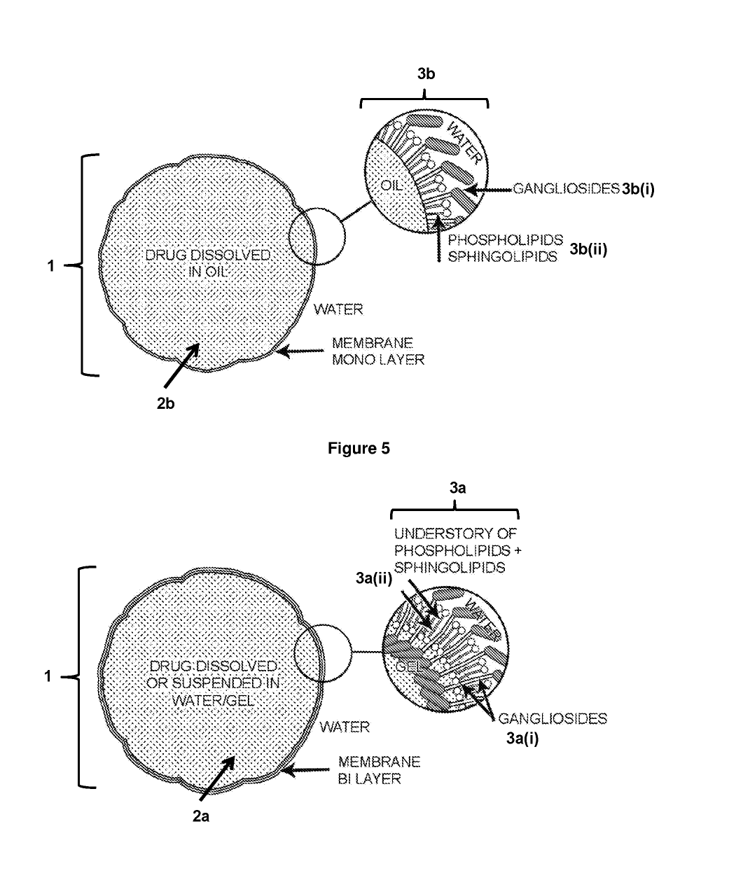

FIG. 5 shows one embodiment of an individual encapsulated particle (1) comprising an agent preparation (2b) that is hydrophobic. The hydrophobic agent preparation (2b) can comprise a hydrophobic agent or excipient, for example the excipient can be an oil containing the agent. The hydrophobic agent preparation (2b) is encapsulated by a first monolayer of the membrane composition (3b) wherein at least a portion of the hydrophilic domain (3b(i)) of the ganglioside in the monolayer is present on the outer surface. In this example the monolayer also comprises further lipids (3b(ii)) such as phospholipids and sphingolipids.

In another embodiment, the agent or agent preparation can be hydrophilic. For example, the agent preparation, or agent and/or excipient thereof, can have an HLB above about 10, 11, 12, 13, 14, or 15, or as otherwise described herein. In various embodiments, the agent preparation, or agent and/or excipient thereof, can have an HLB between about 10 and 20, between about 11 and 20, between about 12 and 20, between about 13 and 20, or between about 14 and 20. In embodiments where the agent or agent preparation is hydrophilic, the membrane composition can encapsulate the agent preparation or particles thereof, including individual particles, with a first bilayer comprising a lipid and ganglioside, the ganglioside having a lipophilic domain and a hydrophilic domain, wherein the hydrophilic domain comprises (i) a mono-, di-, tri-, or tetra-saccharide residue, and (ii) one to four sialic acid residues linked to the saccharide residue, the bilayer having an outer surface, wherein at least a portion of the hydrophilic domain of the ganglioside is present on the outer surface. One or more further layers of membrane composition can be provided as bilayers that encapsulate the first bilayer, wherein the outermost bilayer has an outer surface, and wherein at least a portion of the hydrophilic domain of the ganglioside is present on the outer surface.

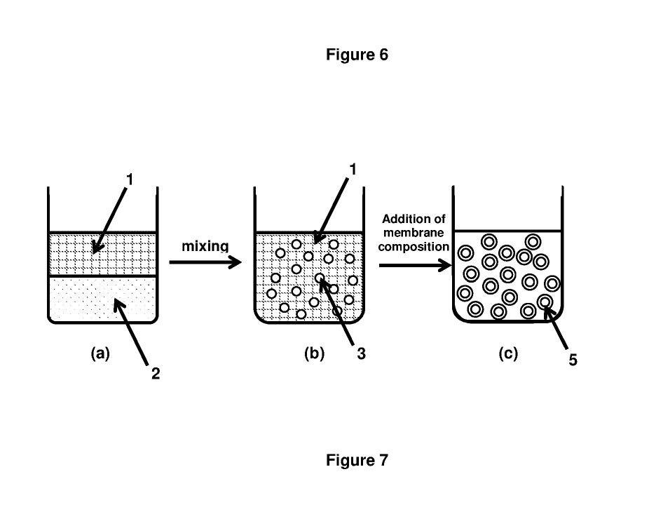

FIG. 6 shows one embodiment of an individual encapsulated particle (1) comprising an agent preparation (2a) that is hydrophilic. The hydrophilic agent preparation (2a) can comprise a hydrophilic agent or excipient, for example the excipient can be a gel containing the agent. The hydrophilic agent preparation (2a) is encapsulated by a first bilayer (3a) of the membrane composition wherein at least a portion of the hydrophilic domain (3a(i)) of the ganglioside in the monolayer is present on the outer surface. In this example, the bilayer also comprises further lipids (3a(ii)) such as phospholipids and sphingolipids.

The encapsulated particle can have a diameter ranging from about 0.001 to about 100 μm, for example less than 75 μm or less than 50 μm. In one embodiment, the encapsulated particle can have a diameter ranging from about 0.001 to about 20 μm. It will be appreciated that the diameter is that of the particle and its one or more layers of membrane composition. The size of each encapsulated particle can be provided by diameters (in μm) ranging from about 0.001 to 20, 0.01 to 15, 0.1 to 10, 0.2 to 5, or 0.4 to 4, or 0.5 to 3. The size of each encapsulated particle can be provided by diameters (in μm) of less than about 20, 15, 10, 5, 4, 3, 2, 1, 0.5, 0.1, or 0.01. The size of each encapsulated particle can be provided by diameters (in μm) of greater than about 0.001, 0.01, 0.1, 0.5, 1, or 2. It will be appreciated that a plurality of encapsulated particles may agglomerate together, which would provide an agglomeration size equivalent to the combination of the sizes of the individual encapsulated particles. For example, there may be provided a plurality of individual encapsulated particles wherein each individual encapsulated particle has a diameter ranging from about 0.001 to about 20 μm or an agglomeration of encapsulated particles wherein the agglomeration size has a diameter ranging from about 0.1 to about 500 μm. The agglomeration size may be provided by diameters (in μm) ranging from about 0.1 to 500, 1 to 400, 5 to 300, 10 to 200, 15 to 100, or 20 to 75,

In further embodiments, the particles can be nanoparticles or microparticles. It will be appreciated that the nanoparticles or microparticles according to the present disclosure as described herein are not liposomes.

The size of each encapsulated microparticle (including one or more layers of membrane composition) can be provided by diameters (in μm) ranging from about 0.1 to 10, 0.2 to 9, 0.3 to 8, 0.4 to 7, 0.5 to 6, 0.6 to 5, 0.7 to 4, or 0.8 to 3. The size of each encapsulated microparticle (including one or more layers membrane composition) can be provided by diameters (in μm) of less than about 10, 5, 4, 3, 2, or 1. The size of each encapsulated microparticle (including one or more layers membrane composition) can be provided by diameters (in μm) of greater than about 0.1, 0.2, 0.3, 0.4, 0.5, 0.6, 0.7, 0.8, 0.9, 1, or 2.

The size of each encapsulated nanoparticle (including one or more layers membrane composition) can be provided by diameters (in nm) ranging from about 1 to 500, 2 to 250, 5 to 100, 10 to 50. The size of each encapsulated nanoparticle (including one or more layers membrane composition) can be provided by diameters (in nm) of less than about 500, 250, 100, 75, 50, 25, or 10. The size of each encapsulated nanoparticle (including one or more layers membrane composition) can be provided by diameters (in nm) of greater than about 1, 2, 3, 4, 5, 10, 15, 20, 30, 40, 50, 75, or 100.

Without wishing to be bound by any theory, it is believed that the hydrophilic domain of the ganglioside of the membrane composition contributes to the surface properties of the encapsulated agent preparation, or encapsulated particles thereof, which may facilitate enhanced resistance to enzymatic degradation and transport by transcytosis as intact encapsulated particles to the lymphatic system. In some embodiments the encapsulated agent preparations, including encapsulated particles, may facilitate entry into the blood circulatory system via the lymphatic vasculature, for example entry and subsequent release into the blood plasma. Delivery via the lymphatic system bypasses the hepatic portal venus system and therefore the liver, where agents are often degraded before reaching the blood circulatory system. It is also believed that the surface properties can enable or facilitate targeted delivery to specific cells, tissues or organs. Further advantages may be provided by the selection of excipients in the agent preparations. The properties may also be modified or further enhanced by varying the membrane composition or excipients of the agent preparation, for example varying amounts, concentrations, ratios or types of gangliosides in the membrane composition compared to the amounts, concentrations, ratios or types of lipids.

### Membrane Composition

The membrane composition comprises a lipid and ganglioside, the ganglioside having a lipophilic domain and a hydrophilic domain, wherein the hydrophilic domain comprises a mono-, di-, tri-, or tetra-saccharide residue, and includes one to four sialic acid residues. The membrane composition can exist as one or more layers that encapsulate the agent preparation, or encapsulate one or more particles thereof.

The ganglioside of the membrane composition can comprise or consist of any one or more species of gangliosides as described herein. The ganglioside can provide or facilitate transcytosis of intact encapsulated particles into the lymphatic system, transportation of the intact encapsulated particles into the blood circulatory system, and/or targeted delivery of the intact encapsulated particles to specific cells, tissues or organs.

The lipid of the membrane composition can comprise or consist of any one or more lipids as described herein. The lipid may be a polar lipid, for example an amphiphilic compound such as a surfactant having a polar head group and a non-polar tail group. The lipids may facilitate support of the gangliosides in forming one or more encapsulating layers, for example a membrane layer, around the agent preparation or particle thereof. The lipid can provide a supporting understory for the gangliosides to facilitate stability of the layer comprising gangliosides.

The membrane composition can be useful to encapsulate agents for administration to a subject or for delivery to the lymphatic system of a subject by transcytosis across the subject's gastrointestinal epithelial barrier. The membrane composition may also be useful, for example, to encapsulate a microscale or nanoscale medical device.

The membrane composition may be obtained from one or more natural extracts, such as an extract from milk or nerve tissue. The extract from milk can be an extract from butter milk powder or other milk product comprising milk fat globule membrane (MFGM). The natural extract from MFGM provides a membrane composition comprising, and in some embodiments enriched in, a GD3 ganglioside and GM3 ganglioside among other gangliosides, and the extract from nerve tissue of an animal such as a mammal or non-mammal such as felines, bovines, pigs, horses and fish, can for example provide a membrane composition comprising, and in some embodiments enriched in, a GM1 ganglioside. In one embodiment, the membrane composition is obtainable from MFGM. Useful processes for obtaining suitable extracts from such natural sources are described in further detail below. It will be appreciated that the membrane compositions may also be made synthetically or semi-synthetically.

The amount of ganglioside in the membrane composition can range from about 0.01 to about 20 weight % of the total weight of the ganglioside and lipid. For example, the amount of ganglioside in the membrane composition (in weight %), based on the total weight % of the ganglioside and lipid, can range from about 0.01 to about 10, about 0.02 to about 5, about 0.04 to about 4, about 0.05 to about 3, about 0.06 to about 2, about 0.07 to about 1, 0.08 to about 0.9, about 0.09 to about 0.9, or about 0.1 to about 0.5. The amount of ganglioside in the membrane composition (in weight %), based on the total weight % of the ganglioside and lipid, can be at least about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.25, 0.3, 0.35, 0.4, 0.45, or 0.5. The amount of ganglioside in the membrane composition (in weight %), based on the total weight % of the ganglioside and lipid, can be less than about 20, 15, 10, 9, 8, 7, 6, 5, 4, 3, 2, 1, 0.9, 0.8, 0.7, 0.6, 0.5, 0.4, 0.3, 0.2, or 0.1.

The amount of lipid in the membrane composition can range from about 0.05 to 10 weight % of the total weight of the ganglioside and lipid. The amount of lipid in the membrane composition can range from about 0.1% to 5% or 0.5 to 3% of the total weight of the ganglioside and lipid. The amount of lipid in the membrane composition can be at least about 0.05, 0.1, 0.2, 0.3, 0.4, 0.5, 0.6, 0.7, 0.8, 0.9, 1, 2, 3, or 4 weight % of the total weight of the ganglioside and lipid. The amount of lipid in the membrane composition can be less than about 10, 9, 8, 7, 6, 5, 4, 3, 2, or 1 weight % of the total weight of the ganglioside and lipid. The ratio of ganglioside:lipid (by weight) in the membrane composition can range from about 1:10,000 to 1:3. For example, the ratio of ganglioside:lipid (by weight) in the membrane composition can range from about 1:5000 to 1:20, 1:4000 to 1:50, 1:3000 to 1:75, 1:2000 to 1:100, or 1:1000 to 1:200. The ratio of ganglioside:lipid (by weight) in the membrane composition can be at least about 1:5000, 1:4000, 1:3000, 1:2000, 1:1000, 1:500, 1:200, 1:100, or 1:50. The ratio of ganglioside:lipid (by weight) in the membrane composition can be less than about 1:3, 1:4, 1:5, 1:6, 1:7, 1:8, 1:9, 1:10, 1:20, 1:30, 1:40, 1:50, 1:75, 1:100, 1:125, 1:150, 1:175, 1:200, 1:250, 1:300, 1:400, or 1:500.

In one embodiment, the lipid in the membrane composition comprises phospholipids and cholesterol. The phospholipids can comprise, for example, sphingomyelin, phosphatidyl choline, and phosphatidyl ethanol. These phospholipids in this example may be present in various ratios, such as about 4:about 2:about 1 ratio, respectively. The cholesterol can also be present in various amounts, for example between about 1 to about 10 weight % of the total weight of the ganglioside and lipid. It will also be appreciated that the membrane composition for this embodiment can also provide various amounts of ganglioside as described above, for example about 0.05 to about 1 weight % of the total weight of the ganglioside and lipid.

Gangliosides

The membrane composition comprises a ganglioside. Gangliosides are amphipathic molecules having a lipophilic domain and a hydrophilic domain, wherein the hydrophilic domain comprises (i) a mono-, di-, tri-, or tetra-saccharide residue, and (ii) one to four sialic acid (N-acetylneuraminic acid (“NANA”)) residues linked to the saccharide residue. Such gives rise to the different known gangliosides. It will be appreciated that the membrane composition may comprise any one or more of the various known gangliosides, or be enriched in any one or more of the various known gangliosides.

Useful gangliosides have one or more N-acetylneuraminic acid (“NANA”) groups. Examples of gangliosides having one NANA group are GM1, GM2, and GM3. Examples of gangliosides having two NANA groups are GD1a, GD1b, GD2, and GD3. Examples of gangliosides having three NANA groups are GT1b and GT3. An example of a ganglioside having four NANA groups is GQ1.

The chemical name for GM1 is (2S,4S,5R,6R)-5-acetamido-2-[(2S,3R,4R,5S,6R)-5-[(2S,3R,4R,5R,6R)-3-acetamido-5-hydroxy-6-(hydroxymethyl)-4-[(2R,3R,4S,5R,6R)-3,4,5-trihydroxy-6-(hydroxymethyl)oxan-2-yl]oxyoxan-2-yl]oxy-2-[(2R,3S,4R,5R,6R)-4,5-dihydroxy-6-[(E,2R,3S)-3-hydroxy-2-(icosanoylamino)icos-4-enoxy]-2-(hydroxymethyl)oxan-3-yl]oxy-3-hydroxy-6-(hydroxymethyl)oxan-4-yl]oxy-4-hydroxy-6-[(1R,2R)-1,2,3-trihydroxypropyl]oxane-2-carboxylic acid. GM1 is also known as monosialotetrahexosylganglioside. The chemical structure of GM1 is provided as follows:

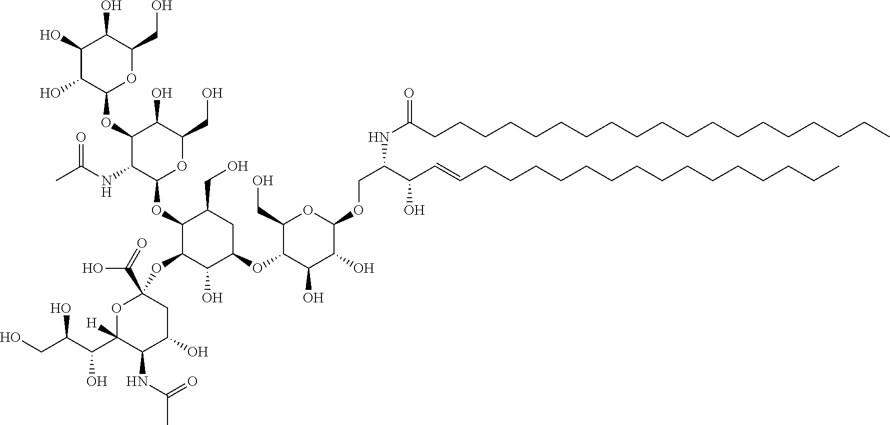 [SMILES: CCCCCCCCCCCCCCC/C=C/[C@H](O)[C@H](CO[C@@H]1O[C@H](CO)[C@@H](O[C@@H]2C[C@H](CO)[C@H](O[C@@H]3O[C@H](CO)[C@H](O)[C@H](O[C@@H]4O[C@H](CO)[C@H](O)[C@H](O)[C@H]4O)[C@H]3NC(C)=O)[C@H](O[C@]3(C(=O)O)C[C@H](O)[C@@H](NC(C)=O)[C@H]([C@H](O)[C@H](O)CO)O3)[C@H]2O)[C@H](O)[C@H]1O)NC(=O)CCCCCCCCCCCCCCCCCCC]

The chemical name for GM2 is (2S,3R,4E)-3-Hydroxy-2-(octadecanoylamino)octadec-4-en-1-yl 2-acetamido-2-deoxy-β-D-galactopyranosyl-(1→4)-[5-acetamido-3,5-dideoxy-D-glycero-α-D-galacto-non-2-ulopyranonosyl-(2→3)]-β-D-galactopyranosyl-(1→4)-β-D-glucopyranoside. GM2 is also known as β-D-GalNAc-(1→4)-[α-Neu5Ac-(2→3)]-β-D-Gal-(1→4)-β-D-Glc-(1↔1)-N-octadecanoylsphingosine. The chemical structure of GM2 is provided as follows:

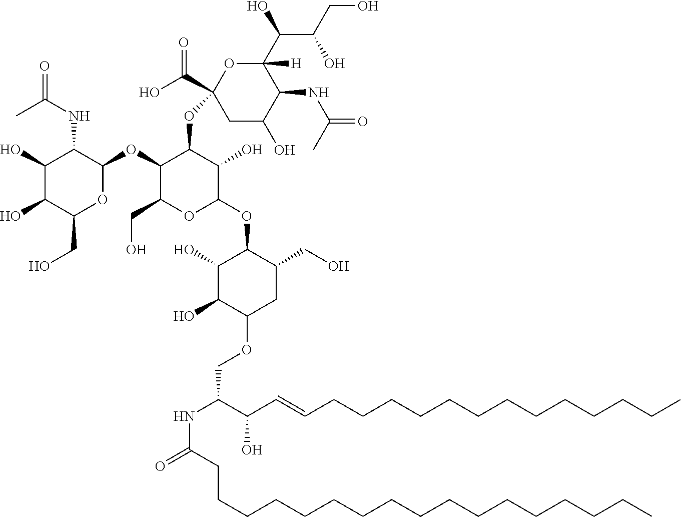 [SMILES: CCCCCCCCCCCCC/C=C/[C@H](O)[C@@H](COC1C[C@@H](CO)[C@H](OC2O[C@@H](CO)[C@@H](O[C@H]3O[C@@H](CO)[C@@H](O)[C@@H](O)[C@@H]3NC(C)=O)[C@@H](O[C@@]3(C(=O)O)CC(O)[C@H](NC(C)=O)[C@@H]([C@@H](O)[C@@H](O)CO)O3)[C@@H]2O)[C@@H](O)[C@@H]1O)NC(=O)CCCCCCCCCCCCCCCCC]

GM3 is known as monosialodihexosylganglioside or as NANA-Gal-Glc-ceramide. The chemical structure for GM3 is provided as follows:

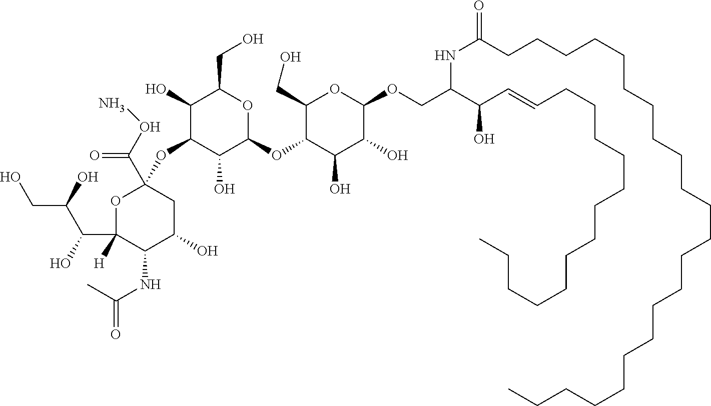 [SMILES: CCCCCCCCCCCCC/C=C/[C@@H](O)C(CO[C@@H]1O[C@H](CO)[C@@H](O[C@@H]2O[C@H](CO)[C@H](O)[C@H](O[C@]3(C(=O)ON)C[C@H](O)[C@H](NC(C)=O)[C@H]([C@H](O)[C@H](O)CO)O3)[C@H]2O)[C@H](O)[C@H]1O)NC(=O)CCCCCCCCCCCCCCCCCCCCCC]

Additional gangliosides that are useful in the membrane compositions are:

GM2-1=aNeu5Ac(2-3)bDGalp(1-4)bDGalNAc(1-4)bDGalNAc(1-4)bDGlcp(1-1)Cer;

GM2,GM2a=bDGalpNAc(1-4)[aNeu5Ac(2-3)]bDGalp(1-4)bDGlcp(1-1)Cer;

GM2b=aNeu5Ac(2-8)aNeu5Ac(2-3)bDGalp(1-4)bDGlcp(1-1)Cer;

GM1,GM1a=bDGalp(1-3)bDGalNAc[aNeu5Ac(2-3)]bDGalp(1-4)bDGlcp(1-1)Cer;

asialo-GM1,GA1=bDGalp(1-3)bDGalpNAc(1-4)bDGalp(1-4)bDGlcp(1-1)Cer;

asialo-GM2,GA2=bDGalpNAc(1-4)bDGalp(1-4)bDGlcp(1-1)Cer;

GM1b=aNeu5Ac(2-3)bDGalp(1-3)bDGalNAc(1-4)bDGalp(1-4)bDGlcp(1-1)Cer;

GD3=aNeu5Ac(2-8)aNeu5Ac(2-3)bDGalp(1-4)bDGlcp(1-1)Cer;

GD2=bDGalpNAc(1-4)[aNeu5Ac(2-8)aNeu5Ac(2-3))]bDGalp(1-4)bDGlcp(1-1)Cer;

GD1a=aNeu5Ac(2-3)bDGalp(1-3)bDGalNAc(1-4)[aNeu5Ac(2-3)]bDGalp(1-4)bDGlcp(1-1)Cer;

GD1alpha=aNeu5Ac(2-3)bDGalp(1-3)bDGalNAc(1-4)[aNeu5Ac(2-6)]bDGalp(1-4)bDGlcp(1-1)Cer;

GD1b=bDGalp(1-3)bDGalNAc(1-4)[aNeu5Ac(2-8)aNeu5Ac(2-3)]bDGalp(1-4)bDGlcp(1-1)Cer;

GT1a=aNeu5Ac(2-8)aNeu5Ac(2-3)bDGalp(1-3)bDGalNAc(1-4)[aNeu5Ac(2-3)]bDGalp(1-4)bDGlcp(1-1)Cer;

GT1,GT1b=aNeu5Ac(2-3)bDGalp(1-3)bDGalNAc(1-4)[aNeu5Ac(2-8)aNeu5Ac(2-3))]bDGalp(1-4)bDGlcp(1-1)Cer;

OAc-GT1b=aNeu5Ac(2-3)bDGalp(1-3)bDGalNAc(1-4)aXNeu5Ac9Ac(2-8)aNeu5Ac(2-3)]bDGalp(1-4)bDGlcp(1-1)Cer;

GT1c=bDGalp(1-3)bDGalNAc(1-4)[aNeu5Ac(2-8)aNeu5Ac(2-8)aNeu5Ac(2-3))]bDGalp(1-4)bDGlcp(1-1)Cer;

GT3=aNeu5Ac(2-8)aNeu5Ac(2-8)aNeu5Ac(2-3)bDGal(1-4)bDGlc(1-1)Cer;

GQ1b=aNeu5Ac(2-8)aNeu5Ac(2-3)bDGalp(1-3)bDGalNAc(1-4)[aNeu5Ac(2-8)aNeu5Ac(2-3))]bDGalp(1-4)bDGlcp(1-1)Cer; and

GGal=aNeu5Ac(2-3)bDGalp(1-1)Cer.

In one embodiment, the membrane composition comprises or consists of at least one of GM1, GM2, and GM3. The membrane composition may be enriched in at least one of GM1, GM2, and GM3. In another embodiment, the membrane composition comprises or consists of GM1. The membrane composition may be enriched in GM1. In another embodiment, the membrane composition comprises or consists of GM2. The membrane composition may be enriched in GM2. In another embodiment, the membrane composition comprises or consists of GM3. The membrane composition may be enriched in GM3. The enrichment of a membrane composition in any one or more gangliosides refers to a membrane composition comprising any of the amounts of ganglioside as previously described, for example based on the total weight % of the ganglioside and lipid and combinations thereof, may be at least about 0.01, 0.1, 0.2, 0.3, 0.4, 0.5, 0.6, 0.7, 0.8, 0.9, 1, 2, 3, 4, 5, 6, 7, 8, 9, or 10% of the total weight.

Lipids

The membrane compositions comprise a lipid. The lipids can be hydrophobic or polar such as amphiphilic. It will be appreciated that the amphiphilic compound has one or more polar head groups connected to one or more non-polar tail groups. The lipids can be cationic, anionic or non-ionic. The lipids may also be surfactants. The lipid may be selected from the group consisting of one or more fatty acids, glycerolipids, glycerophospholipids, sphingolipids, saccharolipids, and polyketides, sterols, prenols, and combinations thereof.

Examples of useful cationic lipids are dimethylammonium chlorides, dimethylammonium bromides, dioctadecyldimethylammonium chloride. Examples of cationic lipids may be N-(2,3-dioleyloxy) propyl-N,N,N-trimethylammonium, didodecylammonium bromide, 1,2-dioleoyloxy-3-trimethylammonio propane, 3β-N—(N′,N′-dimethyl-aminoethane)-carbamol cholesterol, 1,2-dimyristoyloxypropyl-3-dimethylhydroxyethyl ammonium, and 2,3-dioleyloxy-N-[2-(spermine carboxamido) ethyl]-N,N-dimethyl-1-propanaminium trifluoroacetate, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleyloxy)propyl)-N,N,N-trimethylammonium chloride (DOTAP); 313-(N—(N′,N′-dimethylaminoethane)-carbamoyl)cholesterol (DC-Chol) and N-(1,2-dimyristyloxyprop-3-yl)-N,N-dimethyl-N-hydroxyethyl ammonium bromide (DMRIE). Examples of useful anionic lipids are sodium stearate, sodium palminate, sulfated butyl oleate, sodium oleate, salts of fumaric acid, glycerol, hydroxylated lecithin, sodium lauryl sulphate phosphatidylglycerol, cardiolipin, diacylphosphatidylserine, diacylphosphatidic acid, n-dodecanoyl phosphatidylethanolamines, n-succinyl phosphatidylethanolamines, n-glutaryl phosphatidylethanolamines, lysylphosphatidylglycerols, phosphatidic acid, phosphatidylinositol, phosphatidylglycerol, phosphatidyl ethylene glycol, cholesterol succinate, phosphatidyl inositol, phosphatidyl serine, phosphatidyl glycerol, phosphatidic acid, poly(ethylene glycol)-phosphatidyl ethanolamine, dimyristoylphosphatidyl glycerol, dioleoylphosphatidyl glycerol, dilauryloylphosphatidyl glycerol, dipalmitoylphosphatidyl glycerol, distearyloylphosphatidyl glycerol, dimyristoyl phosphatic acid, dipalmitoyl phosphatic acid, dimyristoyl phosphatidyl serine, dipalmitoyl phosphatidyl serine and brain phosphatidyl serine.

Examples of useful non-ionic lipids are glyceryl monoesters, glyceryl diesters, alkoxylated alcohols, alkoxylated alkyl phenols, alkoxylated acids, alkoxylated amides, alkoxylated sugar derivatives, alkoxylated derivatives of natural oils or waxes, polyoxyethylene polyoxypropylene block copolymers, polyoxyethylene fatty ethers, polyoxyethylene ether fatty acids, polyoxyethylene ether fatty acid esters, steroids; fatty acid esters of alcohols. Examples of suitable glyceryl monoesters include, but are not limited to, glyceryl caprate, glyceryl caprylate, glyceryl cocate, glyceryl erucate, glyceryl hydroxysterate, glyceryl isostearate, glyceryl lanolate, glyceryl laurate, glyceryl linolate, glyceryl myristate, glyceryl oleate, glyceryl PABA, glyceryl palmitate, glyceryl ricinoleate, glyceryl stearate, glyceryl thiglycolate, and mixtures thereof. Examples of suitable glyceryl diesters include, but are not limited to, glyceryl dilaurate, glyceryl dioleate, glyceryl dimyristate, glyceryl disterate, glyceryl sesuioleate, glyceryl stearate lactate, and mixtures thereof. Examples of suitable polyoxyethylene fatty ethers include, but are not limited to, polyoxyethylene cetyl/stearyl ether, polyoxyethylene cholesterol ether, polyoxyethylene laurate or dilaurate, polyoxyethylene stearate or distearate, polyoxyethylene lauryl or stearyl ether, and mixtures thereof. Examples of suitable steroids include, but are not limited to, cholesterol, betasitosterol, bisabolol, and mixtures thereof. Examples of suitable fatty acid esters of alcohols include isopropyl myristate, aliphati-isopropyl n-butyrate, isopropyl n-hexanoate, isopropyl n-decanoate, isopropyl palmitate, octyldodecyl myristate, diacylphosphatidylcholine, diacylphosphatidylethanolamine, ceramide, sphingomyelin, cephalin, cholesterol, cerebrosides and diacylglycerols. Another example is diphosphatidyl glycerol.

In an embodiment, the lipid can comprise or consist of a sphingolipid, sterol lipid, phospholipid, glycerol lipid, or combination thereof.

Examples of sphingolipids include, for example, sphingosines, sphinganines, phytosphingosines, sphingoid base homologs and variants, sphingoid base 1-phosphates, lysosphingomyelins and lysoglycosphingolipids, N-methylated sphingoid bases, sphingoid base analogs, ceramides, dihydroceramides, phytoceramides, acylceramides, ceramide 1-phosphates, phosphosphingolipids, sphingomyelins, ceramide phosphoethanolamines, ceramide phosphoinositols, phosphonosphingolipids, neutral glycosphingolipids, acidic glycosphingolipids, gangliosides, sulfatides, glucuronosphingolipids, phosphoglycosphingolipids, basic glycosphingolipids, amphoteric glycosphingolipids, and arsenosphingolipids and derivatives thereof.

Examples of sterol lipids include, for example, phytosterols, 5α-stanols, cholesterol or a combination thereof. The phytosterols may be sitosterol, campesterol, stigmasterol and avenosterol, or a combination thereof. The 5α-stanols may be cholestanol, 5α-campestanol, 5α-sitostanol or a combination thereof. In one embodiment, the sterol lipid may be cholesterol.

Examples of phospholipids include, for example, phosphatidylcholine, a phosphatidylglycerol, a phosphatidylethanolamine, a phosphatidic acid, a sphingomyelin, or combination thereof. The phospholipid may be a phosphatidylcholine (PC). The PC may be 1,2-didodecanoyl-sn-glycero-3-phosphocholine (DLPC), 1,2-ditetradecanoyl-sn-glycero-3-phosphocholine (DMPC), 1,2-didocosenoyl-sn-glycero-3-phosphocholine (DEPC), 1-hexadecanoyl-2-octadecenoyl-sn-glycero-3-phosphocholine (POPC), 1, 2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1-stearoyl-2-hydroxy-sn-glycero-3-phosphocholine (MSPC), L-α-phosphatidylcholine, hydrogenated (Soy) (HSPC), or combination thereof. The phospholipid may be a phosphatidylglycerol (PG). The PG may be 1,2-ditetradecanoyl-sn-glycero-3-[phospho-rac-(1-glycerol)] (DMPG), 1,2-dihexadecanoyl-sn-glycero-3-[phospho-rac-(1-glycerol)] (DPPG), 1,2-dioctadecanoyl-sn-glycero-3-[phospho-rac-(1-glycerol)] (DSPG), 1,2-dioctadecenoyl-sn-glycero-3-[phospho-rac-(1-glycerol)] (DOPG), 1-hexadecanoyl-2-octadecenoyl-sn-glycero-3-[phospho-rac-(1-glycerol)] (POPG), or any combination thereof. The phospholipid may be a phosphatidylethanolamine (PE). The PE may be 1,2-ditetradecanoyl-sn-glycero-3-phosphoethanolamine (DM PE) dipalmitoylphosphatidylethanolamine (DPPE), 1,2-dioctadecanoyl-sn-glycero-3-phosphoethanolamine (DSPE), 1,2-dioctadecenoyl-sn-glycero-3-phosphoethanolamine (DOPE). The phospholipid may be a phosphatidic acid (PA). The PA may be 1,2-ditetradecanoyl-sn-glycero-3-phosphatidic acid (DMPA), 1,2-dihexadecanoyl-sn-glycero-3-phosphatidic acid (DPPA), 1,2-dioctadecanoyl-sn-glycero-3-phosphatidic acid (DSPA), 1,2-dioctadecenoyl-sn-glycero-3-phosphoserine (DOPS), or any combination thereof. The phospholipid may be phosphatidyl serine (PS), phosphatidyl inositol (PI), or any combination thereof.

Examples of glycerol lipids include, for example, monoradylglycerols, monoacylglycerols, monoalkylglycerols, mono-(1Z-alkenyl)-glycerols, diradylglycerols, diacylglycerols, 1-alkyl,2-acylglycerols, 1-acyl,2-alkylglycerols, dialkylglycerols, 1Z-alkenylacylglycerols, di-glycerol tetraethers, di-glycerol tetraether glycans, triradylglycerols, triacylglycerols, alkyldiacylglycerols, dialkylmonoacylglycerols, 1Z-alkenyldiacylglycerols, estolides, glycosylmonoradylglycerols, glycosylmonoacylglycerols, glycosylmonoalkylglycerols, glycosyldiradylglycerols, glycosyldiacylglycerols, glycosylalkylacylglycerols, and glycosyldialkylglycerols, or any combination thereof.

In another embodiment, the lipid can comprise or consist of a sphingosine, ceramide, sphingomyelin, cholesterol, or combination thereof.

### Optional Additional Components

The membrane composition can also comprise other compounds and/or additives. Any other compounds or additives that may be present in the membrane composition may also form part of a membrane layer comprising a ganglioside and lipid, wherein the layer encapsulates the agent preparation or particle thereof. Other non-lipid compounds may arise from the membrane composition material when sourced or made from animals or natural products. Other components can also be added to the membrane composition, and may be sourced or obtained from animals or natural products, or be semi-synthetic or synthetic. In one embodiment, the membrane compositions contain a low amount of triglycerides.

### Method for Making Membrane Compositions

The present invention also provides a method for making a membrane composition from a membrane composition source. The method can comprise the steps of:

(a) contacting a membrane composition source comprising a lipid and ganglioside with an alcohol solution to provide a liquid mixture comprising insoluble solids;

(b) separating the insoluble solids from the liquid mixture to obtain an alcohol solution comprising a lipid and ganglioside; and

(c) optionally removing at least some of the alcohol from the alcohol solution to obtain the membrane composition.

The membrane composition source can be a natural source comprising gangliosides and lipids, for example a plant or animal source, such as milk. In one embodiment, the membrane composition source is milk or nerve tissue. The extract from milk can be an extract from butter milk powder or other milk product comprising milk fat globule membrane (MFGM). The nerve tissue can be from a mammal or non-mammal such as felines, bovines, pigs, horses and fish. The membrane composition source can be a powder, such as butter milk powder or whole milk powder, which may also be spray dried powder. It will be appreciated that butter milk powder is produced from the liquid recovered during butter production, for example the liquid component recovered when a concentration of fat is removed from milk in the form of butter.

The insoluble solids may comprise at least one of proteins, sugars and salts. The contacting step may comprise one or more steps of contacting the membrane composition source with the alcohol solution to extract at least a portion of the membrane composition into the alcohol solution. The removing step may comprise evaporating the alcohol from the alcohol solution to increase the concentration of the membrane composition (or ganglioside and/or lipid) in the alcohol solution.

The alcohol solution may comprise one or more alcohols (i.e. alcoholic solvents). The alcohol solution may be an aqueous alcohol solution, for example comprising water and one or more alcohols (i.e. one or more alcoholic solvents). The alcohol (which may also referred to as an alcoholic solvent) can be a straight or branched hydrocarbon with one or more hydroxyl groups. The alcohol can be a straight chained or branched C₁₋₇ alcohol (e.g. C₁₋₇alkanol), for example amyl alcohol, n-pentanol or isopropanol. In one embodiment, the alcohol is isopropanol. Prior to contacting with the membrane composition source, the alcohol solution may comprise or consist of isopropanol or may comprise or consist of ethyl acetate. The aqueous alcohol solution may contain water in less than (by weight % of the total solution) 90, 80, 70, 50, 40, 30, 20, 15, 10, 9, 8, 7, 6, 5, 4, 3, 2, 1, 0.5, 0.1, 0.05, 0.01, 0.005, or 0.001.

### Agent Preparation

The agent preparation comprises an agent and optionally one or more excipients. The agent preparation can exist as, be provided as or be formed into particles. Each particle comprises an agent and can optionally further comprise an excipient. A plurality of encapsulated particles can be provided wherein each particle can be formed from a preparation (e.g. composition) comprising an agent and optionally an excipient. For example, a single particle may have a composition comprising both the agent and excipient, and be individually encapsulated within a membrane composition, as previously described above in relation to FIGS. 4-6. In other words, each particle can be individually encapsulated within a layer comprising a ganglioside and lipid. In another embodiment, a plurality of particles may be encapsulated together within a membrane composition. The agent preparation may be provided in the form of a liquid, gel or solid. In one embodiment, the agent preparation is provided in the form of a gel or solid. It will be appreciated that the membrane composition can provide a coating or encapsulate the gel or solid.

The one or more optional excipients of the agent preparation can be selected from a gelling agent, buffering agent, or other excipient compound or agent. In one embodiment, the one or more optional excipients comprise a gelling agent.

The gelling agent can be a compound or composition that is non-harmful, biologically acceptable, or biologically beneficial to the cellular environment. The gelling agent can be derivatized guar gum (e.g., carboxymethyl guar, carboxymethylhydroxyethyl guar, and carboxymethylhydroxypropyl guar); a cellulose derivative (e.g., hydroxyethyl cellulose, carboxyethylcellulose, carboxymethylcellulose, and carboxymethylhydroxyethylcellulose); xanthan; succinoglycan; alginate; chitosan; starch; agar; gelatin; any derivative thereof; and any combination thereof. For example, the gelling agent can be a carbohydrate based polymer such as a glucose polymer, for example glycogen. The gelling agent can be a starch, for example a pre-gelatinised starch.

In an embodiment, the gelling agent is a pH sensitive gelling agent. The pH sensitive gelling agent can comprise or consist of pectin or modified pectin. It will be appreciated that the pectin or modified pectin can be naturally sourced from fruit and vegetables, which may be in the form a water soluble high molecular weight colloidal carbohydrates or polysaccharides. The pectin or modified pectin can be an acidic hemicellulose. The pectin or modified pectin can be a gelling agent used in food preparation or as a food ingredient (GRAS). The pectin may be obtained, for example, from apples or citrus peel, or from plant cells where pectins are widely distributed. Pectins may have a high proportion of D-galacturonic acid residues which may confer further advantages such as an enhanced pH sensitivity of gel strength. This may be modified by forming methyl esters or amides (with ammonia). All these forms of pectin may be used, although additional advantages may be provided by amide esters.

In another embodiment, the gelling agent is a heat sensitive gelling agent. It will be appreciated that the gel forming temperature varies with different heat sensitive gelling agents. For example, the heat sensitive gelling agent can comprise or consist of gelatin or modified gelatin. It will be appreciated that the gelatin or modified gelatin can be naturally derived from animal skin, bones and tendons.

The heat sensitive gelling agent may also be derivatized from polymers (e.g.polyethylene glycol-polylactic acid, glycolic acid-polyethylene glycerol, poly(N-isopropylacrylamide), poloxamers (e.g.triblock poly(ethylene oxide)-poly(propylene oxide)-poly(ethylene oxide), gums (e.g. xanthan gum), and hemicelluloses (e.g. xyloglucan). The heat sensitive gelling agents may be modified by changing the ratio of hydrophilic and hydrophobic segments, block length and polydispersity of the polymers.

In yet another embodiment, the properties of the gelling agent can be altered by various physical factors such as temperature and pH resulting in the controlled delivery of the agent. For example, the gelling agent may be configured to break down and decrease in gelling efficacy after a predetermined time, resulting in the breaking up of the particle and release of the agent. In another example, a heat-sensitive gelling agent may comprise a thermoresponsive polymer (e.g. poly(N-isopropylacrylamide), which exhibits a change in physical properties with temperature, resulting in the shrinkage of the particle and subsequent release of the agent, at a predetermined temperature. It will be appreciated that the gelling agent can be modified to alter its gelling properties the release rate of the agent from the particle.

Other suitable gelling agents, including other suitable heat sensitive gelling agents, can be found generally in Pharmaceutical Manufacturing Handbook: Production and Processes, Gad, S. C., ed. John Wiley & Sons, Inc., Hoboken, N.J., 2008. Hydrogen ion transport may occur across an encapsulating layer provided by the membrane composition. An excipient may be provided in the agent preparation and be pH sensitive to facilitate or activate release of one or more agents encapsulated by the membrane composition. For example, in an environment or in some embodiments having a pre-determined pH, in which encapsulated agent preparation or an encapsulated particle thereof may be present, the transport of hydrogen ions may occur across the encapsulating layer (e.g. membrane comprising ganglioside and lipid) and then alter the properties of agent preparation or excipient encapsulated therein (e.g. converting a gel excipient to a liquid) to initiate breakdown of the encapsulated agent preparation (or encapsulated particle thereof) and release the agent from its protective encapsulating layer into the external environment. For example, in a particle containing both an agent and an excipient, the excipient in that particle can provide or facilitate release of the agent contained in that same particle by disrupting the encapsulating membrane layer of the membrane composition. The excipient can therefore be used for predetermined or targeted delivery and release into the lymphatic system, cardiovascular system, or transport through the lymphatic system of intact encapsulated particles for delivery to intended cells, tissues or organs. For example, a pH sensitive gelling agent according to at least some embodiments as described herein may facilitate or provide such further properties or advantages.

Agents

The agent in the agent preparation can be a pharmaceutically acceptable agent. The pharmaceutically acceptable agent may be any biologically active substance that is useful for diagnosis, mitigation, treatment, or prevention of disease, or that can affect the structure or any function of the body. The agent may be a therapeutic agent including small and large molecule agents, diagnostic agent, nutritional agent, cosmetic agent, food or food ingredient, dye, or combination thereof. The agent may be a hydrophobic agent, hydrophilic agent, or amphipathic agent.

The agent preparation may comprise or consist of one or more agents. In one embodiment, the agent is a therapeutic agent, diagnostic agent, nutritional agent, cosmetic agent, food or food ingredient, or combination thereof. Examples of agents include biological products such as antibodies, monoclonal antibodies, antibody fragments, antibody drug conjugates, proteins, biologically active proteins, fusion proteins, recombinant proteins, peptides, polypeptides, synthesized polypeptides, vaccines, blood products, blood components or derivatives, therapeutic serums, viruses, toxins, antitoxins, polynucleotides, cells or part or parts thereof, stem cells as well as small molecules.

The polynucleotide may be a sense polynucleotide, an antisense polynucleotide, a double stranded DNA (dsDNA) or a double stranded RNA (dsRNA). In one example, the dsDNA or dsRNA is an aptamer. In one example, the dsRNA is a siRNA, miRNA or shRNA.

In one example, the agent is a polypeptide which is a binding protein. In one example, the binding protein is an antibody or antigenic binding fragment. The antibody may be a monoclonal antibody, humanized antibody, chimeric antibody, single chain antibody, diabody, triabody, or tetrabody. In an embodiment, the antibody may be a bi-specific antibody, an engineered antibody, an antibody-drug conjugate or a biosimilar antibody. In one embodiment, the agent may be a monoclonal antibody. In an embodiment, the antibody may be Abatacept, Abciximab, Alirocumab, Adalimumab, Afibercept, Alemtuzumab, Basiliximab, Belimumab, Bevacizumab (Avastin), Brentuximab vedotin, Bococizumab, Canakinumab, Cetuximab, Certolizumab pegol, Daclizumab, Daratumumab, Denosumab, Durvalumab, Eculizumab, Efalizumab, Elotuzumab, Etanercept, Evolocumab, Golimumab, Ibritumomab tiuxetan, Infliximab, Ipilimumab, Muromonab-CD3, Natalizumab, Nivolumab, Ocrelizumab, Ofatumumab, Omalizumab, Pembrolizumab, Palivizumab, Panitumumab, Pidilizumab, Ranibizumab, Rituximab, Tocilizumab (or Atlizumab), Tositumomab, Trastuzumab, Tremelimumab Ustekinumab, Vedolizumab, or a biosimilar thereof.

The polypeptide may be a cytokine or chemokine. The cytokine or chemokine may be bone morphogenetic protein, erythropoietin, granulocyte colony-stimulating factor, granulocyte macrophage colony-stimulating factor, thrombopoietin, IFNα, IFNβ, IFNλ, IFNγ, TNFα, TNFβ, thymic stromal lymphopoietin or one or more interleukins.

The polypeptide may be a hormone. The hormone may be epinephrine, melatonin, triiodothyronine, thyroxine, prostaglandin, leukotrienes, prostacyclin, thromboxane, amylin, anti-müllerian hormone, adponectin, adrenocorticotropic hormone, angiotensinogen, angiotensin, atrial-natriuretic peptide, brain natriuretic pepeptide, calcitonin, cholecystokinin, corticotropin-releasing hormone, cortistatin, encephalin, endothelin, erythropoietin, folcle-stimulating hormone, galanin, glucagon, glucagon-like peptide-1, gonadotropin-releasing hormone, growth hormone-releasing hormone, hepcidin, human chorionc gonadotropin, human placental lactogen, growth hormone, inhibin, insulin, insulin-like groth factor, leptin, luteinizing hormone, melanocyte stimulating hormone, motilin orexin, oxytocin, pancreatic polypeptide, pituitary adenylate cyclase-activating peptide, prolactin, prolactin releasing hormone, relaxin, renin, secretin, somatostatin, thrombopoietin, thyroid-stimulating hormone, thyrotropin-releasing hormone, vasoactive intestinal peptide or a derivative or analogue thereof.

The polypeptide may be a blood coagulation factor. The coagulation factor may be factor I, factor II, factor III, factor IV, factor V, factor VI, factor VII, factor VIII, factor IX, factor X, factor XII, factor XIII, high-molecular-weight kininogen, fibronectin, antithrombin II, heparin cofactor II, protein C, protein S, protein Z, protein Z-related protease inhibitor, plasminogen, alpha 2-antiplasmin, tissue plasminogen activator, urokinase, plasminogen activator inhibitor-1 or plasminogen activator inhibitor 2.

The polypeptide may be an enzyme. The enzyme may be a protease, lipase, asparaginase, liprotamase, tissue plasminogen activator, collagenase, glutaminase, hyaluronidase, streptokinase, uricase, urokinase or nuclease, such as a programmable nuclease. The enzyme may be a programmable nuclease targeted to introduce a genetic modification into a gene or a regulator region thereof. The programmable nuclease may be a RNA-guided engineered nuclease (RGEN). The RGEN may be from an archaeal genome or may be a recombinant version thereof. The RGEN may be from a bacterial genome or is a recombinant version thereof. The RGEN may be from a Type I (CRISPR)-cas (CRISPR-associated) system. The RGEN may be from a Type II (CRISPR)-cas (CRISPR-associated) system.

The RGEN may be from a Type III (CRISPR)-cas (CRISPR-associated) system. The nuclease may be from a class I RGEN. The nuclease may be from a class II RGEN. The programmable nuclease may be targeted by one or more RNA, DNA or RNA/DNA molecules which may comprise one or more base analogues or modified bases.

The agent may be an antigen which stimulates an immune response in a subject. The antigen may be a protein, peptide, polysaccharide or oligosaccharide (free or conjugated to a protein carrier), or mixtures thereof. The antigen may also be a cell or part thereof or a viral particle or part thereof. The antigen may be a plant antigen (such as a pollen), a viral antigen, a bacterial antigen, a fungal antigen, a protozon antigen, or a tumor antigen. The antigen may act as a vaccine, stimulating the production of antibodies providing immunity against one or more diseases or infections.

Bacterial antigens can be derived from bacteria, including but not limited to, Helicobacter pylori, Chlamydia pneumoniae, Chlamydia trachomatis, Ureaplasma urealyticum, Mycoplasma pneumoniae, Staphylococcus spp., Staphylococcus aureus, Streptococcus spp., Streptococcus pyogenes, Streptococcus pneumoniae, Streptococcus viridans, Enterococcus faecalis, Neisseria meningitidis, Neisseria gonorrhoeae, Bacillus anthracis, Salmonella spp., Salmonella typhi, Vibrio cholera, Pasteurella pestis, Pseudomonas aeruginosa, Campylobacter spp., Campylobacter jejuni, Clostridium spp., Clostridium difficile, Mycobacterium spp., Mycobacterium tuberculosis, Treponema spp., Borrelia spp., Borrelia burgdorferi, Leptospira spp., Hemophilus ducreyi, Corynebacterium diphtheria, Bordetella pertussis, Bordetella parapertussis, Bordetella bronchiseptica, hemophilus influenza, Escherichia coli, Shigella spp., Erlichia spp., and Rickettsia spp. The bacterial antigen can be native, recombinant or synthetic. Such bacterial antigens include, but are not limited to, selectins or lectins from bacteria that bind to carbohydrate determinants present on cell surfaces, and bacteria receptors for proteins, such as fibronectin, laminin, and collagens.

Viral antigens can be derived from viruses, including but not limited to, Influenza viruses, a Parainfluenza viruses, Mumps virus, Adenoviruses, Respiratory syncytial virus, Epstein-Barr virus, Rhinoviruses, Polioviruses, Coxsackieviruses, Echoviruses, Rubeola virus, Rubella virus, Varicell-zoster virus, Herpes viruses (human and animal), Herpes simplex virus, Parvoviruses (human and animal), Cytomegalovirus, Hepatitis viruses, Human papillomavirus, Alphaviruses, Flaviviruses, Bunyaviruses, Rabies virus, Arenaviruses, Filoviruses, HIV 1, HIV 2, HTLV-1, HTLV-II, FeLV, Bovine LV, FeIV, Canine distemper virus, Canine contagious hepatitis virus, Feline calicivirus, Feline rhinotracheitis virus, TGE virus (swine), and Foot and mouth disease and other viruses as herein described. Viral antigens can be native, recombinant or synthetic. Such viral antigens include, but are not limited to, a virus particle or part thereof, viral DNA or RNA, or viral proteins that are responsible for attachment to cell surface receptors to initiate the infection process, such as (i) envelope glycoproteins of retroviruses (HIV, HTLV, FeLV and others) and herpes viruses, and (ii) the neuramidase of influenza viruses.

Tumor associated antigens can be native, recombinant or synthetic. Such tumor associated antigens include, but are not limited to, MUC-1 and peptide fragments thereof, protein MZ2-E, polymorphic epithelial mucin, folate-binding protein LK26, MAGE-1 or MAGE-3 and peptide fragments thereof, Human chorionic gonadotropin (HCG) and peptide fragments thereof, Carcinoembryonic antigen (CEA) and peptide fragments thereof, Alpha fetoprotein (AFP) and peptide fragments thereof, Pancreatic oncofetal antigen and peptide fragments thereof, CA 125, 15-3,19-9, 549, 195 and peptide fragments thereof, Prostate-specific antigens (PSA) and peptide fragments thereof, Prostate-specific membrane antigen (PSMA) and peptide fragments thereof, Squamous cell carcinoma antigen (SCCA) and peptide fragments thereof, Ovarian cancer antigen (OCA) and peptide fragments thereof, Pancreas cancer associated antigen (PaA) and peptide fragments thereof, Her1/neu and peptide fragments thereof, gp-100 and peptide fragments thereof, mutant K-ras proteins and peptide fragments thereof, mutant p53 and peptide fragments thereof, nonmutant p53 and peptide fragments thereof, truncated epidermal growth factor receptor (EGFR), chimeric protein p210BCR-ABL, telomerase and peptide fragments thereof, suvivin and peptide fragments thereof, Melan-A/MART-1 protein and peptide fragments thereof, WT1 protein and peptide fragments, LMP2 protein and peptide fragments, HPV E6 E7 protein and peptide fragments, HER-2/neu protein and peptide fragments, Idiotype protein and peptide fragments, NY-ESO-1 protein and peptide fragments, PAP protein and peptide fragments, cancer testis proteins and peptide fragments, and 5T4 protein and peptide fragments. Tumor antigens can be native, recombinant or synthetic.

Protozoan antigens can be derived from protozoans, such as, but not limited to Babeosis bovis, Plasmodium, Leishmania spp. Toxoplasma gondii, and Trypanosoma cruzi and can be native, recombinant or synthetic.

Fungal antigens can be derived from fungi, such as, but not limited to, Aspergillus sp., Candida albicans, Cryptococcus neoformans, and Histoplasma capsulatum and can be native, recombinant or synthetic.

The agent may be a cell or a part thereof. The cell may be a eukaryotic cell. The cell may be a prokaryotic cell. The prokaryotic cell may be a bacterial cell. The cell may stimulate an immune response in the subject.

The agent may an antineoplastic agent. A partial listing of some useful and commonly known commercially approved (or in active development) antineoplastic agents by classification is as follows.

Structure-Based Classes: Fluoropyrimidines-5-FU, Fluorodeoxyuridine, Ftorafur, 5′-deoxyfluorouridine, UFT, S-1 Capecitabine; pyrimidine Nucleosides-Deoxycytidine, Cytosine Arabinoside, 5-Azacytosine, Gemcitabine, 5-Azacytosine-Arabinoside; Purines-6-Mercaptopurine, Thioguanine, Azathioprine, Allopurinol, Cladribine, Fludarabine, Pentostatin, 2-Chloro Adenosine; Platinum Analogues-Cisplatin, Carboplatin, Oxaliplatin, Tetraplatin, Platinum-DACH, Ormaplatin, CI-973, JM-216; Anthracyclines/Anthracenediones-Doxorubicin, Daunorubicin, Epirubicin, Idarubicin, Mitoxantrone; Epipodophyllotoxins—Etoposide, Teniposide; Camptothecins—Irinotecan, Topotecan, Lurtotecan, Silatecan, 9-Amino Camptothecin, 10,11-Methylenedioxy Camptothecin, 9-Nitro Camptothecin, TAS 103, 7-(4-methyl-piperazino-methylene)-10,11-ethylenedioxy-20(S)-camptothecin, 7-(2-N-isopropylamino)ethyl)-20(S)-camptothecin; Hormones and Hormonal Analogues-Diethylstilbestrol, Tamoxifen, Toremefine, Tolmudex, Thymitaq, Flutamide, Bicalutamide, Finasteride, Estradiol, Trioxifene, Droloxifene, Medroxyprogesterone Acetate, Megesterol Acetate, Aminoglutethimide, Testolactone and others; Enzymes, Proteins and Antibodies—Asparaginase, Interleukins, Interferons, Leuprolide, Pegaspargase, and others; Vinca Alkaloids—Vincristine, Vinblastine, Vinorelbine, Vindesine; Taxanes—Paclitaxel, Docetaxel.

Mechanism-Based Classes: Antihormonals—See classification for Hormones and Hormonal Analogues, Anastrozole; Antifolates—Methotrexate, Aminopterin, Trimetrexate, Trimethoprim, Pyritrexim, Pyrimethamine, Edatrexate, MDAM; Antimicrotubule Agents—Taxanes and Vinca Alkaloids; Alkylating Agents (Classical and Non-Classical)—Nitrogen Mustards (Mechlorethamine, Chlorambucil, Melphalan, Uracil Mustard), Oxazaphosphorines (Ifosfamide, Cyclophosphamide, Perfosfamide, Trophosphamide), Alkylsulfonates (Busulfan), Nitrosoureas (Carmustine, Lomustine, Streptozocin), Thiotepa, Dacarbazine and others; Antimetabolites—Purines, pyrimidines and nucleosides, listed above; Antibiotics—Anthracyclines/Anthracenediones, Bleomycin, Dactinomycin, Mitomycin, Plicamycin, Pentostatin, Streptozocin; topoisomerase Inhibitors—Camptothecins (Topo I), Epipodophyllotoxins, m-AMSA, Ellipticines (Topo II); Antivirals—AZT, Zalcitabine, Gemcitabine, Didanosine, and others; Miscellaneous Cytotoxic Agents—Hydroxyurea, Mitotane, Fusion Toxins, PZA, Bryostatin, Retinoids, Butyric Acid and derivatives, Pentosan, Fumagillin, and others. The small molecule may be an anthracycline drug, doxorubicin, daunorubicin, mitomycin C, epirubicin, pirarubicin, rubidomycin, carcinomycin, N-acetyladriamycin, rubidazone, 5-imidodaunomycin, N-acetyldaunomycine, daunoryline, mitoxanthrone; a camptothecin compound, camptothecin, 9-aminocamptothecin, 7-ethylcamptothecin, 10-hydroxycamptothecin, 9-nitrocamptothecin, 10,11-methyl enedioxyc amptothecin, 9-amino-10,11-methylenedioxycamptothecin, 9-chloro-10,11-methylenedioxycamptothecin, irinotecan, topotecan, lurtotecan, silatecan, (7-(4-methylpiperazinomethylene)-10,11-ethylenedioxy-20(S)-camptothecin, 7-(4-methylpiperazinomethylene)-10,11-methylenedioxy-20(S)-camptothecin, 7-(2-N-isopropylamino)ethyl)-(20S)-camptothecin; an ellipticine compound, ellipticine, 6-3-aminopropyl-ellipticine, 2-diethylaminoethyl-ellipticinium and salts thereof, datelliptium, retelliptine.

The agent may be a therapeutic or pharmaceutical agent including, without limitation any of the following: antihistamine ethylenediamine derivatives (bromphenifamine, diphenhydramine); Anti-protozoal: quinolones (iodoquinol); amidines (pentamidine); antihelmintics (pyrantel); anti-schistosomal drugs (oxaminiquine); antifungal triazole derivatives (fliconazole, itraconazole, ketoconazole, miconazole); antimicrobial cephalosporins (cefazolin, cefonicid, cefotaxime, ceftazimide, cefuoxime); antimicrobial beta-lactam derivatives (aztreopam, cefmetazole, cefoxitin); antimicrobials of erythromycine group (erythromyc in, azithromycin, clarithromycin, oleandomycin); penicillins (benzylpenicillin, phenoxymethylpenicillin, cloxacillin, methicillin, nafcillin, oxacillin, carbenicillin); tetracyclines; other antimicrobial antibiotics, novobiocin, spectinomycin, vancomycin; antimycobacterial drugs: aminosalicycic acid, capreomycin, ethambutol, isoniazid, pyrazinamide, rifabutin, rifampin, clofazime; antiviral adamantanes: amantadine, rimantadine; quinidine derivatives: chloroquine, hydroxychloroquine, promaquine, qionone; antimicrobial qionolones: ciprofloxacin, enoxacin, lomefloxacin, nalidixic acid, norfloxacin, ofloxacin; sulfonamides; urinary tract antimicrobials: methenamine, nitrofurantoin, trimetoprim; nitroimidazoles: metronidazole; cholinergic quaternary ammonium compounds (ambethinium, neostigmine, physostigmine); anti-Alzheimer aminoacridines (tacrine); anti-Parkinsonal drugs (benztropine, biperiden, procyclidine, trihexylhenidyl); anti-muscarinic agents (atropine, hyoscyamine, scopolamine, propantheline); adrenergic dopamines (albuterol, dobutamine, ephedrine, epinephrine, norepinephrine, isoproterenol, metaproperenol, salmetrol, terbutaline); ergotamine derivatives; myorelaxants or curane series; central action myorelaxants; baclophen, cyclobenzepine, dentrolene; nicotine; beta-adrenoblockers (acebutil, amiodarone); benzodiazepines (ditiazem); antiarrhythmic drugs (diisopyramide, encaidine, local anesthetic series-procaine, procainamide, lidocaine, flecaimide), quinidine; ACE inhibitors: captopril, enelaprilat, fosinoprol, quinapril, ramipril; antilipidemics: fluvastatin, gemfibrosil, HMG-coA inhibitors (pravastatin); hypotensive drugs: clonidine, guanabenz, prazocin, guanethidine, granadril, hydralazine; and non-coronary vasodilators: dipyridamole.

In addition to the above, the anticancer agent may include without any limitation, any topoisomerase inhibitor, vinca alkaloid, e.g., vincristine, vinblastine, vinorelbine, vinflunine, and vinpocetine, microtubule depolymerizing or destabilizing agent, microtubule stabilizing agent, e.g., taxane, aminoalkyl or aminoacyl analog of paclitaxel or docetaxel, e.g., 2′-[3-(N,N-diethylamino)propionyl]paclitaxel, 7-(N,N-dimethylglycyl)paclitaxel, and 7-L-alanylpaclitaxel, alkylating agent, receptor-binding agent, tyrosine kinase inhibitor, phosphatase inhibitor, cycline dependent kinase inhibitor, enzyme inhibitor, aurora kinase inhibitor, nucleotide, polynicleotide, and famesyltransferase inhibitor.

The therapeutic agent may be an anthracycline compound or derivatives thereof, camptothecine compounds or derivatives, ellipticine compounds or derivatives, vinca alkaloinds or derivatives, wortmannin, its analogs and derivatives, or pyrazolopyrimidine compounds with the aurora kinase inhibiting properties.

The agent may also be a pre-agent, e.g., a pro-drug or an agent that is capable of being converted to a desired entity upon one or more conversion steps under a condition such as a change in pH or an enzymatic cleavage of a labile bond. Such conversion may occur after the release of the pro-drug from the membrane composition at an intended site of the drug action.

The agent may be a neurological agent useful for treating one or more neurological disorders, such as Alzheimer's disease, Parkinson's disease, Epilepsy or a lysosomal storage disorder e.g. Hurler syndrome. For example, the small molecule or therapeutic agent may be one of dopamine for use in treating Parkinson's disease, or may be one of muscimol, AP5 and GABA, which may be used in treating epilepsy. It will be appreciated that the agents may include pharmaceutically acceptable salts thereof.

In some embodiments the agents are effective for treating one or more of the following neurologic disorders: hereditary and congenital pathologies, demyelinating and degenerative disorders, infections, and neoplasms; headache disorders such as migraine, cluster headache and tension headache; epilepsy and seizure disorders; neurodegenerative disorders, including Alzheimer's disease, Parkinson's disease, Lou Gehrig's disease and ataxia; cerebrovascular diseases such as transient ischemic attacks (TIA) and cerebrovascular accidents (CVA) also known as strokes or brain attack which is either ischemic or hemorrhagic in nature; sleep disorders (insomnia); cerebral palsy (CP), a non-progressive disorder of voluntary and posture control; CNS infections such as encephalitis, meningitis and peripheral neuritis; brain abscess; herpetic meningoencephalitis, aspergilloma and cerebral hydatic cyst; PNS infections, such as tetanus and botulism; neoplasms such as glioblastoma multiforme which is the most malignant brain tumor, spinal cord tumors, peripheral nerves tumor (acoustic neuroma); movement disorders such as Parkinson's disease, chorea, hemiballismus, tic disorder, and Gilles de la Tourette syndrome; CNS demyelinating disease such as multiple sclerosis, and of the peripheral nervous system, such as Guillain-Barr syndrome (GBS) and chronic inflammatory demyelinating polyneuropathy (CIDP); spinal cord disorders e.g. tumors, infections, trauma, malformations such as myelocele, meningomyelocele, myelomeningocele; peripheral nerve disorders like Bell palsy (CN VII) and carpal tunnel syndrome (CTS) involving the median nerve, myopathy and neuromuscular junctions problem (e.g. myasthenia gravis); traumatic injuries to the brain, spinal cord and peripheral nerves; altered mental status, encephalopathy, stupor and coma; and any speech and language disorders (expressive or receptive aphasia).

Agents may be one or more of diazepam, clonazepam, methamphetamine, dextroamphetamine, amphetamine, gabapentin, methylphenidate, phenytoin, lamotrigine, aripiprazole, divalproex, phenothiazines, risperidone, benzodiazepines, topiramate, triamcinolone, levodopa, dopamine agonists, dopa decarboxylase inhibitor, MAO-B inhibitors, COMT inhibitor, carbidopa, benserazide, tolcapone. Dopamine agonists include apomorphine, bromocriptine, pergolide, pramipexole, ropinirole, piribedil, cabergoline, apomorphine and lisuride. MAO-B inhibitors include selegiline and rasagiline, amantadine and anticholinergics such as clozapine, cholinesterase inhibitors, and modafinil.

Agents may be selected that are effective for treating a disorder of the adipose tissue. Disorders of the adipose tissue include lipoma, metabolic syndrome, obesity and diabetes. Agents suitable for treating adipose tissue may be one or more of the following agents: thiazolidinediones, angiogenesis inhibitors such as VEGF or ALS-L1023, growth hormone analogues such as AOD9604, Beta-3 adrenergic receptor agonists such as L-796568, leptin or adiponectin.

Agents may be selected that are effective for treating a lysosomal storage disorder. Agents may be a treatment for a lysosomal storage disorder that is unable to cross the blood brain barrier or unable to cross the blood brain barrier in sufficient quantities for effective treatments. Agents suitable for treating a lysosomal storage disorder include Mucopolysaccharidosis type VII.

Small Molecule Agents

In some embodiments the agent is a small molecule. A “small molecule” agent typically has a molecular weight of less than about 800 Da. The small molecule agent may have, for example, a molecular weight of less than about 700, 600, 500, 400, or 300 Da. The small molecule agent may have, for example, a molecular weight of greater than about 100, 200, 300, 400, or 500 Da. The small molecule agent may have, for example, a molecular weight range (in Da) of from about 50 to 800, 100 to 700, 150 to 600, or 200 to 500 Da.

Large Molecule Agents

In some embodiments the agent is a large molecule. A “large molecule” agent typically has a molecular weight of greater than about 900 Da. The large molecule agent may have, for example, a molecular weight of greater than about 1, 2, 10, 20, 50, 100, 125 or 150 kDa. The large molecule agent may have, for example, a molecular weight of less than about 500, 200, 100, 50, 20, 15, 10, or 5 kDa. The large molecule agent may have, for example, a molecular weight range (in kDa) of from about 1 to 500, 2 to 200, 5 to 100, or 10 to 50 kDa.

In one embodiment, the agent is a therapeutic agent having a molecular weight ranging from about 1 kDa to about 300 kDa. The therapeutic agent may have, for example, a molecular weight of greater than about 1, 2, 10, 20, 50, or 100 kDa. The therapeutic agent may have, for example, a molecular weight of less than about 200, 100, 50, 20, 15, 10, or 5 kDa. The therapeutic agent may have, for example, a molecular weight range (in kDa) of from about 1 to 300, 2 to 200, 3 to 100, 4 to 50, or 5 to 20.

Hydrophilic Agents

The hydrophilic agent may be a small molecule or a large molecule. Hydrophilic agents (also referred to as a lipophobic agent) may include, but are not limited to, agents such as doxorubicin, patenol (olopatadine), macugen (pegaptanib), alphagan (brimonidine tartrate), xeloda (capecitabine), remicade (infliximab), humira (adalimumab), lyrica (pregabalin), insulin and cubicin (daptomycin). The hydrophilic agent may have an HLB above about 10, 11, 12, 13, 14, or 15, or as otherwise described herein. In various embodiments, the hydrophilic agent may have an HLB between about 10 and 20, between about 11 and 20, between about 12 and 20, between about 13 and 20, or between about 14 and 20.

Hydrophobic Agents

The hydrophobic agent may be a small molecule or a large molecule. Hydrophobic agents (also referred to as a lipophilic agent) may include, but are not limited to, agents having a hydrophobic nature and very low or limited water solubility, including those for example used in oncological treatment (e.g. paclitaxel, docetaxel and doxorubicine), anti-mycosis (e.g. anfotericina B), hormones (e.g. progesterone) and the anaesthetics (e.g. propofol). Other examples include prostaglandines, isosorbide dinitrate, testosterone, nitroglycerine, estradiol, vitamin E, cortisone, dexametasone and its esters, and betametasone valerate. The hydrophobic agent may also include therapeutic agents such as ubidecarenone (coenzyme Q10), taxanes such as paclitaxel, dificid (fidaxomicin), patenol (olopatadine), lumigan (bimastoprost), travatan Z (travoprost), zyclara (imiguimod), restasis (cyclosporine). The hydrophobic agent may have an HLB below about 9, 8, 7, 6, 5, 4, or 3, or as otherwise described herein. In various embodiments, the hydrophobic agent may have an HLB between about 0 and 10, between about 0 and 8, between about 0 and 6, or between about 0 and 4.

Diagnostic Agents

In some embodiments the agent is a diagnostic agent. A diagnostic agent is any agent that can be used in the process of determining which disease or condition explains a subject's symptoms. The diagnostic agent may be a hydrophobic, hydrophilic or amphipathic diagnostic agent. The diagnostic agent may be a targeted agent, one directed to a specific location within a subject when administered or a non-targeted diagnostic agent, one which is widely distributed in a subject when administered. The diagnostic agent may be a peptide, polypeptide or small molecule. In an embodiment, the polypeptide is a binding protein. The binding protein may be an antibody or antigenic binding fragment. The antibody may be a monoclonal antibody, humanized antibody, single chain antibody, diabody, triabody, or tetrabody. The diagnostic agent may be a small molecule.

Non-targeted diagnostic agents include, but are not limited to, contrast agents, non-specific gadolinium chelates, PET agents e.g. H₂O-(¹⁵O). In an embodiment, the diagnostic agent is suitable for bioimaging.

The diagnostic agent may be suitable for use with imaging techniques such as scintigraphy, positron emission tomography (PET), computerized tomography (CT) and magnetic resonance imaging (MRI).

The diagnostic agent may be adreView, AMYViD, definity, dotarem, florbetaben F 18, florbetapir F18, fluemetamol F18, gadoterate meglumine, iobenguane I 123, isosulfan blue, lexiscan, lymphazurin, lymphoseek, neuraceq, optison, perflutren, regadenoson, technetium Tc99m tilmanocept or vizamyl.

The diagnostic agent may recognize a molecule in the subject and form a complex which is detectable in body fluid sample form the subject, such as whole blood, plasma, serum or urine. The presence of the complex in the subject's body fluid sample may indicate a subject is positive or negative for a disease or condition.

Nutritional Agents

In some embodiments the agent is a nutritional agent. The nutritional agent is an agent (other than tobacco) useful to supplement a subject's diet. The following are examples of useful nutritional agents: a vitamin; a mineral; a herb or other botanical; an amino acid; a dietary substance for use by man to supplement the diet by increasing the total dietary intake; or a concentrate, metabolite, constituent, extract, or combination thereof. Nutritional agents may include, but are not limited to vitamins, minerals, nutrients, proteins, amino acids, sugars, anti-oxidants, phytochemicals, carbohydrates, fiber or fat. The vitamin may be thiamine, vitamin A, vitamin B, vitamin C, vitamin D, vitamin E, vitamin F, niacin, biotin, vitamin K, panththenic acid, folate, riboflavin, or vitamin B6. The fat may be omega-3 or omega 6 fatty acid, alpha-linolenic acid or linolenic acid. The minerals may be calcium, chlorine, magnesium, phosphorus, potassium, sodium, sulphur, cobalt, copper, chromium, iodine, iron, manganese, molybdenum, selenium, vanadium or zinc.

Cosmetic Agents

In some embodiments the agent is a cosmetic agent. The cosmetic agent is an agent that can be rubbed, poured, sprinkled, or sprayed on, introduced into, or otherwise applied to the human body for cleansing, beautifying, promoting attractiveness, or altering the appearance. Cosmetic agents can include, but are not limited to, skin moisturizers, perfumes, lipsticks, fingernail polishes, eye and facial makeup preparations, cleansing shampoos, permanent waves, hair colors, and deodorants, as well as any substance intended for use as a component of such agents. The cosmetic agent may be an agent for improving the odour of the body of the subject in one or more locations. The cosmetic agent may be an agent for improving the appearance of the skin e.g. vitamin E or hyaluronic acid. The cosmetic agent may be an agent for improving hair quality. It will be appreciated that at least according to come embodiments, the encapsulated agent preparations may be deliverable topically or provided in topical compositions.

Food or Food Ingredients

In some embodiments the agent is food or a food ingredient. “Food” refers to materials taken into the body by mouth which provides nourishment in the form of energy or in the building of tissues. “Food ingredient” refers to any ingredient added to food, and includes, but is not limited to preservatives, emulsifiers, stabilizers, acids, non-stick agents, dyes, humectants, firming agents, antifoaming agents, colourings and flavourings, solvents and nutritive materials such as minerals, amino acids, vitamins and sugars.

Vesicles

In some embodiments the agent or agent preparation can be a vesicular structure, such as a liposome, transferosome, ethosome, microsphere, biodegradeable polymer, polymerized microparticle or polymerized nanoparticle. The vesicular structure can be encapsulated by the membrane composition.

Microscale and Nanoscale Medical Devices

In some embodiments the agent is a medical device. The agent may be a microscale or nanoscale medical device encapsulated within the membrane composition. The microscale or nanoscale medical device may be an instrument, apparatus, implement, machine, implant, in vitro reagent, or other similar or related article, including any component, part, or accessory, which is sized at a microscale or nanoscale and is useful in the diagnosis of disease or other conditions, or in the cure, mitigation, treatment, or prevention of disease, or can affect the structure or any function of the body. The membrane composition is useful to encapsulate a microscale or nanoscale medical device. The medical device may be suitable for diagnostics, long term monitoring or therapeutic treatment. In an embodiment, the membrane composition encapsulates a nanoshell.

### Methods of Making Encapsulated Particles

The present invention also provides methods for making an agent preparation encapsulated within the membrane composition as described herein.

In an embodiment, a method for making particles of an agent preparation encapsulated within the membrane composition can comprise the steps of:

a) combining the agent preparation with a liquid in which the agent preparation is immiscible or insoluble;

(b) subjecting the liquid and agent preparation to mixing effective to form particles of the agent preparation in the liquid; and

(c) adding the membrane composition to the agent preparation at a time during or after the subjecting step (b) to encapsulate each particle of the agent preparation with the membrane composition wherein at least a portion of the hydrophilic domain is present on the outer surface.

An embodiment of the above method is shown in FIG. 7. In step (a) the agent preparation is combined with a liquid (1) that is immiscible with the agent preparation or in which the agent preparation is insoluble such that under mixing the agent preparation (2) forms particles in the liquid. The agent or agent preparation can exist or be prepared such that it is generally hydrophilic or alternatively generally hydrophobic, as described herein, which enables the selection of a liquid with a contrasting HLB value such that the agent preparation is either insoluble or immiscible within the selected liquid. For example, a biphasic system can be established whereby application of mixing distributes the agent preparation into agglomerated portions (3) of agent preparation material, which are referred herein as “particles”. For example, the liquid (1) and the agent preparation (2) can form a biphasic system or a biphasic suspension. In step (b) the liquid (1) and agent preparation (2) can be subjected to mixing effective to form particles (3) of the agent preparation in the liquid. An individual particle (3) can have a composition, or be composed of, both the agent and excipient, if the excipient is present in the agent preparation. The membrane composition can then be added in step (c) to the agent preparation at a time during and/or after the subjecting step (b), to encapsulate the agent preparation within the membrane composition. An encapsulated particle (5) is formed having membrane layer encapsulating a portion of isolated agent preparation, which as mentioned is herein referred to as a “particle”. The membrane layer encapsulates the agent preparation such that at least a portion of the hydrophilic domain of the ganglioside is present on the outer surface. The membrane composition can therefore be used to form individually encapsulated particles wherein each particle has a composition comprising both an agent and excipient.

In an embodiment, the mixing includes sufficient shear to form particles. In an embodiment, mixing occurs for about 5 to 10, or 10 to 20, 20 to 30, or 30 to 40, or 40 to 50, or 50 to 60, or 60 to 70, or 70 or 80, or 80 to 100, or 100 to 120, or 120 to 140, or 140 to 160, or 160 to 180, or 180 to 200 seconds. In an embodiment, mixing occurs for about 1, 2, 3, 4, 5, 6 7, 8. 10, 15, 20, or 30 minutes. In an embodiment, the mixing has a shear rate of from about 500 to 100,000 s⁻¹. In an embodiment, mixing has a shear rate of at least 500 s⁻¹, or at least 1,000, at least 2,000 s⁻¹, or at least 5,000 s⁻¹, or at least 10,000 s⁻¹, or at least 15,000 s⁻¹, or at least 20,000 s⁻¹, or at least 30,000 s⁻¹. In an embodiment, mixing occurs in a blender. In an embodiment, mixing occurs in a high sheer mixer. In an embodiment mixing occurs in a Silverson L5 mixer.

In an embodiment of the above method, the agent preparation is combined in step a) with a liquid in which the agent preparation is insoluble and step (b) involves subjecting the liquid and agent preparation to mixing effective to form a biphasic suspension of particles of the agent preparation in the liquid. The use of a biphasic suspension can facilitate reduction in the formation of aggregated particles in embodiments where individual particles are desired.

In an embodiment, after step (c) the particles can be allowed to at least partially settle before further membrane composition is added, optionally with one or more other additives, for example with a glucose solution. The liquid in which the agent preparation is immiscible or insoluble may then be removed.

FIG. 7 provides a diagrammatic representation of one example or embodiment of the above method. The liquid (1) is selected such that the agent preparation (2) is immiscible or insoluble in the liquid. When the liquid (1) is immiscible with the agent preparation (e.g. when the agent preparation is a fluid such as a gel or liquid), then combining the liquid (1) with the agent preparation (2) can form two immiscible layers (step (a), FIG. 7). When the agent preparation is insoluble in the liquid, then combining the liquid with the agent preparation can form a biphasic suspension (not shown). Under shear mixing (step (b), FIG. 7), individual or discrete particles (i.e. portions of agent preparation material) are formed. It will be appreciated that an individual or discrete particle (3) is a portion of matter that can comprise the composition of the agent preparation (i.e. both agent and excipient when present). In step (c) the addition of the membrane composition can then independently encapsulate (e.g. coat or seal) each particle to provide a plurality of individually encapsulated particles.

In an embodiment, at least 20% of the agent in the agent preparation from step (a) is encapsulated in the membrane composition in step (c). In further embodiments, at least 30%, at least 40%, at least 50%, at least 60%, at least 70%, at least 80%, or at least 90%, of the agent in the agent preparation is encapsulated within the membrane composition.

Hydrophilic Particles

It will be appreciated that in embodiments where the agent or agent preparation is generally hydrophilic, then the immiscible or non-solubilising liquid can be selected to be generally hydrophobic. For example, a hydrophilic particle composed of the hydrophilic agent preparation may be formed in hydrocarbon based oil by application of shear mixing and subsequently encapsulated by the addition of membrane composition. It will be appreciated that the membrane composition can encapsulate one or more particles within one or more membrane composition layers wherein each layer comprises a ganglioside and lipid.

In embodiments where the agent or agent preparation is hydrophilic, the first layer is a bilayer (see FIG. 6). The membrane composition may comprise one or more further bilayers comprising the lipid and the hydrophilic domain of the ganglioside (not shown). The hydrophilic particles may be hydrophilic microparticles as described herein.

The hydrophilic agent preparation may comprise one or more excipients that are hydrophilic. The hydrophilic agent preparation may comprise an agent that is hydrophilic. The hydrophilic agent preparation, or particles thereof comprising both agent and excipient in the same particle, can be hydrophilic as described herein, for example having an HLB greater than about 10, or as otherwise described herein. It will be appreciated that hydrophobic agents may also be incorporated into a hydrophilic agent preparation to provide hydrophilic particles, for example by providing an effective amount of excipients that are hydrophilic.

The hydrophilic agent preparation, or agent and/or excipient thereof, can have an HLB above about 10, 11, 12, 13, 14, or 15, or as otherwise described herein. In various embodiments, the hydrophilic agent preparation, or agent and/or excipient thereof, can have an HLB between about 10 and 20, between about 11 and 20, between about 12 and 20, between about 13 and 20, or between about 14 and 20. Reference to a “hydrophilic agent preparation”, “hydrophilic agent” or “hydrophilic excipient” used herein can refer to any embodiment of the HLB value as described above.

The hydrophobic liquid may have an HLB below about 9, 8, 7, 6, 5, 4, or 3, or as otherwise described herein. In various embodiments, the hydrophobic liquid may have an HLB between about 0 and 10, between about 0 and 8, between about 0 and 6, or between about 0 and 4.

Particles formed from a hydrophilic agent preparation, including agent or excipient, as described above, can be referred to herein as hydrophilic particles. In one embodiment, a hydrophilic particle comprises one or more bilayers of the membrane composition encapsulating the hydrophilic agent preparation. In another embodiment, the hydrophilic particles comprise two or more bilayers of the membrane composition. For example, a first bilayer can encapsulate the hydrophilic particles, with a second bilayer encapsulating the first bilayer. In another embodiment, a first bilayer encapsulates the hydrophilic particles, with a second bilayer encapsulating the first bilayer, wherein an intervening layer comprising a hydrocarbon solvent exists between the first and second bilayers. It will be appreciated that the intervening layer does not comprise a ganglioside. The hydrocarbon solvent can be a saturated hydrocarbon such as an alkane, unsaturated hydrocarbons such as an alkene, a cycloalkane such as cyclohexane, or aromatic hydrocarbon. In an embodiment, the hydrocarbon solvent is a straight chain or branched C₅₋₂₀alkyl. In another embodiment, the hydrocarbon solvent is hexane, heptane, octane, nonane, decane, undecane, dodecane, or a combination thereof. In another embodiment, the hydrocarbon solvent is heptane.

The agent preparation can comprise an excipient. As described above, a hydrophilic agent preparation can comprise a hydrophilic excipient. The excipient, or hydrophilic excipient, can be a gelling agent. The gelling agent may be a hydrophilic pH sensitive gelling agent, for example having an HLB above about 10. The pH sensitive gelling agent may be a gel stable at a pH below about 3. The pH sensitive gelling agent may be pectin or modified pectin as described herein.

In another embodiment, there is provided a method for making particles of an agent preparation encapsulated within the membrane composition, wherein the method comprises the steps of:

a) combining a hydrophilic agent preparation with a hydrophobic liquid in which the agent preparation is immiscible or insoluble such that under mixing the agent preparation forms particles in the liquid;

(b) subjecting the liquid and agent preparation to mixing effective to form particles of the agent preparation in the liquid; and

(c) adding the membrane composition to the agent preparation at a time during or after the subjecting step (b) to encapsulate each particle of the agent preparation with the membrane composition wherein at least a portion of the hydrophilic domain is present on the outer surface of the membrane composition.

Subjecting the agent preparation to mixing can be effective to form particles of the agent preparation. The mixing can be effective to form microparticles of the agent preparation. For example, the size of the microparticles may be provided by diameters (in μm) ranging from about 0.01 to 10, 0.1 to 7, 0.2 to 6, 0.3 to 5, 0.4 to 4, or 0.5 to 3. The size of the microparticles may be provided by diameters (in μm) of less than about 10, 5, 4, 3, 2, or 1. The size of the microparticles may be provided by diameters (in μm) of greater than about 0.01, 0.05, 0.1, 0.2, 0.3, 0.4, 0.5, 0.6, 0.7, 0.8, 0.9, 1, or 2. The particle size may be measured by methods known in the relevant art, for example, by light microscopy.

The hydrophilic agent can for example be a pharmaceutical substance, a virus, a nanoparticle, a cell or a T-cell. The hydrophilic agent may be insulin, glucagon, GLP (glucagon like protein) and its analogs, calcitonin, parathyroid hormone, relaxin, interferon or a growth hormone.

The agent may be dissolved or dispersed in an excipient that is an aqueous gel. The amount of aqueous gel can be sufficient for “wetting” the agent. The aqueous gel may comprise any commercially available thickening or gelling agent, such as thickening or gelling agent that is suitable for oral administration. Examples of thickening or gelling agents that are suitable for oral administration include, but are not limited to, acetylated distarch adipate, agar, alginic acid, arrowroot, beta-glucan, calcium alginate, carrageenan, cassia gum, chondrin, collagen, corn starch, dextrin, erythronium japonicum, fumed silica, galactomannan, gelatin, gellan gum, glucomannan, guar gum, gulaman, gum karaya, hydroxypropyl distarch phosphate, hypromellose, irvingia gabonensis, konjac, kudzu, locust bean gum, methyl cellulose, millet jelly, modified starch, monodora myristica, mung bean, natural gum, pectin, phosphated distarch phosphate, polydextrose, potato starch, psyllium seed husks, roux, sago, salep, starch, tapioca, tragacanth, waxy corn, xanthan gum, glycogen and pre-gelatinised starches.

The gel can be provided at a temperature above the setting temperature, and then the agent added. Further cooling may be applied followed by a sufficient time to allow enhancement of gel strength.

The hydrophilic excipient or agent preparation may be a hydrophilic liquid that can be cooled before it is dispersed in the hydrophobic liquid. The hydrophilic liquid may be dispersed in at least twice the volume of a hydrophobic liquid, for example it may be dispersed in at least 3, 4 or 5 times the volume of a hydrophobic liquid.

The hydrophobic liquid may be any hydrophobic liquid known in the relevant art provided it is not be capable of dissolving or acting as a solvent for the hydrophilic agent. The hydrophobic liquid may be a C₃₋₁₀hydrocarbon, for example a C₃₋₁₀alkane. The hydrophobic liquid may be pentane, hexane or heptane or another alkane in which the agent is insoluble.

The hydrophilic excipient or hydrophilic agent preparation may be a gel or a solid. The gel may be an aqueous gel, for example a gel comprising pectin. The aqueous gel may comprise the hydrophilic agent, which can be dissolved or dispersed in the aqueous gel by heating the aqueous gel to a temperature greater than its gelation temperature such that it forms a liquid aqueous gel having a viscosity of for example less than about 100 milliPascal seconds. The hydrophilic agent may be added, with stirring, to the aqueous gel when the aqueous gel is at a temperature ranging from about 50° C. and about 70° C., for example at a temperature of about 60° C. The aqueous gel may comprise pectin, a sugar, water and acid to adjust the pH. The sugar may be xylitol or glucose. The acid may be tartaric acid.

The mixture of the hydrophilic agent and aqueous gel may be frozen and pulverized, e.g., freezing the mixture, optionally under reduced pressure removing at least some of the water from the gel; and then reducing the particle size of the frozen mixture, e.g., by grinding, milling or pulverizing it into a powder. The particle size of the frozen mixture may be sized according to any embodiments as described herein for the particle size of the “particles”.

The hydrophilic agent preparation may be allowed to set for a period of time, e.g., from about 8 to about 24 hours, before freezing. The agent preparation may be frozen at a temperature of about −80° C. The hydrophobic liquid may be cooled to a temperature of about −80° C. before the hydrophilic agent added thereto. The composition comprising the hydrophilic agent may be freeze-dried to provide dry product.

The hydrophobic liquid may be removed as described above, or by concentration in vacuo, e.g., using a rotary evaporator and/or by adding an aqueous medium, for example an aqueous medium comprising an equal volume of 1% tartaric acid or a derivative thereof and 20% sugar alcohol in water. The sugar alcohol may be xylitol, sorbitol or glyercol.

Two or more of steps (a), (b) and (c) may be performed concurrently.

The method for making particles of an agent preparation encapsulated within the membrane composition may further comprise one or both of:

(d) contacting the encapsulated agent preparation with a hydrophobic liquid; and

(e) adding additional membrane composition to the encapsulated agent preparation.

Without being bound by theory, step (e) may serve to further encapsulate the agent preparation with the membrane composition or to encapsulate any agent preparation that is not encapsulated during step (c). In some embodiments, where the additional membrane composition further encapsulates the agent preparation, the hydrophobic liquid is present between two membrane composition layers, each being a layer or bilayer.

Where the hydrophobic liquid is present between the two bilayers of membrane composition (see FIG. 4b), the hydrophobic liquid may be removed, e.g., under reduced pressure, while retaining the two bilayers of membrane composition. It will be appreciated that one or both of steps (d) and (e) may be repeated to provide further layers of membrane composition around the particles.

Hydrophobic Particles

It will be appreciated that in embodiments where the agent or agent preparation is generally hydrophobic, then the immiscible or non-solubilising liquid can be selected to be generally hydrophilic. For example, a hydrophobic particle composed of the hydrophobic agent preparation may be formed in water by application of mixing and subsequently encapsulated by the addition of membrane composition. It will be appreciated that the membrane composition can encapsulate one or more particles within one or more membrane composition layers wherein each layer comprises a ganglioside and lipid.

In embodiments where the agent or agent preparation is hydrophobic, the first layer is a monolayer (see 3b in FIG. 5). The membrane composition may comprise one or more further bilayers comprising the lipid and the hydrophilic domain of the ganglioside (not shown). It will be appreciated that the monolayer, or each bilayer thereon comprises a composition including gangliosides and lipids.

The hydrophobic agent preparation, or agent and/or excipient thereof, can have an HLB below about 9, 8, 7, 6, 5, 4, or 3, or as otherwise described herein. In various embodiments, the hydrophobic agent preparation, or agent and/or excipient thereof, can have an HLB between about 0 and 10, between about 0 and 8, between about 0 and 6, or between about 0 and 4.

The hydrophilic liquid can have an HLB above about 10, 11, 12, 13, 14, or 15, or as otherwise described herein. In various embodiments, the hydrophilic liquid can have an HLB between about 10 and 20, between about 11 and 20, between about 12 and 20, between about 13 and 20, or between about 14 and 20.

In another embodiment, there is provided a method for making particles of an agent preparation encapsulated within the membrane composition, wherein the method comprises the steps of:

a) combining a hydrophobic agent preparation with a hydrophilic liquid in which the agent preparation is immiscible or insoluble;

(b) subjecting the liquid and agent preparation to mixing effective to form particles of the agent preparation in the liquid; and

(c) adding the membrane composition to the agent preparation at a time during or after the subjecting step (b) to encapsulate each particle of the agent preparation with the membrane composition wherein at least a portion of the hydrophilic domain is present on the outer surface.

The mixing in step (b) can be effective to form particles of the hydrophobic agent preparation, wherein the particles have diameters (in μm) of between about 0.1 to 20, 0.2 to 15, 0.3 to 10, 0.5 to 5, or 1 to 3. The mixing can be effective to form particles of the hydrophobic agent preparation having particle diameters (in μm) of less than about 15, 10, 5, 4, 3, 2, or 1. The mixing can be effective to form particles of the hydrophobic agent preparation having particle diameters (in μm) of greater than about 0.2, 0.3, 0.4, 0.5, 0.6, 0.7, 0.8, 0.9, 1, 2, or 3. The particle size can be measured by methods known in the relevant art, for example, by light microscopy.

The hydrophobic agent may be insoluble or practically insoluble in water. The hydrophobic agent may be combined with the hydrophilic liquid without modification. The hydrophobic agent may be dissolved or dispersed in an oil before it is combined with the hydrophilic liquid. The hydrophobic agent may be dispersed or dissolved in an oil when, for example, the hydrophobic agent is a solid. A hydrophobic agent which is a solid (e.g. powder) may be used in that form. If the hydrophobic agent is oily at room temperature, it may be dissolved or dispersed in an oil before it is combined with the hydrophilic liquid.

The hydrophobic agent preparation comprising a hydrophobic agent may be an oil. Any oils, or liquid fats, can be used without limitation, e.g. animal oils, marine oils and vegetable oils. The oil may be a vegetable oil, that is, a triglyercide extracted from a plant. Examples of vegetable oils include, but are not limited to, almond oil, ambadi seed oil, argan oil, avocado oil, canola oil, cashew oil, castor oil, coconut oil, colza oil (toxic oil syndrome), corn oil, cottonseed oil, grape seed oil, hazelnut oil, hemp oil, linseed oil (flaxseed oil), macadamia oil, marula oil, mongongo nut oil, mustard oil, olive oil, palm oil, palm kernel oil, peanut oil, pecan oil, perilla oil, pine nut oil, pistachio oil, poppyseed oil, pumpkin seed oil, rapeseed oil, rice bran oil, safflower oil, sesame oil, soybean oil, sunflower oil, tea seed oil, walnut oil and watermelon seed oil. The oil may also be rice bran oil. The oil may be a fish oil. The oil may also be an animal liquid fat derived from lard or shortening.

The hydrophilic liquid may be any hydrophilic liquid known in the relevant art providing it is not be capable of dissolving or acting as a solvent for the hydrophobic agent, excipient or hydrophobic agent preparation. The hydrophilic liquid may be an aqueous solution, for example a lactose solution, or water. The hydrophilic liquid may have a hydrophilic-lipophilic balance (HLB) of above about 10, 11, 12, 13, 14, or 15, or as otherwise described herein.

### Pharmaceutical Formulations and Compositions

The present invention also provides pharmaceutical compositions for veterinary or human medical use. The pharmaceutical compositions can comprise the agent preparation, for example wherein the agent preparation comprises a therapeutic concentration of a therapeutic agent, encapsulated within the membrane composition according to any embodiments as described herein and pharmaceutically acceptable excipients. The pharmaceutical composition may be a pharmaceutical formulation comprising a pharmaceutically acceptable carrier.

The carrier(s) or excipients for the formulations or compositions should be pharmaceutically acceptable in the sense of being compatible with the other ingredients of the formulation and not unduly deleterious to the recipient thereof.

The compositions may also include polymeric excipients/additives or carriers, e.g., polyvinylpyrrolidones, derivatised celluloses such as hydroxymethylcellulose, hydroxyethylcellulose, and hydroxypropylmethylcellulose, ficolls (a polymeric sugar), hydroxyethylstarch (HES), dextrates (e.g., cyclodextrins, such as 2-hydroxypropyl-β-cyclodextrin and sulfobutylether-β-cyclodextrin), polyethylene glycols, and pectin. The compositions may further include diluents, buffers, binders, disintegrants, thickeners, lubricants, preservatives (including antioxidants), flavoring agents, taste-masking agents, inorganic salts (e.g., sodium chloride), antimicrobial agents (e.g., benzalkonium chloride), sweeteners, antistatic agents, sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). Other pharmaceutical excipients and/or additives suitable for use in the compositions are listed in “Remington: The Science & Practice of Pharmacy”, 19.sup.th ed., Williams & Williams, (1995), and in the “Physician's Desk Reference”, 52.sup.nd ed., Medical Economics, Montvale, N.J. (1998), and in “Handbook of Pharmaceutical Excipients”, Third Ed., Ed. A. H. Kibbe, Pharmaceutical Press, 2000.

In an embodiment, the composition is adapted for enteral or parenteral administration. Enteral administration includes oral or rectal administration. The encapsulated particles, or any embodiments thereof as described herein, may be formulated in compositions including those suitable for oral, rectal, topical, transdermal, mucosal, nasal, inhalation to the lung, by aerosol, ophthalmic, or parenteral (including intraperitoneal, intravenous, subcutaneous, or intramuscular injection) administration. The compositions may conveniently be presented in unit dosage form and may be prepared by any of the methods well known in the art of pharmacy. In general, the compositions can be prepared by bringing the encapsulated particles into association with a liquid carrier to form a solution or a suspension, or alternatively, bringing the encapsulated microparticles into association with formulation components suitable for forming a solid, optionally a particulate product, and then, if warranted, shaping the product into a desired delivery form. Solid formulations, when particulate, may comprise particles for excipients other than the encapsulated particles (whose particle size embodiments have been previously described above) with sizes ranging from about 1 nanometer to about 500 microns. The composition may contain excipients that are nanoparticulates having a particulate diameter of below about 1000 nm, for example, between about 5 and about 1000 nm, about 5 and about 500 nm, about 5 to about 400 nm, such as about 5 to about 50 nm and for example between about 5 and about 20 nm. The encapsulated particles may be mono- or poly-dispersed in the composition, with PDI of ranging from about 1.01 to about 1.8, from about 1.01 to about 1.5, or about 1.01 to about 1.2.

Compositions of the present invention suitable for oral administration may be provided in any conventional oral dosage forms, such as solids, semi-solids and liquids. Discrete units may include pills, capsules, cachets, tablets, lozenges, films, powders, solutions, pastes and the like, each containing a predetermined amount of the agent (encapsulated within microparticles) as a powder or granules; or a suspension in an aqueous liquor or non-aqueous liquid such as a syrup, an elixir, an emulsion, a draught, and the like. The compositions and formulations may also be provided with other agents in addition to those contained in the encapsulated particles.

A tablet may be made by compression or molding, optionally with one or more accessory ingredients. Compressed tablets may be prepared by compressing in a suitable machine, with the encapsulated particles being in a free-flowing form such as a powder or granules which is optionally mixed with a binder, disintegrant, lubricant, inert diluent, surface active agent or dispersing agent. Molded tablets comprised with a suitable carrier may be made by molding in a suitable machine.

A syrup may be made by adding the encapsulated particles to a concentrated aqueous solution of a sugar, for example sucrose, to which may also be added any accessory ingredient(s). Such accessory ingredients may include flavorings, suitable preservatives, an agent to retard crystallization of the sugar, and an agent to increase the solubility of any other ingredient, such as polyhydric alcohol, for example, glycerol or sorbitol.

Formulations suitable for parenteral administration may comprise a sterile aqueous preparation of the encapsulated particles, which can be formulated to be isotonic with the blood of the recipient.

Nasal formulations may comprise purified aqueous solutions of the encapsulated particles with preservative agents and isotonic agents. Such formulations are preferably adjusted to a pH, microparticle size and isotonic state compatible with the nasal mucous membranes.

Formulations for rectal administration may be presented as a suppository with a suitable carrier such as cocoa butter, or hydrogenated fats or hydrogenated fatty carboxylic acids.

Ophthalmic formulations may be prepared by a similar method to the nasal spray, except that the pH and isotonic factors are preferably adjusted to match that of the eye.

Topical formulations may comprise the encapsulated particles dissolved or suspended in one or more media such as mineral oil, petroleum, polyhydroxy alcohols or other bases used for topical formulations. The addition of other accessory ingredients as noted above may be desirable.

Pharmaceutical formulations may also be provided which are suitable for administration by inhalation including as an aerosol. These formulations may comprise a solution or suspension of the encapsulated particles. The desired formulation may be placed in a small chamber and nebulized. Nebulization may be accomplished by compressed air or by ultrasonic energy to form a plurality of liquid droplets or solid particles comprising the encapsulated particles.

Often drugs are co-administered with other drugs in combination therapy. The encapsulated particles may therefore be administered as combination therapies.

In some embodiments, the encapsulated particles may be formulated for topical or transdermal delivery such as an ointment, a lotion or in a transdermal patch or use of microneedle technology.

The encapsulated particles may also be used to provide controlled or targeted release of the agents, including slow-release. In slow-release formulations, the formulation ingredients are selected to release the encapsulated particles from the formulation over a prolonged period of time, such as days, weeks or months. This type of formulation includes transdermal patches or in implantable devices that may be implanted surgically, deposited subcutaneously or by injection intraveneously, subcutaneously, intramuscularly, intraepidurally, intracranially or intrathecally.

Bioavailability of the agent in an oral composition may be at least about 0.1%, or at least about 0.5%, or at least about 1%, or at least about 2%, or at least about 3%, or at least about 4%, or at least about 5%, or at least about 10%, or at least about 15%, or at least about 20%, or at least about 25%, or at least about 30%, or at least about 35%, or at least about 40%, or at least about 50%, or at least about 60%, or at least about 70%, or at least about 80%, or at least about 90%.

In an embodiment, at least about 0.1%, at least about 0.2%, or at least about 0.4%, or at least about 0.5%, or at least about 1%, or at least about 2%, or at least about 3%, or at least about 4%, or at least about 5%, or at least about 10%, or at least about 15%, or at least about 20%, or at least about 30% of the encapsulated microparticles in an oral composition may cross the blood brain barrier. In an embodiment, at least about 0.2%, or at least about 0.4%, or at least about 0.5%, or at least about 1%, or at least about 2%, or at least about 3%, or at least about 4%, or at least about 5%, or at least about 10%, or at least about 15%, or at least about 20%, or at least about 30% of the agent in an oral composition may cross the blood brain barrier.

In an embodiment, an oral composition may provide a serum or plasma concentration of at least about 0.1 to about 100 μg/mL, or at least about 10 to about 250 μg/mL, or at least about 20 to about 400 μg/mL, or at least about 100 to about 1000 μg/mL, or at least about 1000 to about 5000 μg/mL of the agent.

### Delivery to or via the Lymphatic System

There is also provided a method of delivering an agent to or via the lymphatic system of a subject comprising administering to the subject the agent preparation encapsulated within a membrane composition according to any embodiments thereof as described herein, wherein the agent preparation encapsulated within the membrane composition is internalized into the subject by absorption across the subject's gastrointestinal epithelial barrier.

The absorption may be by transcytosis. There is also provided a method of delivering an agent to or via the lymphatic system of a subject comprising administering to the subject the agent preparation encapsulated within a membrane composition according to any embodiments thereof as described herein, wherein the agent preparation encapsulated within the membrane composition is internalized into the subject by transcytosis across the subject's gastrointestinal epithelial barrier.

The composition can be administered orally. The agent can be released from the encapsulated agent preparation or individually encapsulated particles thereof in the lymphatic system. The agent can be released from the encapsulated agent preparation or individually encapsulated particles thereof in the blood circulatory system, or can be released in the lymphatic system and thereafter enter the blood circulatory system. The encapsulated agent preparation or individually encapsulated particles thereof can be targeted to a predetermined cell, tissue or organ in the subject by a species of gangliosides present on the surface of the encapsulated agent preparation or individually encapsulated particles thereof. The agent can be released near, on or in such predetermined cell, tissue or organ.

The “lymphatic system” includes the system of lymphatic vasculature (the lymphatic vessels), including lacteals, the gut associated lymphoid tissue (GALT), the lymph nodes, spleen and thymus. The lymphatic system plays a role in developing immune responses and draining interstitial fluid form the body's tissues and returning it to the heart via e.g. at the junction between the thoracic duct and the subclavian vein. Fluid transported in the lymphatic vasculature is known as lymph fluid. The vessels of the lymphatic system carry and direct the lymph to the heart via the venus blood stream where it is delivered to the circulatory system or cardiovascular system (i.e. the systemic blood circulatory system).

Encapsulated particles delivered to the lymphatic system can be directed to the blood circulatory system via the lymphatic vasculature (e.g. the subclavian veins). This is advantageous as delivery to the lymphatic system bi-passes the hepatic portal venus system and delivery to the liver where such agents and microparticles are often degraded before reaching the blood circulatory system.

“Transcytosis” refers to a type of transcellular transport in which encapsulated particles can be transported across the interior of a cell, for example the encapsulated microparticles can be captured in vesicles on one side of the cell by endocytosis, transported across the cell, and ejected intact on the other side by exocytosis. The encapsulated microparticles may be internalized intact into the subject by transcytosis across the subject's epithelial barrier.

“Endocytosis” also referred to as “endocytic uptake” may be receptor mediated or may be non-receptor mediated. In an embodiment, the receptor mediated endocytosis may be lipid mediated endocytosis. In an embodiment, the receptor mediated endocytosis may be clathrin-mediated endocytosis. The endocytosis may be provided by encapsulated particles having particle diameters (in μm) of between about 0.1 to about 20, about 0.2 to about 15, about 0.3 to about 10, about 0.5 to about 5, or about 1 to about 3. The endocytosis may be provided by encapsulated particles having particle diameters (in μm) of less than about 20, about 15, about 10, about 5, about 4, about 3, about 2, or about 1. The endocytosis may be provided by encapsulated particles having particle diameters (in urn) of greater than about 0.1, about 0.2, about 0.3, about 0.4, about 0.5, about 0.6, about 0.7, about 0.8, about 0.9, about 1, about 2, or about 3. In an embodiment, encapsulated particles of up to at least about 50 nm, or at least about 100 nm, or at least about 150 nm, or at least about 200 nm in diameter may be endocytosed by clathrin-mediated endocytosis. In an embodiment, the endocytosis may be phagocytosis. In an embodiment, encapsulated particles of at least about 50 nm, at least about 100 nm, at least about 200 nm, at least about 500 nm, at least about 1000 nm, or at least about 5000 nm, or at least about 10,000 nm, or at least about 20,000 nm are endocytosed by phagocytosis. Although not wishing to be bound by theory, it is believed that the gangliosides in the membrane compositions may provide, facilitate or trigger the endocytosis of the encapsulated particles.

“Internalized into the subject by transcytosis” refers to uptake of encapsulated particles from the environment external to the subject by a cell of the epithelial barrier and transport of the encapsulated particles intact across the cell of the epithelial barrier into the internal environment of the subject by transcytosis i.e. endocytosis of the encapsulated particles on the apical membrane of the cell, transport across the cytoplasm of the cell, and exocytosis of the encapsulated particles from the basolateral membrane of the cell.

The term “epithelial barrier” refers to the layer of cells that lines the outer surface or surface of a cavity which is internal to a subject and serves as a barrier between the internal environment of a subject and the environment external to the subject. The epithelial barrier may be the gastrointestinal epithelial barrier. The gastrointestinal epithelial barrier may be the intestinal epithelial barrier which lines the small and/or large intestine. The intestinal epithelial barrier may be the epithelium which lines the duodenum. The intestinal epithelial barrier may be the epithelium which lines the jejunum. The intestinal epithelial barrier may be the epithelium which lines the ileum. The intestinal epithelial barrier may be the epithelium which lines the colon. The environment external to the subject may be the lumen of the gastrointestinal tract.

The term “gastrointestinal” refers to the mouth, oesophagus, stomach, small intestine (duodenum, jejunum and ileum) and large intestine (colon and rectum). Thus, a gastrointestinal epithelial cell can be an epithelial cell lining the mouth, oesophagus, stomach, small intestine or large intestine.

The encapsulated particles may be internalized into the subject by transcytosis across the subject's epithelial barrier and are delivered to the lymphatic system. The encapsulated particles may be transported via the lymphatic vasculature to the venus vasculature (e.g. at the junction between the thoracic duct and the subclavian vein) where they will be delivered to the cardiovascular system. This allows for systemic delivery of the encapsulated particles to the blood circulatory system and/or to a tissue or organ of the subject. In an embodiment, the encapsulated particles can cross the blood brain barrier. In an embodiment, the contents of the encapsulated particles may be released in a cell, tissue or organ of the subject. In an embodiment, the contents of the encapsulated particles may be released in the blood stream. The agent may be released from the encapsulated particles in the lymph node. This is advantageous where the agent is a cell or an antigen which stimulates an immune response (i.e. where the encapsulated particles are used to deliver a vaccine composition or immunotherapy agent to a subject). For release of the agent from the encapsulated particles in the lymph node the encapsulated particles can comprise a pH sensitive gelling agent which aids in release of the biologically agent in the lymph node. In an embodiment, the pH sensitive gelling agent is pectin or modified pectin. Once released from the encapsulated particles, the biologically agent can be transported via the lymphatic vasculature to the cardiovascular system. This allows for systemic delivery of the agent to a tissue or organ of the subject.

The present invention provides for targeted delivery of encapsulated particles to pre-determined cells, tissues or organs. Species of gangliosides may be pre-selected and used in membrane compositions for encapsulating particles for facilitating delivery of the intact encapsulated particles to pre-determined cells, tissues or organs. For example, the gangliosides in the membrane composition may provide multiple properties that facilitate targeted delivery, such as protecting the particles (comprising an agent) from enzymatic degradation, facilitating transcytosis and enhancing binding and accumulation at or near pre-determined cells, tissues or organs. For example, the encapsulated particles can be orally administered and travel through the lymphatic system, into the blood circulatory systems, and accumulate at or near pre-determined cells, tissues or organs, for release of the agent from the encapsulated particles at that pre-determined site in the body. It will be appreciated that a targeted drug delivery system, such as that provided by the present invention, is particularly advantageous. It was surprisingly discovered that particular species or types of gangliosides of the membrane compositions can provide, facilitate or trigger targeting and delivery of agents to particular sites, for example GM1 ganglioside enabling targeting to the brain or GM3 ganglioside enabling targeting to adipose tissue.

In an embodiment, the encapsulated particles may be targeted to a predetermined cell, tissue or organ in the subject by incorporating a particular species of ganglioside into the membrane composition. Although not wishing to be bound by any theory, it is believed that each species of ganglioside recognizes and/or binds preferentially to a particular cell type. It has been shown that the ganglioside species GM1 may recognize and/or bind preferentially to, for example, neural cells. It has been shown that the ganglioside species GM3 may recognize and/or bind preferentially to, for example, adipocytes. The ganglioside species GM4, GD2, and GD4 may recognize and/or bind preferentially to, for example renal cells.

“Targeted” generally refers to enhancing delivery, binding and/or endocytosis and/or accumulation to, at or near pre-determined cells, tissues or organs. Following delivery, accumulation, endocytosis, or binding, of the encapsulated particles, the contents thereof may for example be released outside a targeted cell and/or the encapsulated particles may be internalized into the targeted cell where their contents are then released. The contents of the encapsulated particles may be released in the intracellular space of the tissue or the organ and/or may be released in the cells of the tissue or the organ.

In an embodiment, the encapsulated particles may be targeted to an astrocyte, neuronal cell, hepatocyte, renal cell, adipocyte, or myocardiocyte by incorporation of a particular species of ganglioside into the membrane composition and optionally by incorporation of a particular type of excipient (e.g. gelling agent) into the agent preparation or particle thereof. In an embodiment, the encapsulated particles may be targeted to cancerous or precancerous cell by incorporation of a particular species of ganglioside into the membrane composition and optionally by incorporation of a particular type of excipient (e.g. gelling agent) into the agent preparation or particle thereof. In an embodiment, the encapsulated particles may be targeted to adipose, renal or neural tissue by incorporation of a particular species of ganglioside into the membrane composition, for example GM3 or GD3 ganglioside, and optionally by incorporation of a particular type of excipient (e.g. gelling agent) into the agent preparation or particle thereof. In an embodiment, the encapsulated particles may be targeted to the brain, liver, kidney, adipose or heart by a species of ganglioside, by incorporation of a particular species of ganglioside into the membrane composition, for example GM1 ganglioside, and optionally by incorporation of a particular type of excipient (e.g. gelling agent) into the agent preparation or particle thereof.

The delivery may be to a neural cell, neural tissue or a brain by incorporating the species GM1 into the membrane composition. The delivery may be to an adipocyte or to adipose by incorporating the species GM3 or GD3 into the membrane composition. The delivery may be to a renal cell, renal tissue or a kidney by incorporating the species GM4, GD2, and GD4 into the membrane composition.

In an embodiment, the invention provides a method for delivering an agent to a predetermined cell, tissue or organ in a subject comprising administering to the subject the agent preparation encapsulated within a membrane composition as described herein or the pharmaceutical composition as described herein, by having present a portion of the hydrophilic domain of a species of ganglioside on the outer surface of the membrane composition.

In an embodiment, the invention provides a method for delivering an agent to a subject's neural cell, neural tissue or brain, comprising administering to the subject the agent preparation encapsulated within a membrane composition as described herein or the pharmaceutical composition as described herein, by having present a portion of the hydrophilic domain of the ganglioside GM1 or GM2 on the outer surface of the membrane composition.

In an embodiment, the invention provides a method for delivering an agent to a subject's adipocyte or adipose tissue, comprising administering to the subject the agent preparation encapsulated within a membrane composition as described herein or the pharmaceutical composition as described herein, by having present a portion of the hydrophilic domain of the ganglioside GM3 or GD3 on the outer surface of the membrane composition.

In an embodiment, the invention provides a method for delivering an agent to a subject's renal cell or kidney, comprising administering to the subject the agent preparation encapsulated within a membrane composition as described herein or the pharmaceutical composition as described herein, by having present a portion of the hydrophilic domain of the ganglioside GM4, GD2 or GD4, on the outer surface of the membrane composition.

There is also provided use of an encapsulated agent preparation or encapsulated particles thereof according to any embodiments thereof as described herein, for delivering an agent to the lymphatic system of a subject. The use may be in the manufacture of a medicament.

There is also provided an encapsulated agent preparation or encapsulated particles thereof according to any embodiments thereof as described herein, for use in delivering an agent to the lymphatic system of a subject.

The encapsulated agent preparation or encapsulated particles thereof may be internalized into the subject by transcytosis across the subject's epithelial barrier.

There is also provided use of the agent preparation encapsulated within the membrane composition according to any embodiments thereof as described herein, for delivering an agent to a subject's neural cell, neural tissue or brain, wherein the ganglioside in the membrane composition comprises a GM1 ganglioside. The use may be in the manufacture of a medicament.

There is also provided the agent preparation encapsulated within the membrane composition according to any embodiments thereof as described herein, for use in delivering an agent to a subject's neural cell, neural tissue or brain, wherein the ganglioside in the membrane composition comprises a GM1 ganglioside.

There is also provided use of the agent preparation encapsulated within the membrane composition according to any embodiments thereof as described herein, for delivering an agent to a subject's adipocyte or adipose tissue, wherein the ganglioside in the membrane composition comprises a GM3 or GD3 ganglioside. The use may be in the manufacture of a medicament.

There is also provided an agent preparation encapsulated within the membrane composition according to any embodiments thereof as described herein, for use in delivering an agent to a subject's adipocyte or adipose tissue, wherein the ganglioside in the membrane composition comprises a GM3 or GD3 ganglioside.

There is provided use of the agent preparation encapsulated within the membrane composition according to any embodiments thereof as described herein, for delivering an agent to a subject's renal cell or kidney, wherein the ganglioside in the membrane composition comprises a GM4, GD2 or GD4 ganglioside. The use may be in the manufacture of a medicament.

There is also provided agent preparation encapsulated within the membrane composition according to any embodiments thereof as described herein, for use in delivering an agent to a subject's renal cell or kidney, wherein the ganglioside in the membrane composition comprises a GM4, GD2 or GD4 ganglioside.

Method of Treatment

There is also provided a method for treating a disease or disorder, comprising administering to a subject in need thereof an effective amount of the agent preparation encapsulated within a membrane composition, or the pharmaceutical composition, according to any embodiments thereof as described herein.

The encapsulated particles may be suitable for treatment and or prevention of a disease and/or disorder. In an embodiment, the disease or disorder is neurological, inflammatory, urogenital, endocrine, lymphatic, lysosomal, digestive, cardiovascular disease, lymphatic respiratory or a musculoskeletal disease or disorder. In an embodiment, the disease is cancer. In an embodiment, the disease is an infectious disease. In an embodiment, the disorder is a lysosomal storage disorder.

### EXAMPLES

The present invention is further described by the following examples. It is to be understood that the following description is for the purpose of describing particular embodiments only and is not intended to be limiting with respect to the above description.

In some of the following examples surrogate dyes have been used to represent hydrophilic and hydrophobic agents. The use of surrogate dyes such as methylene blue, nile red, sodium fluorescein and red fluorescent protein in the following examples as supported at least by Zhai et al. 1996, Ozdemir et al. 2000, Aramwit et al. 2015, Sakai et al. 1997, Zderic et al 2004, Serrano et al. 2015, Gumpta et al. 2012, Prokop et al. 2014, Chakraborty et al. 2009.

### General Experimental

Experiments requiring anhydrous conditions were performed under a dry nitrogen or argon atmosphere using apparatus heated and dried under vacuum, unless stated otherwise.

### A. Preparation of Membrane Composition

### Example A1

Membrane composition extract was prepared using 500 g buttermilk powder extracted twice with 1000 mL isopropanol. The resultant extract was filtered (540 filter paper) and concentrated to 100 mL using a Rotovapor to provide a membrane composition comprising GM3 ganglioside.

### Example A2

A membrane composition was prepared using sheep's brain extract that was extracted with isopropanol. The resultant extract was filtered (540 filter paper) and concentrated to 100 mL using a Rotovapor to provide a membrane composition comprising GM1 ganglioside.

### B. Encapsulated Particles

### i. Preparation of Encapsulated Particles

### Example B1—Encapsulated Hydrophobic Agent Microparticles

Nile Red was used as a surrogate dye for a hydrophobic biologically agent. Nile Red (2.2 mg) in isopropanol (3 mL) was emulsified in virgin olive oil (200 mL). This mixture was then combined with an aqueous solution comprising 10% lactose (300 mL) and the resultant biphasic mixture was dispersed with high shear mixing for 60 seconds. The membrane composition (5 mL) from Example A1 comprising GM3 ganglioside was then added to produce GM3 encapsulated microparticles comprising olive oil and Nile Red.

### Example B2—Encapsulated Hydrophilic Agent Microparticles

Sodium fluorescein was used as a surrogate dye for a hydrophilic agent. An aqueous gel was prepared by dissolving gelatin (5.6 g) in water (200 mL). Sodium fluorescein was added to the aqueous gel which was then heated to a temperature of 80° C. and a viscosity of less than about 100 milliPascal seconds. The liquid was allowed to cool to a temperature of 50° C. before adding heptane (800 mL) and dispersing it under high shear mixing for bursts of about 30 seconds until the composition solidified. Membrane composition (2 mL) from Example A1 comprising GM3 ganglioside was then added. When the temperature of the mixture was cooled to about 20° C., 10% lactose (200 mL) was added and the microparticles were allowed to settle into the lactose phase. The heptane was then substantially removed, before adding pentane (50 mL) and a further amount of membrane composition from Example A1 (2 mL). The resultant composition was again allowed to settle. The pentane, and any residual heptane, was removed by evaporation using a rotary evaporator at 20° C. to produce GM3 containing encapsulated microparticles comprising gelatin and sodium fluorescein.

### Example B3—Encapsulated Hydrophilic Agent Microparticles

A pectin concentrate was prepared by dispersing pectin (20 g) CU025 (Herbstreith & Fox) in 90° C. water (380 mL) with moderate shear mixing, and heating the composition to 95° C. for 5 minutes. A solution of glucose (200 g) in water (165 g) with tartaric (4 g) acid was prepared and heated to 95° and pectin concentrate (90 g) added and the temperature maintained at 95° C. for 5 minutes. Next, a fluorescein dye (surrogate dye for hydrophilic therapeutic agent) was added when the temperature fell below 60° C., and the composition allowed to slowly cool for at least 12 hours to form a firm gel. The gel was then chopped into 1 cm cubes and cool to −80° C. to provide a rigid solid, and the cubes ground to a powder in a grinder, and returned to the refrigerator. Heptane was cooled to 80° C., and then added to the pectin powder with very high shear mixing, which further reduced the particle size. Membrane composition was added from Example A1 at 0.5% of the pectin particles, followed by an equal weight of 5% glycerol was added along with 0.4% tartaric acid in water with gentle mixing. This was allowed to settle, and then the supernatant heptane was poured off and any remaining heptane removed under vacuum in a rotary evaporator to provide a suspension of encapsulated microparticles comprising pectin and fluorescein.

### Example B4—Encapsulated Hydrophilic Agent Microparticles

Amidated pectin CU 025 4.5 g (Hammerstreith & Fox) in 85.5 g of water was dispersed and dissolved and heated to 95° C. and maintained for 5 minutes. Another solution of 200 g of glucose was dissolved into 165 g of water with heating to 95° C. The two solutions were combined, and then the solution was allowed to cool slowly and on reaching 60° C., 0.01% Sodium Fluorescein (surrogate dye for a hydrophilic biologically active substance) was added with gentle stirring. The resultant gel was set by standing overnight at −20° C. The pectin gel was then frozen to −80° C. to form a brittle solid which was ground to a fine powder while frozen. The powder was dispersed into 4× the volume of hexane at 10-80° C. and further ground to one micron particles with a colloid mill. Membrane composition from Example A1 was added to the particle suspension, then an equal volume of 1% tartaric acid and 20% xylitol in water was added to displace most of the hexane. The suspension of encapsulated microparticles in water was then put into a rotary evaporator to remove the remaining hexane to produce encapsulated microparticles comprising pectin and sodium fluorescein. To investigate encapsulation of the dye and release from the microparticles in a specific pH environment, the encapsulated microparticle suspension was added at 1% to pH 7.3 phosphate saline buffer at 37° C. and the encapsulated microparticles observed when the suspension was agitated on a slow bottle roll. Within 10 minutes, all encapsulated microparticles disintegrated, releasing the sodium fluorescein dye from the encapsulated microparticles.

### Example B5—Encapsulated Hydrophilic Agent Microparticles

Ultra Tex 4 pregelatinized starch (50 g) was dispersed in 310 g of water, and methylene blue dye (50 mg) added and slowly heated to 80° C. Next, the composition was cooled to 20° C. and allowed to gel for 2 hours, then chopped in heptane cooled to ^{˜}−80° C. The particle size was reduced to less than 2 mm with a blender, while maintained at ^{˜}−80° C. The particle size was then reduced to less than 2 microns with a high shear mixer. To the particles 3 ml of membrane composition extract from Example A1 was added then the composition was again subjected to shear. The composition was left for more than 8 hours at ^{˜}4° C. to allow for encapsulation. The upper layer of heptane was decanted and an equal volume of water added to the microparticles to form encapsulated microparticles comprising modified starch and methylene blue. Any remaining heptane floating on the surface was additionally removed.

### Example B6—Encapsulated Hydrophilic Agent Microparticles

Encapsulated microparticles were prepared comprising agar and methylene blue. Agar-agar (6 g) was dissolved in water (444 g), and methylene blue dye (30 mg) added and slowly heated to 95° C. Next, the composition was cooled to 20° C. and allowed to gel for 2 hours then chopped in heptane cooled to ^{˜}−80° C. The particle size was reduced to less than 2 mm with a blender, while maintained at ^{˜}−80° C. The particle size was then reduced to less than 2 microns with a high shear Silverson L5 mixer. To the particles were added 3 ml of isopropanol sheep's brain extract of Example A2, then again subjected to shear. The composition was then left for more than 8 hours at ^{˜}4° C. to allow for encapsulation. The upper layer of heptane was then decanted and an equal volume of water added to the microparticles to form encapsulated microparticles comprising agar and methylene blue. Any remaining heptane floating on the surface was additionally removed.

### Example B7—Encapsulated Hydrophilic Agent Microparticles

Encapsulated microparticles were prepared comprising pectin and a large molecule hydrophilic agent adalimumab (commercially available as Humira® from AbbVie Inc.). GM3 ganglioside encapsulated microparticles were prepared according to the procedure of Example B3 (except that adalimumab was used instead of fluorescein) to produce encapsulated microparticles.

### Example B8—Encapsulated Hydrophilic Agent Microparticles

Encapsulated microparticles were prepared comprising gelatin and a large molecule hydrophilic agent adalimumab. GM3 ganglioside encapsulated microparticles were prepared according to the procedure in Example B2 to produce encapsulated microparticles.

### Example B9—Encapsulated Hydrophilic Agent Microparticles

Encapsulated microparticles were prepared comprising agar and a large molecule hydrophilic agent adalimumab. GM3 ganglioside encapsulated microparticles were prepared according to the procedure in Example B6 (except that adalimumab was used instead of methylene blue) with the membrane composition of A2 to produce encapsulated microparticles.

### Example B10—Encapsulated Hydrophilic Agent Microparticles

Encapsulated microparticles were prepared comprising agar and a large molecule hydrophilic agent adalimumab. GM1 enriched encapsulated microparticles were prepared according to the procedure in Example B6 (except that adalimumab was used instead of methylene blue) to produce encapsulated microparticles.

### Example B11—Encapsulated Hydrophilic Agent Microparticles

Encapsulated microparticles were prepared comprising gelatin and a large molecule hydrophilic agent adalimumab. GM1 enriched encapsulated microparticles were prepared according to the procedure in Example B2 (except that adalimumab was used instead of sodium fluorescein) or B3 (except that gelatin was used in place of pectin) with the membrane composition of A2 to produce encapsulated microparticles comprising gelatin and adalimumab.

### Example B12—Encapsulated Hydrophilic Agent Microparticles

Encapsulated microparticles were prepared comprising pectin, starch and methylene blue. CU 025 Amide pectin Herbstreith & Fox, Germany (20 g) was added quickly to to hot water (380 g) with a high shear blender, then heated to 95° C. to hydrate the pectin producing a 5% pectin concentrate. Next, pre-gelatinised starch Ultra Tex 4, National Starch (10, 20 or 30 g) was added to tartic acid (2%) and methylene blue (0.01 g) in cold water (438, 428 or 418 g) with a magnetic stirring. The composition was heated to 95° C. then 90 g of the hot 5% pectin added with stirring. The composition was then allowed to cool and gel at 4° C. overnight. Next, 10 g of the composition was added to 200 mL of heptane and cooled to ^{˜}−70° C. in an ethanol/dry ice bath then chopped using a blender to less than 1 mm particles. The composition was then transferred to a tall cylinder and high sheer blending used to further reduce the particle size. Then a high shear Silverson mixer was used to reduce the particles size to ^{˜}1 micron. Next, 2 mL of isopropanol extract spray dried butter milk powder (as described in A1) was added and sheering continued and the composition allowed to equilibrate and microparticles to precipitate. Next, 0.5 mL of the precipitated microparticles was added to 3 mL of the tartic acid and gently mixed. The heptane was allowed to float to the surface and then removed.

### Example B13—Encapsulated Hydrophilic Agent Microparticles

Encapsulated microparticles were prepared comprising pregelatinized starch and Red Fluorescent protein. The encapsulated microparticles were enriched with GM3. Red Fluorescent protein (RFP which has a KDa ^{˜}27) 1 mg (BioVision) was centrifuged. The supernatant was removed and precipitate dissolved in 100 μL of water. UltraSperse pregelatinised starch (10%) was prepared by slowly adding 100 g to 900 g of cold water and leaving the gel overnight to equilibrate. 1.5 ml of 10% starch was collected a 3 ml syringe. The syringe plunger was removed and 254 of RFP added then mixed with a nearly matching spiral moving up and down until the colour was uniform. 200 ml of heptane was cooled to less than −70° C. in a blender, then with good manual agitation the starch/RFP was extruded slowly from the syringe allowing time for the thin extrusion to freeze. The blender was replaced by a Silverson L5 high shear mixer and the particle size further reduced, and then the resulting microparticles were encapsulated by a coating of 2 ml of buttermilk powder extract as described in A1. The microparticles were allowed to settle and the supernatant removed and replaced by cold (^{˜}−70° C.) pentane. The microparticles were again allowed to settle then 40 ml of microparticles in pentane recovered. 10% glucose solution was prepared and 1.5 ml taken in a test tube. 0.2 ml of extract was added giving a noticeable milkiness in the solution to indicate saturation. 0.25 ml of concentrated microparticles were slowly added above the glucose solution and the microparticles penetrated into the surface of the solution, providing a nicely dispersed suspension of bright pink microparticles.

### Example B14—Encapsulated Hydrophilic Agent Microparticles

Encapsulated microparticles comprising 1% agar were prepared by adding 0.8 mL of liquid agar (1.25 mg) to a 250 mL beaker and 198 g MQ water at a temperature of 95° C. or above. The solution was then heated on a hotplate with a stirrer up to or above 97° C. for at least 10 mins. After heating the weight was checked and made back up to 200 g with MQ water. The solution (1.5 mL) was then drawn into 3 mL syringes, capped and placed in a 45° C. water bath for at least 30 mins. After incubation the plunger was removed from the syringes and 10 μL of MQ water or 104 (controls) of red fluoresecent protein (R-PE) added to the agar which was mixed with a small spatula. The plungers were replaced and let to set at room temperature for 1 hr before storing at 4° C. at least overnight.

In a −70° C. alcohol bath (methylated spirits and dry ice) the gels were extruded from the syringe into a plastic vessel containing 200 mL of −40° C. or below heptane and mixed by a hand held blender (bamix) for 1 min, followed by shearing with a Silverson mixer for 30 secs at high speed. Dropwise 1 mL of extract from Example A1 was added whilst the Silverson was running and sheared for a further 30 seconds. The preparation was removed from the alcohol bath and poured into a plastic beaker and the particles left to settle for approximately 2 min. Excess heptane was removed by pouring, leaving a small amount at the bottom of the beaker containing the microparticles that can be poured into a 50 mL falcon tube. The preparation was then stored at 4° C. In some instances the microparticles were subjected to washing twice in 2× volume of pentane letting the mixture settle for 1 min each time before pouring off the pentane. Then, 10 mL of fresh pentane was added to the 50 mL tube and the preparation stored at 4° C.

### Example B15—Encapsulated Hydrophilic Agent Microparticles

15% pre-gelatinised starch microparticles were prepared by adding 0.8 mL of pre-gelatinized starch 25 μL (or 0.225 g of pregelatinized starch) to a 3 ml syringes with 1.275 g of ice cold MQ water and mixing quickly with a small spatula followed by mixing with a spiral stirrer until homogeneous. The mixture was then left at room temperature for at least 1 hr to gelatinise. After incubation the plunger was removed from the syringes and 10 μL of MQ water or 10 μL (controls) of R-PE red fluoresecent protein added to the starch which was mixed with a small spatula. The plungers were replaced and let to set at room temperature for 1 hr before storing at 4° C. at least overnight.

In a −70° C. alcohol bath (methylated spirits and dry ice) the gels were extruded from the syringes into a plastic vessel containing 200 mL of −40° C. or below heptane and mixed by a hand held blender (Bamix) for 1 min, followed by shearing with a Silverson mixer for 30 secs at high speed. Dropwise 1 mL of extract from Example A1 was added whilst the Silverson was running and sheared for a further 30 seconds. The preparation was removed from the alocohol bath and poured into a plastic beaker and the particles left to settle for approximately 2 min. Excess heptane was removed by pouring, leaving a small amount at the bottom of the beaker containing the microparticles which were then poured into a 50 mL falcon tube and stored at 4° C. In some instances the microparticles were subjected to washing twice in 2× volume of pentane letting the mixture settle for 1 min each time before pouring off the pentane. Then, 10 mL of fresh pentane was added to the 50 mL tube and the preparation stored at 4° C.

### ii. Encapsulation and Loading Efficiency Studies

### Example B16—Encapsulated Hydrophilic Agent Microparticles

Encapsulated microparticles were prepared comprising pectin and a large molecule hydrophilic agent adalimumab of Example B7. The amount of adalimumab encapsulated within the microparticles was also measured. The methodology involved: (1) adding a 50 ul of TRIS HCl pH 9.0 to 500 ul of GM formulation then adding 450 ul of TRIS HCl pH 7.51 to quantify the adalimumab in lysed microparticles and the supernatant; and (2) separating the supernatant from the microparticles by filtering 1 ml of formulation through a 0.2 um syringe filter to quantify the adalimumab in the outside the microparticles in the suspending fluid. Adalimumab was quantified using an Elisa kit as set out in the draft report. The encapsulation efficiency was then be calculated as follows: (lysed−supernatant)/lysed. The encapsulation efficiency percentage of adalimumab that was encapsulated for this formulation was shown to be above 90%. This provides a surprisingly advantageous loading efficiency of the procedure to encapsulate a large molecule hydrophilic agent of adalimumab.

### Example B17—Encapsulated Hydrophilic Agent Microparticles

Encapsulated microparticles were prepared comprising pectin, gelatin, or agar, and the large molecule hydrophilic agent adalimumab. The encapsulated microparticles were enriched with the ganglioside GM1 or GM3. Encapsulated microparticles of the following formulations were prepared: GM3/pectin/Adalimumab (Example B7), GM3/gelatin/Adalimumab (Example B8), GM3/agar/Adalimumab (Example B9), GM1/agar/Adalimumab (Example B10) and GM1/gelatin/Adalimumab (Example B11). The amount of adalimumab encapsulated within the microparticles was also measured as described above for B16. The B9 and B11 formulations were tested within the limits of detection using the Elisa kit.

### Example B18—Encapsulated Hydrophilic Agent Microparticles

This study was conducted to measure the amount of R-phycoerythrin red fluorescent protein (R-PE) that is encapsulated within agar or starch microparticles. Encapsulated microparticles were prepared comprising agar or starch as described above in B14 and B15. The encapsulated microparticles were enriched with the ganglioside GM3. Four GM3 microparticle formulations were prepared comprising either agar or starch resuspended in heptane or agar and starch resuspended in pentane (B14 and B15)

On the day of the encapsulation study approximately 100 μg of the microparticles were removed from the tube with a spatula and placed into a 2 mL tube and the weight recorded. 500 μL of sterile 10% glucose+extract (20 mL 10% Glucose+200 μL of extract) was added on top of the microparticles. The tube was then placed in a small flask and connected to the rotary evaporator and the heptane evaporated for 10 min. The tube was then weighed again and the weight recorded before it was centrifuged at 2000×g for 30 sec and the supernatant removed.

50 μl of the supernatant was set aside while the remaining amount was filtered through a 0.22 um syringe filter and the tube containing the remaining microparticle pellet weighed. This left three fractions: supernatant, filtered supernatant and microparticle pellet. Fluorescence was measured on the FlexStation II apparatus in a clear bottom, black walled 96 well plate. 50 μl of each of the three fractions were added to individual wells and the readings taken using an excitation wavelength of 566 nm and an emission wavelength of 575 nm. Results are shown in Table 1.

TABLE 1
Retention of red fluorescent protein in GM3 1% agar or 15%
starch microparticles prepared in heptane or heptane with
additional pentane wash steps.

| | | 2-Heptane/ | | 4-Pentane/ |
|---|---|---|---|---|
| | 1-Heptane/agar | starch | 3-Pentane/agar | starch |
| % Retention | 44.11 | 41.72 | 25.55 | 38.88 |

### Example B19—Encapsulated Hydrophilic Agent Microparticles

This study was conducted to determine whether increasing the amount of extract added during the manufacturing process improves the encapsulation and retention of R-phycoerythrin red fluorescent protein (R-PE) within agar microparticles. Microparticles were prepared as described in B14 except that the concentration of ganglioside extract added from Example 1A added was increased from 1 mL to 3 mL. On the day of the encapsulation study approximately 2×100 μg of the microparticles were removed from the bottom of the tube with a spatula and weighed into 2 mL tubes and the weight recorded. 500 μl of sterile 10% glucose+extract (20 mL 10% Glucose+2004 of extract) was added on top of both tubes of microparticles and one sample had an extra 10 μl of extract added to it. Individually the tubes were placed in a small flask and connected to the rotary evaporator and the heptane evaporated for 10 min. Each tube was then weighed again and the weight recorded before they were centrifuged at 2000×g for 30 sec and the supernatant removed. 50 μl of the supernatant was set aside while the remaining amount was filtered through a 0.22 um syringe filter and the tubes containing the remaining microparticle pellets weighed. This left three fractions: supernatant, filtered supernatant and microparticle pellet for each preparation.

50 μl of each fraction was loaded into a black walled clear bottomed 96 well plate. The remaining microparticles were combined and 100 μl of 10% glucose+extract added to attempt to wash any remaining R-PE out of the supernatant. The tube was centrifuged at 200×g for 30 seconds and 50 μl of just the supernatant and microparticle pellet were loaded onto the 96 well plate. 50 μl of 10% glucose+extract was also loaded into a well as a negative control. The plate was read in the FlexStation II, Excitation 566/Emission 575. Results are shown in Table 2.

TABLE 2
Retention of red fluorescent protein in GM3 1% agar microparticles.

| | Agar/R-PE + 10 ul | | Agar/R-PE washed |
|---|---|---|---|
| | extract | Agar/R-PE | microparticles |
| % Retention | 100.23 | 99.59 | 100.00 |

### Example B20—Encapsulated Hydrophilic Agent Microparticles

This study was conducted to test the retention efficiency of the 1% agar GM3 microparticles containing mCherry. Microparticles were prepared as described in B14 except that the after incubation in a 45° C. water bath 5, 10 or 20 ug of mCherry was added to the agar in place of 10 μL of MQ water or 10 μL of red fluoresecent protein. Pentane washes were not used in the preparation of these microparticles.

On the day of the study approximately 200 μg of the microparticles were removed from the bottom of the tube with a spatula and placed into 2 mL tubes and the weight recorded. 800 μl of sterile 10% glucose+extract (10 mL 10% Glucose+50 μl of extract) was added on top of the tubes of microparticles. Individually the tubes were placed in a small flask and connected to the rotary evaporator and the heptane evaporated for 10 min. Each tube was then weighed again and the weight recorded before they were centrifuged at 2000×g for 30 sec and the supernatant removed. 50 μl of the supernatant was set aside while the remaining amount was filtered through a 0.22 um syringe filter and the tubes containing the remaining microparticle pellets weighed. This left three fractions: supernatant, filtered supernatant and microparticle pellet for each preparation. To a clear bottomed black walled 96 well plate 50 μl of the supernatant, filtered supernatant or microparticle pellet was added in triplicate. The plate was then read using the FlexStation II, Excitation 587/Emission 610. Results are shown in Table 3.

TABLE 3
Retention of mcherry within GM3 1% agar microparticles.

| | | | Agar/mCherry |
|---|---|---|---|
| | Agar/mCherry 5 ug | Agar/mCherry 10 ug | 20 ug |
| % Retention | 87.77 | 85.32 | 93.68 |

### Example 821-Encapsulated Agent Microparticle Formulation Study

A further formulation study was undertaken to identify encapsulated microparticles incorporating a range of therapeutic agents and fluorophores. The therapeutic agents and fluorophores investigated were selected from trastuzumab (Herceptin®), peginterferon alfa-2a (Pegasys®), adalimumab (Humira®), leuprolide (Eligard®), R-phycoerythrin (R-PE), and Methylene blue (MB).

Preparation of formulations A series of studies were performed on microparticle formulations coated with lipid (ganglioside) extracts. Two formulation processes were used. For the first process involving a solidified gel core, a first step was the preparation of a solid gel such as 15% w/v starch in water into which the therapeutic/fluorophore was dispersed. The therapeutic/fluorophore in the solid gel was then slowly extruded into ice cold heptane while mixing with a kitchen hand blender to break the starch into smaller pieces. The gel particles were further sheared in a Silverson L4RT mixer. A lipid (ganglioside) extract (i.e. membrane composition) was added during the Silverson mixing step to encapsulate (e.g. coat) the particles with surface lipids including gangliosides. Different crops of encapsulated particles were collected based on how fast the particles settled in heptane. The collected particles were dispersed in a solution of glucose solution, with or without added lipid (ganglioside) extract, for analyses. The particles can also be prepared under nitrogen to reduce or prevent water adsorption due to exposure to air.

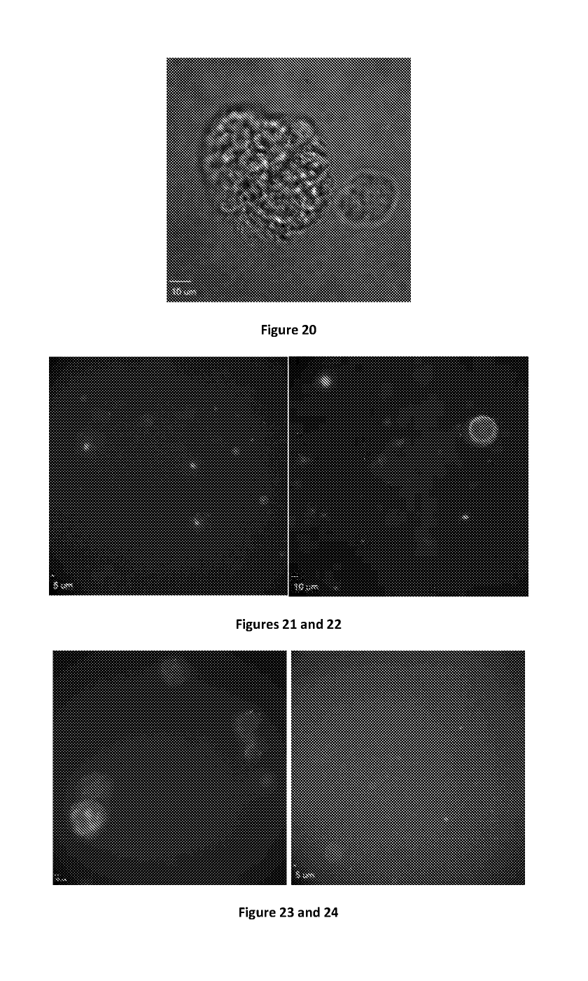

Variations in the above process can result in the formation of more or less agglomerated particles and sizes of agglomerated particles. The encapsulated particles, once prepared, were introduced into a glucose solution for use in further studies. Small micron sized encapsulated particles were identified in the prepared formulations, and to varying amounts larger agglomerated particles were also present (see FIG. 20). Variations of the following process features can provide differences in the degree of agglomerated encapsulated particles present in the formulation relative to individual encapsulated particles: i) starch type and concentration in the gel (e.g. 15 wt % produces an adequate gel), ii) Silverson mixing time and speed, iii) volume and type of ganglioside extracts added during particle preparation in heptane and during dispersion in the glucose solution, iv) settling time of the particles after mixing and v) the collection of different crops +/−washing of settled particles.

For a second process involving a liquid core, the first step was the preparation of a liquid 2% starch gel into which the therapeutic protein was dispersed. The gel was slowly added into ethyl acetate containing the lipid (ganglioside) extract and mixed (e.g. with the Silverson L4RT mixer). Different crops of particles were collected based on how fast the particles settled in ethyl acetate. The collected particles were finally dispersed in a solution of glucose with or without added lipid (ganglioside) extract. In initial trials the particles were prepared with mixing during starch addition to the ethyl acetate provided by a magnetic stirring bar, bath sonicator, benchtop ultrasonicator or ultrasonic probe. The particles produced on mixing with these methods were relatively large in size (>20 micron). It appeared that the size of the particles formed and size of agglomerated particles was dependent on the mixing/shear provided at the time that the starch entered the ethyl acetate and smaller particles could result if mixing was continued at that time. For this reason the Silverson mixer was used as a means of providing mixing during starch addition to the ethyl acetate. Micron sized particles formed on mixing with the Silverson. Variations of the following process features can provide differences in the degree of agglomerated encapsulated particles present in the formulation: i) gel concentration (2-5% worked well), ii) mixer type and mixing time, iii) volume and type of lipid extracts used during particle preparation and during dispersion in the glucose solution, iv) settling time and v) the collection and washing of different crops of settled particles.

For reduction in conglomeration of particles, smaller volumes of lipid extract can be added and isopropanol can also be added to the heptane to increase the solubility of the lipids in the solvent and reduce lipid accumulation around the starch particles. To further reduce agglomeration the ganglioside extracts can also be filtered prior to use to remove particulates.

The encapsulated agent preparations involve therapeutic/fluorophore loaded particles coated with a ganglioside containing lipid extract. The gangliosides facilitate active uptake of the therapeutic/fluorophore loaded particles across the intestinal epithelium. Different types of colloidal systems, aside from starch based microparticles, can also be coated with gangliosides. These systems could be selected to be digestion resistant particles.

### C. In Vitro Studies of Endocytosis of Encapsulated Microparticles

### Example C1—Encapsulated Hydrophobic Agent Microparticles

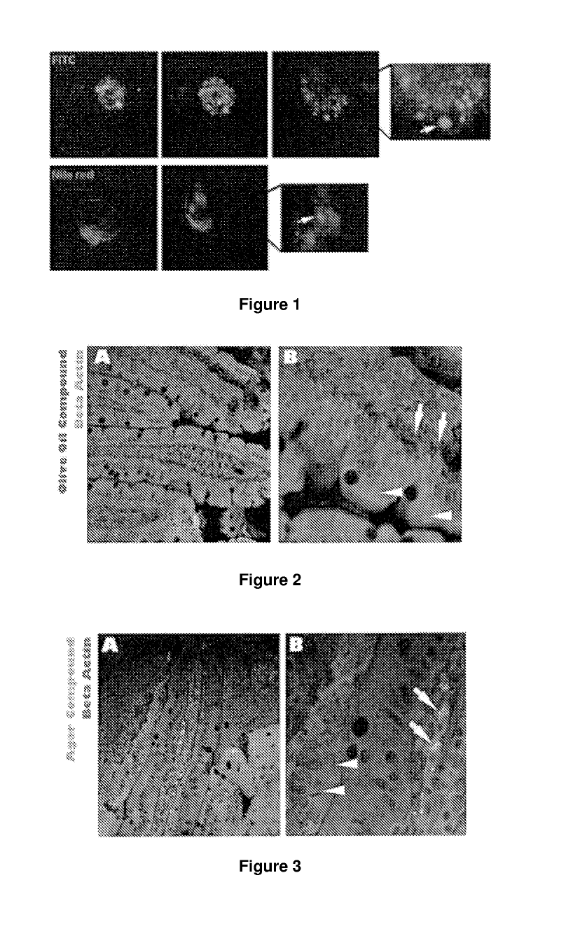

A composition was prepared following the same procedure as described for Example B1. Caco-2 cells were used to examine endocytosis of the encapsulated microparticles into the cell. These cells (passage 4), were cultured using standard media (Gotz et al., 2010), in 200 microliters of Roswell Park Memorial Institute (RPMI) medium containing 10% Foetal Bovine Serum (FBS) and 1% Penicillin/Streptomycin for a week following thawing, on 8 well chamber slides. They were then treated with differing concentrations of the encapsulated microparticles (in a range of about a 1:10 to a 1:1000 dilution, for example about 1:100) for 18 hours, and then processed for immunocytochemistry and imaged on a confocal microscope (FIG. 1).

### Example C2—Encapsulated Hydrophilic Agent Microparticles

A composition was prepared following the same procedure as described for Example B2, except the gelatin was replaced with agar powder (4 g) to produce a encapsulated microparticle comprising agar and sodium fluorescein. Caco-2 cells were used to examine endocytosis of the compounds into the cell. These cells (passage 4), were cultured using standard media (Gotz et al., 2010), in 200 microliters of RPMI containing 10% Foetal Bovine Serum (FBS) and 1% Penicillin/Streptomycin for a week following thawing, on 8 well chamber slides. They were then treated with differing concentrations of the compounds (in a range of about a 1:10 to a 1:1000 dilution, for example about 1:100) for 18 hours, and then processed for immunocytochemistry and imaged on a confocal microscope (see also FIG. 1).

### Example C3—Encapsulated Hydrophilic Agent Microparticles

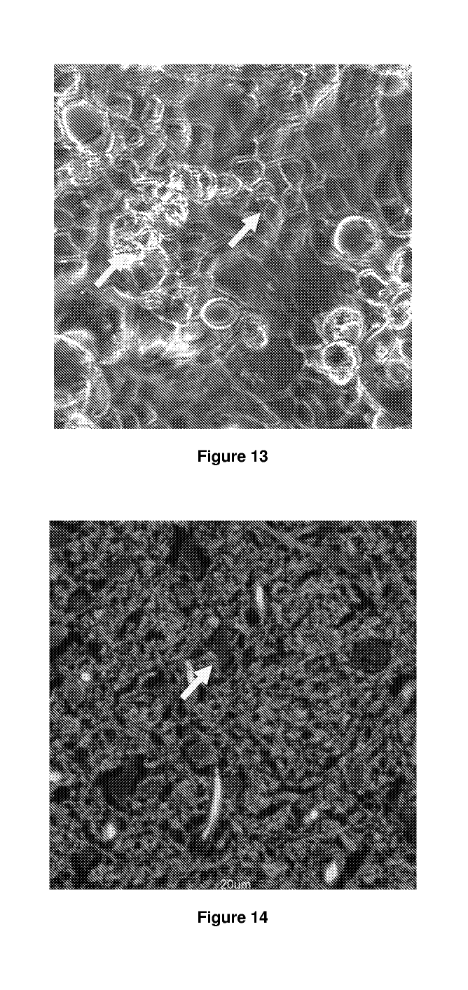

A composition was prepared following the same procedure as described for Example B8. The encapsulated adalimumab microparticles were also stained with methylene blue. Caco-2 cells were used to examine endocytosis of the agents into the cell. These cells (passage 4), were cultured using standard media (Gotz et al., 2010), in 200 microliters of RPMI containing 10% Foetal Bovine Serum (FBS) and 1% Penicillin/Streptomycin for a week following thawing, on 8 well chamber slides processed for immunocytochemistry and imaged on a confocal microscope. FIG. 13 shows microparticles of Example C3 accumulating in cells (see arrow).

### Example C4—Encapsulated Hydrophilic Agent Microparticles

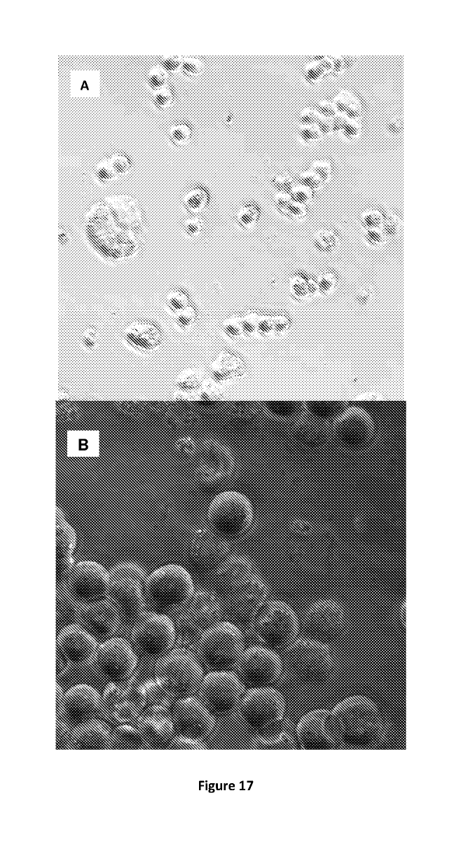

Compositions comprising GM1 and GM3 microparticles comprising agar, adalimumab and methylene blue were prepared as described in example B9 and B10 except that both adalimumab and methylene blue were encapsulated in the microparticles. HT-29 cells were spiked with 10 μL of formulation for 15 min. Supernatant was removed and the cells washed twice with PBS. Endocytosis of microparticles was then assessed with confocal microscopy. A representative image of microparticle endocytosis in HT-29 cells is shown in FIG. 17B compared to controls in FIG. 17A.

### Example C5—Encapsulated Hydrophilic Agent Microparticle

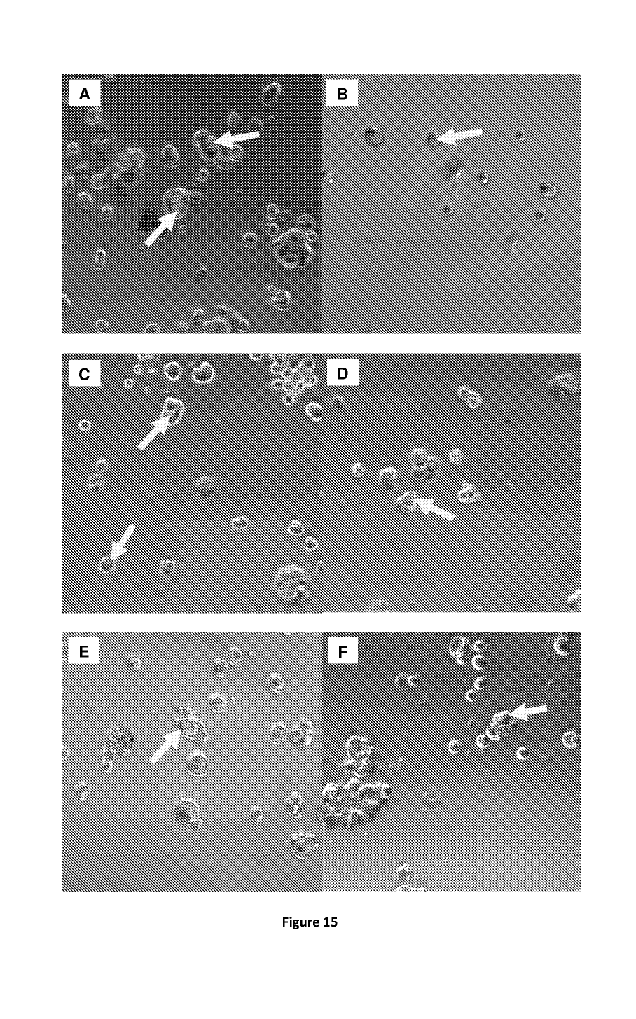

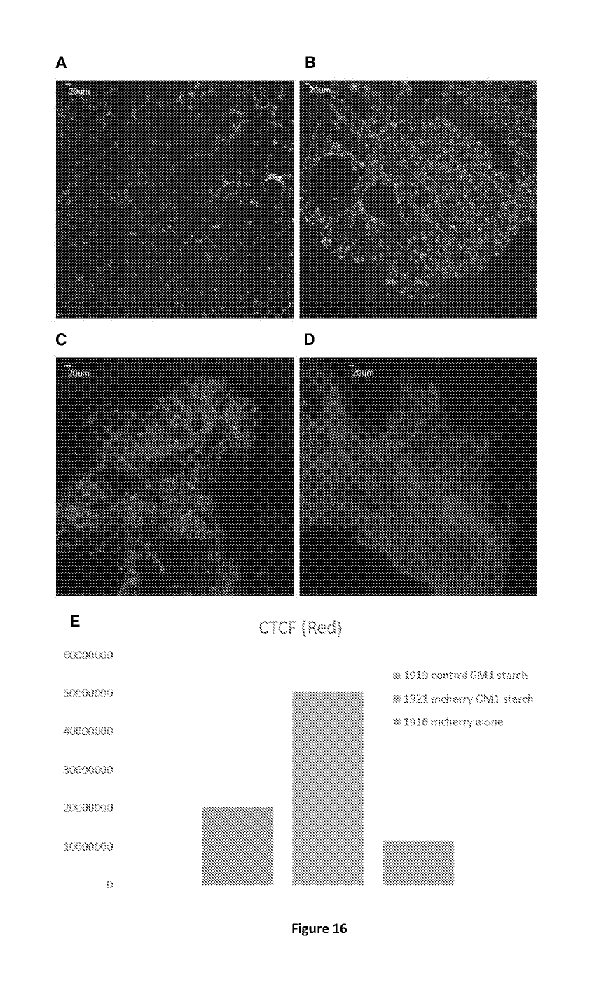

Microparticles of agar and methylene blue in heptane were prepared as previously described. A solution of soy bean lecithin in isopropanol with similar solids concentration to a butter milk powder isopropanol extract was also prepared. Mixtures of 100%, 50%, 20%, 10%, 5% and 1% of extract to lecithin were prepared (referred to as 100 dose, 20 dose, 10 dose, 5 dose and 1 dose, respectively). Aliquots (5 mL) of the microparticle were added to 10 mL screw top vials. Next, 100 μL of each of the mixtures were added to the microparticle aliquots and held overnight at 4° C. Water (4 mL) was added to each aliquot and excess heptane was removed. Caco-2 cells were grown in a cell culture plate and 200 μL of microparticle preparations added to the Caco-2 cells and incubated at 37° C. for 30 mins. Sodium azide 0.2% was added to limit further endocytosis was then added and rinsed once with PBS. FIG. 15 shows that microparticles were endocytosed into Caco-2 cells at all of the doses tested. Cells were then examined for endocytosis using confocal microscopy. The FIG. 15 images demonstrate that at least a 1 dose ganglioside extract is sufficient to trigger endocytosis in vitro in Caco-2 cells.

### D. In Vivo Results of Transcytosis and Endocytosis of Encapsulated Microparticles

### i. Studies of Transcytosis into the Lymphatic System

### Example D1—Encapsulated Hydrophobic Agent Microparticles

A composition was prepared following the same procedure as described for Example B1. An in vivo study was carried out in rats and tissue sections were processed, imaged and analysed. Histological sections from animals treated by intragastric administration of the encapsulated microparticles comprising Nile Red were processed by immunohistochemistry and imaged using confocal microscopy.

### Example D2—Encapsulated Hydrophilic Agent Microparticles

A composition was prepared following the same procedure as described for Example B2 except the gelatin was replaced with agar powder (4 g) to produce a encapsulated microparticle in a hydrophilic carrier comprising agar and sodium fluorescein. An in vivo study was carried out in rats and tissue sections were processed, imaged and analysed. Histological sections from animals treated by intragastric administration of the encapsulated microparticles comprising fluorescein were processed by immunohistochemistry and imaged using confocal microscopy.

### Procedure and Results for Examples D1 and D2

Histology

The duodenum, jejunum and ileum regions of the small intestine were treated and then dissected in the following manner:
irrigate/flush the small intestine with saline to remove undigested contents of the lumen
dissect 2 samples each being 1.5 cm long from each of the 3 segments of intestine (i.e., duodenum, ileum and jejunum)
fix tissues by immersing in 4% formalin in buffered saline, made fresh from paraformaldehyde
perform paraffin embedding of 1 sample from each of the 3 segments
cut approximately 100 serial sections 10 urn thick

place on “Superfrost+” glass slides, 3 serial sections per slide, and slides numbered consecutively in the series.

Immunohistochemistry and Confocal Microscopy Immuno-histochemistry was done according to a standard protocol as previously described in Azari et al., (2006) J Neuropathol Exp Neurol 65:914-929. Sections were deparaffinised and washed. Non-specific binding was blocked using skim milk. Sections were washed and treated with blocking buffer containing the primary monoclonal anti-beta actin antibody to stain the intestinal cell cytoskeletons (1:200 dilution) at room temperature on a rocker. Following washing with phosphate buffered saline (PBS), the sections were exposed to a fluorescent secondary antibody. Two separate secondary antibodies were used according to the fluorophores used in the compounds. Therefore, the intestinal samples administered the 488 nm fluorescently (green) labelled compound in agar (Example D1), consisted of a secondary anti-mouse 555 nm (red) antibody to detect the anti-beta actin monoclonal antibody. However, the intestinal samples administered the 555 nm Nile red (red) labelled compound in olive oil (Example D2) consisted of a secondary anti-mouse 488 nm (green) antibody to detect the anti-beta actin monoclonal antibody. The secondary antibody step was for 2 hours at room temperature, followed by PBS washes and a 30 minute 4′,6-diamidino-2-phenylindole (DAPI) nuclear counterstain. This was followed by washes and cover slipping using fluorescent mounting medium.

The sections were then imaged using a Leica TCS NT upright confocal microscope. The images were processed using Adobe Photoshop CS5 software.

### Results

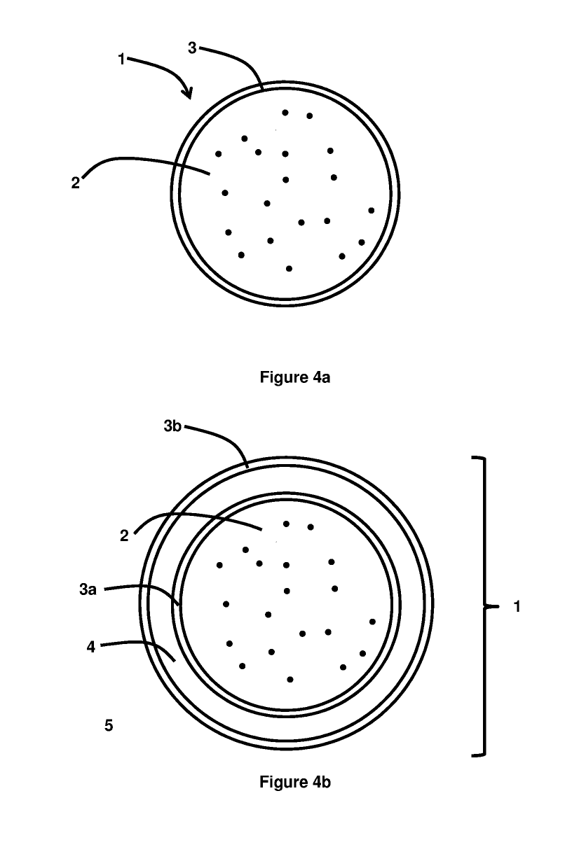

All samples resulted in the identification of beta actin immunopositive intestinal cells from the mucosa right through to the lamina propria and muscularis mucosa of the wall of the small intestine at all three anatomical regions (proximal to distal). Similarly, all fluorophores within the compounds administered to the rat small intestine showed marked uptake through the mucosa with evenly sized fluorescent vesicles (Arrows) demonstrated intracellularly from the mucosal apical surface, transferred through to the basal surface. Furthermore, a collection of these vesicles appeared within the lamina propria, which by this stage were large coalesced particles and could only be observed within the lacteals and subsequent lymphatic drainage ducts (arrows in FIG. 2) of the small intestine. This was consistent for all samples, which seemed to be best taken up by the jejunum with greater intensity when imaging the olive oil compounds but the agar compound was taken up with greater intensity by the ilium (FIG. 3).

### Conclusions

The encapsulated microparticles of Examples D1 and D2 were able to enter the mucosal cells of the intestine and cross into collecting lymphatic vessels. While the encapsulated microparticles of Example D1 was taken up with greater intensity by the jejunum, the encapsulated microparticle comprising agar of Example D2 was taken up more intensely by the ilium.

### ii. Studies of Transcytosis Through Lymphatic System into Adipose Tissue

### Example D3—Encapsulated Hydrophilic Agent Microparticles

The delivery into adipose tissue of rats was demonstrated using encapsulated microparticles of Example B5.

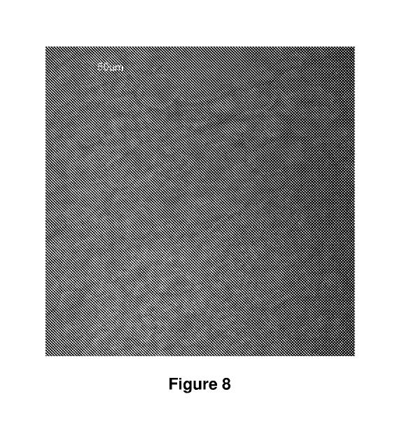

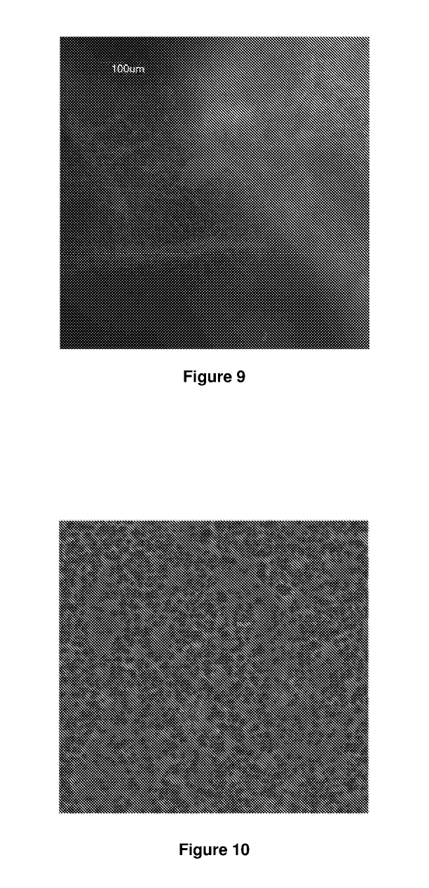

The microparticle suspension (1.5 mL) was administered to a ^{˜}300 g rat. After 4 hours the rat was sacrificed and dissected to obtain brown fat from the deposit on the back. Sections of the brown fat were examined using confocal microscopy. Accumulation of the methylene blue was detected in the adipose tissue (see FIG. 8 and FIG. 9) indicating that microparticles transverse the lymphatic and cardiovascular system intact and are successfully delivered to adipose tissue.

As seen in FIG. 8, the adipose cells appear as ellipsoid structures with the boundaries enriched in blue (about 2×1 microns thick). The microparticles were transcytosed through the intestine and delivered to the adipose cells. The thickness of the blue boundaries support that the microparticles were absorbed by endocytosis. The image in FIG. 8 supports that GM3 rich membrane composition encapsulating the microparticles provides targeted delivery to brown fat and endocytosis into the cells. As seen in FIG. 9, the microparticles transcytosed through the intestine and were delivered to the adipose cells. The image supports that the methylene blue stained microparticles were efficiently delivered to the brown fat and the delivery was targeted by the GM3 membrane coating.

### iii. Studies of Oral Administration and Transcytosis to Adipose Tissue and Lymphatic System

### Example D4—Encapsulated Hydrophobic Agent Microparticles

Studies were performed to confirm that encapsulated microparticles can be delivered intact through the gastrointestinal tract. Encapsulated microparticles were prepared according to Example B1.

### Example D5—Encapsulated Hydrophilic Agent Microparticles

A composition of Example D5 was prepared following the same procedure as described for Example B3 except that India Ink was used in addition to sodium fluorescein to produce an encapsulated microparticle comprising pectin, India ink and sodium fluorescein and a hydrophilic carrier.

### Procedure and Results for Examples D4

In this study lipid-soluble Red Nile dye (intended as a surrogate for lipid-soluble therapeutic agents) was encapsulated. The D4 formulation was administered by oral gavage to rats, and the absorption and distribution of the microparticles within the rats was then examined using confocal microscopy.

Animals

Sprague-Dawley rats (14 males), age range at start 10 to 16 weeks were kept in a controlled environment (targeted ranges: temperature 21±3° C., humidity: 30-70%, 10-15 air changes per hour, under barrier (quarantine)conditions). Temperature and humidity were continuously monitored. Light conditions varied from 30 Lux to 400 Lux depending on cage position. A standard rodent diet (Barastoc) and tap water were provided to the animals ad libitum and given fresh food and water as required. Cages were changed twice a week. Animals were labelled by ear tag.

Formulation Administration

Formulations as described for Example D4 were administered by oral gavage to the rates and the absorption and distribution of the microparticles within the rats was then examined using confocal microscopy. Administration was performed at a dosing volume of 2.5 ml per rat for liquid gavage, or in capsules at an equivalent dose. The formulations were administered daily by oral gavage at 2 pm. This study was a single dose study.

Animal Observations

Animals were sacrificed by carbon dioxide asphyxiation at prescribed times after administration of the formulations. Mesenteric tissue was quickly excised and spread on a 10 ml petri dish sitting on a block of ice. A section of tissue from the proximal gastrointestinal tract was excised and placed onto specific plates for confocal microscopy.

Group 1: Two rats were intragastrically gavaged with 2.5 mL of microparticles containing Nile red dye as a surrogate for lipophilic drugs (Example D4). After gavage (60 minutes), the brown fat pad from the suprascapular region of the rats was removed and examined by confocal microscopy. As shown in FIG. 10, there was clear evidence of accumulation of Nile red dye in brown fat. This indicates that the microparticles traversed the intestinal villi, the lymphatic system, and the cardiovascular system intact, and were disrupted upon reaching the brown adipose tissue.

Group 2, Group 3 and Group 4: Two rats in a fed state were given 2.5 mL liquid microparticle formulations containing fluorescein by intragastric gavage. One rat was killed 30 min after the gavage, and the other after 60 min. The mesentery was dissected out and spread on an ice block for confocal microscopy. One rat was fasted overnight and then given 2.5 mL liquid microparticle formulations containing fluorescein and Nile red dye by intragastric gavage. The rat was killed 20 min after the gavage. The mesentery was dissected out and spread on an ice block for confocal microscopy. One rat was fasted overnight and then given 2.5 mL liquid microparticle formulation containing fluorescein and Nile red dye by intragastric gavage. The rat was killed 60 min after the gavage. The mesentery was dissected out and spread on an ice block for confocal microscopy. In each case (Groups 2-4), significant autofluorescence was observed, precluding observation of movement of the formulations. Further optimization of appropriate dyes was undertaken to reduce and finally eliminate autofluorescence.

### Analyses and Conclusions

The study was designed to assess the uptake of the microparticles of Example D4 in the mesenteric lymphatic system. The results showed that the Example D4 formulation was delivered through the intestinal villi and into the lymphatic system intact, which demonstrates that the dye was delivered without being disrupted inside the intestinal lumen or the villi. In this study, a large concentration of the microparticles (containing Nile red dye) was found to have opened up within the brown adipose tissue, releasing the Nile red dye.

Encapsulated microparticles of dyed oil (Nile red dye) were orally administered to rats. A large concentration of the microparticles was found to have reached the brown adipose tissue intact and then was degraded to release the Nile red dye (FIG. 10). This indicated that the microparticles traversed the lymphatic circulatory system and the cardiovascular circulatory system intact. The Nile red dye was intended to act as a surrogate for lipophilic drugs. FIG. 10 shows that some of the microparticles opened up within the Peyer's patches, an effect that increased over time.

### iv. Studies of Oral Administration and Transcytosis to the Blood Brain Barrier

### Example D6—Encapsulated Hydrophilic Agent Microparticles

Studies were performed to confirm that encapsulated microparticles can be delivered intact across the blood brain barrier. Encapsulated microparticles were prepared according to Example B6.

### Example D7—Encapsulated Hydrophilic Agent Microparticles

Studies were performed to confirm that encapsulated microparticles can be delivered intact across the blood brain barrier. Encapsulated microparticles were prepared according to Example B2, except that methylene blue was used instead of sodium fluorescein.

### Procedure and Results for Examples D6 and D7

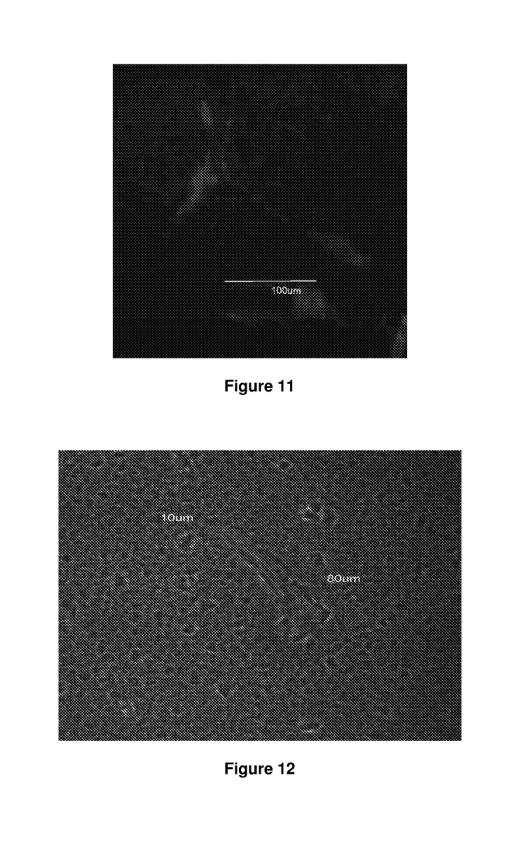

In the first study, for example D6, water soluble methylene blue (intended as a surrogate for water-soluble biologically agents) and the large molecule agar were included within novel encapsulated microparticles. In the second study, for Example D7, water soluble methylene blue (intended as a surrogate for water-soluble biologically agents) and gelatin were included within novel encapsulated microparticles. Both formulations were administered by oral gavage to rats after overnight fasting. Rats were returned to their cages with food, and then one rat sacrificed at 2 hours, one at 4 hours, and one at 7 hours. Each of the three rat brains were dissected on to confocal dishes, and confocal microscopy was performed. The 4 hour timepoint produced the greatest presence of intact microparticles containing methylene blue, which accumulated in the brain parenchyma (on the brain side of the blood brain barrier see FIG. 11 and FIG. 12). The 2 hour and 7 hour samples also demonstrated accumulation of intact microparticles, arguably with more microparticles observed at the 2 hour point than the 7 hour point. As the tissue dissected was the brain, this study clearly shows the methylene blue was on the brain side of the blood brain barrier. There was also evidence of accumulation within neural cells. The spatial set-up of the brain appeared to affect the visibility of microparticles under confocal microscopy; at the 7 hour time point, not much methylene blue was visible from the top down, but then the brain position was inverted and a great deal more methylene blue was visible from the bottom up. These images demonstrate that encapsulated microparticles comprising a large molecule (agar) and methylene blue traversed the intestinal villi through transcytosis (the “primary” transcytosis through the intestinal epithelium), the lymphatic system, and the cardiovascular system intact, and then traversed the blood brain barrier intact (the “secondary” transcytosis through the blood brain barrier). This finding is surprisingly advantageous as delivery through the blood brain barrier of large molecules by oral administration is rarely reported in the literature.

In the second study, the same protocol as above was applied to two rats, with one being gavaged with the encapsulated particles of Example D6 and the other with Example D7. Both rats were sacrificed at 4 hours, their brains were dissected, placed on confocal dishes, and confocal microscopy was performed.

The microparticles of both Example D6 and Example D7 and methylene blue were observed in the brain. In addition to the confocal microscopy, one brain hemisphere from each rat was analysed by paraffin processing, cutting and hematoxylin and eosin staining of 150 sections per brain. Slides from the agar formulation, rather than the gelatin formulation (Example D6), were then stained using a silver-based stain that would not interfere with methylene blue fluorescence. Confocal microscopy was then performed on the stained slides, and the images provided clear support that the encapsulated microparticles of Example D6 remained intact within neural cells (see arrow in FIG. 14). This indicates, as above, that there was primary transcytosis across the intestinal barrier, secondary transcytosis across the blood brain barrier, and then tertiary endocytosis into neural cells for a large molecule.

### v. Studies of Oral Administration and Transcytosis to Adipose Tissue

### Example D8—Encapsulated Hydrophilic Agent Microparticles

To confirm that encapsulated microparticles can be delivered to adipose tissue. GM3 or GM1 1% agar and 15% starch microparticle formulations containing fluorescent mCherry (29 kDa fluorescent protein) were prepared. Microparticle formulations tested in this study were as follows: 1. Agar—Control—GM1; 2. Agar—Control—GM3; 3. Agar—mCherry —GM1; 4. Agar—mCherry—GM3; 5. Starch—Control—GM1; 6. Starch—Control—GM3; 7. Starch—mCherry—GM1; 8. Starch—mCherry—GM3.

GM3 microparticles were prepared as described in B14 and B15 except that 100 μg of mCherry or 100 μL of MQ water was added in place of 10 μL of red fluoresecent protein or 10 μL of MQ water, and 6 mL of the extract from example A1 was added during blending. The microparticles were stored in heptane (pentane washes were not used during the preparation of these microparticles). GM1 microparticle formulations were prepared as described above for GM3 microparticles except that the extract from Example 1A was replaced with the extract from Example 1B.

For GM3 microparticles 300 μL of heptane stored microparticles was added to 2 mL of solution A1 (10 mL of 10% glucose and 50 μL of extract from Example 1A). For GM1 microparticles 300 μL of heptane stored microparticles was added to 2 mL of solution A1 (10 mL of 10% glucose and 50 μL of extract from Example 1B). These mixtures were evaporated for 10 minutes using a rotary evaporator and transferred to a 2 ml tube. To ensure all microparticles were removed from the evaporation vessel, the vessel was rinsed with a solution comprising glucose+extract from Example 1A (for GM3 microparticles) or Example 1B (for GM1 microparticles) to bring the total volume back to 2 mL when added to the initial transferred volume.

9 Sprague-Dawley rats were fasted overnight. 8 rats were gavaged with 1.5 mL of the preparations outlined above. 1 rat was gavaged with 1.5 ml of 10% glucose containing 20 ug mCherry. 2 hours post-dosing the food was returned to the rats. After 4 hours, the rats were killed by slow fill CO₂ asphyxiation the sub-scapular fat was removed and a small piece of brown fat placed on a dish for confocal imaging and the rest snap frozen.

Small pieces of sub-scapular fat and liver were embedded in Tissue-Tek Optimum Cutting Temperature (OCT) Compound. Using a cryostat, 8 um sections were cut and placed on glass slides. These were placed at −20° C. for 48 hours to dry. Following this, slides were removed from the freezer and quickly stained with DAPI (0.1 μg/mL; Sigma product number D9542) in PBST before adding 2 drops of mounting media and placing a coverslip over the sections. These were left to dry at room temperature overnight then imaged using the confocal microscope. Images were quantitated using Image) software (National Institutes of Health, USA). The corrected total cryosection fluorescence (CTCF) was calculated using the formula: CTCF=Media of Integrated Density−(Media of Area of selected cell X Mean fluorescence of background readings).

FIG. 13D shows that the rat treated with the GM1 starch formulation has higher levels of fluorescence in the sub-scapular fat compared with the control GM1/starch rat or the rat treated with mCherry alone.

### vi. Studies of Transcytosis into Lymph Fluid

### Methods

Lymphatic pharmacokinetic studies: Male Sprague-Dawley rats with body weight 250-340 g were used in all studies. The rats were fed a standard diet prior to experiments. Each rat was fasted overnight with free access to water prior to surgical cannulation of the mesenteric lymph duct, carotid artery and duodenum in the morning.

Briefly, the rats were anaesthetized using isoflurane gas (1.5-5% according to response), placed on a heated pad at 37° C. and cannulas were inserted into the duodenum (for rehydration and dosing of microparticle formulations), mesenteric lymph duct (for lymph fluid collection) and carotid artery (for blood collection). Post-surgery, rats were re-hydrated for 0.5 h via intraduodenal infusion of normal saline at 1.5 mL/h prior to microparticle formulation administration.

Microparticle formulation administration: The microparticle formulations were administered into the duodenum via the cannula at a rate of 6 mL/h for 15 mins (1.5 mL total) or at 2 mL/h for 15 mins (0.5 mL total). After that, normal saline was infused into the duodenum at 1.5 mL/h for the remainder of the experiment to hydrate the animals.

Intravenous pharmacokinetic studies: Male Sprague-Dawley rats with body weight 250-340 g were used in all studies. The rats were fed a standard diet prior to experiments. On the day of dosing the rats were anaesthetized using isoflurane gas (1.5-5% according to response), placed on a heated pad at 37° C. and cannulas were inserted into the jugular vein (to enable intravenous injection) and carotid artery (to enable blood collection). After surgery the rats were allowed to rehydrate for 0.5 h with an intravenous infusion of 1.5 ml/h normal saline via jugular vein. Following the rehydration period, rats were administered trastuzumab (2 mg/kg) or peginterferon alpha-2a (5 μg/kg) via infusion of a 1 ml bolus over 1.5 min followed by saline infusion for 0.5 min into the jugular vein cannula. Following the dosing normal saline was infused intravenously at 1.5 ml/h for the remainder of the experiment. Whole blood (0.25 mL) was collected from the carotid artery cannula before dosing (blank) and at 0.5, 1, 2, 3, 4 5, 6, 7, and 8 hour into heparinised (5 μL of 1000 IU/ml heparin) eppendorf tubes and centrifuged at 3,500 g for 5 min to obtain plasma. Plasma samples were stored at −20° C. prior to analysis as described below.

Lymph sample collection: Lymph was continuously collected for 8 h into pre-weighed Eppendorf tubes containing 20 μL of 1,000 IU/mL heparin as anti-coagulant. The collection tubes were changed every 15 mins, 30 mins, 1 h or 2 hs. Lymph flow rate was measured by weight (i.e. gravimetrically).

Plasma sample collection: 400 μL of blood was collected at 0 h (before dosing) and at 1, 2, 3, 4, and 8 h after initiation of drug dosing into 1.5 mL Eppendorf tubes containing 5 μL of 1,000 IU/mL heparin as an anti-coagulant every 15 mins, 30 mins, 1 h or 2 hs. The blood samples were then centrifuged at 3500 g for 5 min and the clear supernatant plasma was aliquoted into new 1.5 mL Eppendorf tubes.

Lymph and plasma concentrations of trastuzumab and peginterferon alfa-2a were measured by ELISA assay using the ELISA kits for quantification of IgG (MabTech, VIC, Australia) as described previously (Chan et al., 2015; Dahlberg et al., 2014; Kaminskas et al., 2014). Samples were analysed in accordance with the manufacturer's instructions for the kits.

Lymph and plasma concentrations of adalimumab were measured by ELISA assay using the commercially available ELISA kit (Affymetrix eBioscience, San Diego, USA) for quantification of adalimumab, according to the manufacturer's instruction.

Lymph and plasma concentrations of R-phycoerythrin (R-PE) were measured by fluorescence assay using an EnSpire™ Plate Reader (Perkin Elmer, Perth, Australia) for quantification of R-PE (at 560 nm).

Formulation analysis: Formulations were centrifuged in a pre-weighed tube at 13,000 rpm for 60 minutes at 4° C. The supernatant was removed into another tube and the volume recorded. The weight of the particle pellet was recorded. Both the particle pellet and supernatant were stored at −20° C. For analysis, the particle pellet was re-suspended in the required volume of 20 mM sodium phosphate, pH 6.9 buffer. Both suspended particles and supernatant were diluted in sodium phosphate buffer for analysis by ELISA or fluorescent plate reader to measure the concentration of therapeutic agent or fluorescent protein in the pellet and supernatant fraction of the formulation.

Assay validation: The assays were validated for the concentration range 2-300 ng/ml (trastuzumab IgG), 0.4-25 ng/mL (adalimumab), and 0.01-10 μg/mL (R-PE). Linear standard curves were used for either the 5 lowest concentrations (for measurement of concentration in lymph and plasma samples) or the 5 highest concentrations (for measurement of concentration in formulation samples). Using this method the accuracy and precision of the assay were found to be within ±10% of expected with few exceptions at the lowest limit of quantitation (LLOQ) (at ±15-20%).

Lymph uptake calculations: Mass transport of the therapeutic agent or fluorescent protein into lymph fluid was calculated from the volume of lymph fluid collected and the measured concentrations of therapeutic agent or fluorescent protein in collected lymph samples. The % of the dose transported in lymph can be calculated from the ratio of the mass transport of the therapeutic agent or fluorescent protein in lymph and the dose administered.

### Example D9—Transcytosis of Microparticles Encapsulating R-PE into Lymph Fluid

To assess the transcytosis of microparticles into lymph rat 5 was dosed with microparticles comprising starch and R-PE using the protocol described below.
Rat 5 weight: 262 g
   Formulation summary:
      3× starch formulations containing R-PE at t=0 hrs, 2 hrs, 4 hrs
   Formulation 1: (15% Ultrex, 400 μg R-PE, 6 ml buttermilk extract, mixing time 15 min, mixing time after extract addition 5 min, mixing time for crop 2, settling time 24 hours, rotavapor time 10 min) dosed at t=0 hrs (infused over 15 minutes)
      R-PE mass added at outset of formulation: 400 μg of R-PE in 1.5 ml starch
      Formulation volume dosed: 2.0 ml
   Formulation 2: (15% Ultrex, 400 μg R-PE, 6 ml G600 extract, mixing time 15 min, mixing time after extract addition 5 min, mixing time for crop 2, settling time 24 hours, rotavapor time 10 min) (dosed at t=2 hrs (infused over 15 minutes)
      R-PE mass added at outset of formulation: 400 μg of R-PE in 1.5 ml starch
      Formulation volume dosed: 2.0 ml
   Formulation 3: (2% Ultrasperse, 200 μg, 50 μl buttermilk extract in 150 ml ethylacetate, mixing time 40 min, settling time 40 min, the 1 urn particles present in this formulation are shown in FIG. 24) (dosed at t=4 hrs (infused over 15 minutes)
      R-PE mass added at outset of formulation: 200 μg of R-PE in 2.0 ml of 4% w/v starch solution (i.e. 80 mg starch)
      Formulation volume dosed: 2.0 ml
   Uptake of R-PE in the lymphatic fluid was observed in Rat 5, as measured by fluorescence assay using an EnSpire™ Plate Reader (Perkin Elmer, Perth, Australia) for quantification of R-PE (at 560 nm).

### Example D10—Transcytosis of Microparticles Encapsulating Adalimumab into Lymph Fluid

To assess the transcytosis of microparticles into lymph fluid rat 7 was dosed with microparticles comprising starch and adalimumab (148 kDa monoclonal antibody) using the protocol described below.
Rat 7 weight: 312 g
   Formulation summary:
      2× starch formulations containing adalimumab dosed at t=0 hrs and t=2 hrs
   Formulation 1: (adalimumab, buttermilk extract) dosed at t=0 hrs (infused over 15 minutes)
      Adalimumab mass added at outset of formulation: 44.5 μl of adalimumab solution (containing 2.225 mg adalimumab) in 1.5 ml starch
      Formulation volume dosed: 1.5 ml
   Formulation 2: (adalimumab, G600 extract) dosed at t=2 hrs (infused over 15 minutes)
      Adalimumab mass added at outset of formulation: 44.5 μl of adalimumab solution (containing 2.225 mg Adalimumab) in 1.5 ml starch
      Formulation volume dosed: 1.5 ml
   Uptake of adalimumab in the lymphatic fluid was observed in Rat 7, as measured by an adalimumab Elisa kit in accordance with the manufacturer's instructions (Affymetrix eBioscience, San Diego, USA).
   Uptake of adalimumab in the plasma was observed in Rat 7, as measured by an adalimumab Elisa kit in accordance with the manufacturer's instructions (Affymetrix eBioscience, San Diego, USA).

### Example D11—Transcytosis of Microparticles Encapsulating Trastuzumab into Lymph Fluid

To assess the transcytosis of microparticles into lymph fluid rats 10 and 11 were dosed with microparticles comprising starch and trastuzumab (146 kDa monoclonal antibody) using the protocol described below.
Rat 10 weight: 265 g
   Formulation summary:
      1× starch formulation containing trastuzumab dosed at t=0 hrs, and 1× starch formulation containing peginterferon alpha-2a dosed at t=2 hrs
   Formulation 1: (1.5 mL, 15% Ultrex starch, trastuzumab 1 mg, G600 600 μL, G600 ethanol extraction process) dosed at t=0 hrs (infused for 15 minutes)
      Trastuzumab mass added at outset of formulation: 47.6 μl of 21 mg/ml stock solution added (containing a total of 1.0 mg trastuzumab) in 1.5 ml starch
      Formulation volume dosed: 1.5 ml
   Uptake of trastuzumab in the lymphatic fluid was observed in rat 10, as measured by a trastuzumab Elisa kit in accordance with the manufacturer's instructions (MabTech, VIC, Australia).
Rat 11 weight: 279 g
   Formulation summary:
      1× starch formulation containing peginterferon alpha-2a dosed at t=0 hrs, and 1× starch formulation containing trastuzumab dosed at t=2 hrs
   Formulation 1: (2% Ultratex starch, trastuzumab 1 mg) dosed at t=2 hrs (infused over 15 minutes)
      Trastuzumab mass added at outset of formulation: 47.6 μl of 21 mg/ml stock solution added (containing a total of 1.0 mg trastuzumab) in 4.0 ml of 2% starch solution (i.e. 80 mg starch)
      Formulation volume dosed: 1.5 ml
   Uptake of trastuzumab in the lymphatic fluid was observed in rat 11, as measured by a trastuzumab Elisa kit in accordance with the manufacturer's instructions (MabTech, VIC, Australia).

### E. Characterisation of Formulations

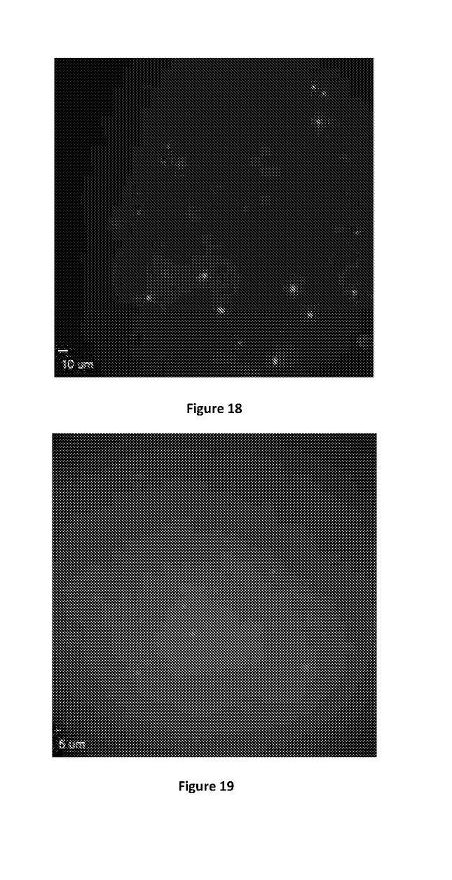

A microparticle formulation was prepared as described using the extract described in Example A1 using the process described in Example B15 and characterised using microscopy. Samples of the microparticles stored in heptane 2×50 μL were taken and dried in the nitrogen blow down dry evaporator. The mass of material remaining after removing the nitrogen was 4.67 mg for sample 1 and 11.54 mg for sample 2 suggesting a small amount of material was sampled. Microparticles were imaged on an Olympus IX83 deconvolution microscope. As shown in FIG. 18 microparticles of 5-10 microns were observed.

The above microparticle formulation was optimised by the addition of isopropanol to the heptane during mixing in the Silverson and the volume of ganglioside extract added. Isopropanol (8 ml) was added to the formulation while mixing in heptane and before the addition of the lipid extract. The microparticles were prepared using the following ingredients: 15% Ultrasperse starch batch, 3×1.5 ml Ultrasperse starch, R-PE 150 μg, Extract A1, particles were resuspended in a 10% glucose solution for imaging. Microparticles were imaged on an Olympus IX83 deconvolution microscope. As shown in FIG. 19 microparticles below 5 microns were observed.

This application claims priority from Australian Provisional Application No. 2016901677 entitled “Agent delivery system” filed on 6 May 2016, the entire contents of that application are hereby incorporated by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

The steps, features, integers, compositions and/or compounds disclosed herein or indicated in the specification of this application individually or collectively, and any and all combinations of two or more of said steps or features.

### REFERENCES

L. J. Chan, J. B. Bulitta, D. B. Ascher, J. M. Haynes, V. M. McLeod, C. J. Porter, C. C. Williams, L. M. Kaminskas, Mol Pharm, 12 (2015) 794-809.
A. M. Dahlberg, L. M. Kaminskas, A. Smith, J. A. Nicolazzo, C. J. Porter, J. B. Bulitta, M. P. McIntosh, Mol Pharm, 11 (2014) 496-504.
L. M. Kaminskas, D. B. Ascher, V. M. McLeod, M. J. Herold, C. P. Le, E. K. Sloan, C. J. Porter, J Control Release, 168 (2013) 200-208.

1. An agent preparation encapsulated within a membrane composition, wherein the agent preparation comprises an agent, and the membrane composition comprises a lipid and ganglioside, the ganglioside having a lipophilic domain and a hydrophilic domain, wherein the hydrophilic domain comprises (i) a mono-, di-, tri-, or tetra-saccharide residue, and (ii) one to four sialic acid residues linked to the saccharide residue, the membrane composition having an outer surface, wherein at least a portion of the hydrophilic domain is present on the outer surface.

2. The agent preparation encapsulated within the membrane composition of claim 1, which exists as a plurality of individual particles wherein each particle contains agent preparation individually encapsulated in membrane composition.

3. The agent preparation encapsulated within the membrane composition of claim 2, wherein the particles have a diameter ranging from about 0.01 to about 20 μm.

4. The agent preparation encapsulated within the membrane composition of claim 2, wherein the particles have a diameter ranging from about 0.05 to about 5 μm.

5. The agent preparation encapsulated within the membrane composition of claim 2, wherein the particles are nanoparticles or microparticles.

6. The agent preparation encapsulated within the membrane composition of any one of claims 1 to 5, wherein the amount of ganglioside in the membrane composition ranges from about 0.01 to about 20% of the total weight of the ganglioside and lipid.

7. The agent preparation encapsulated within the membrane composition of any one of claims 1 to 6, wherein the amount of ganglioside in the membrane composition ranges from about 0.02 to about 5% of the total weight of the ganglioside and lipid.

8. The agent preparation encapsulated within the membrane composition of any one of claims 1 to 7, wherein the lipid is at least one of a sphingolipid, sterol lipid, phospholipid, and glycerol lipid.

9. The agent preparation encapsulated within the membrane composition of claim 8, wherein the lipid is at least one of a sphingosine, ceramide, sphingomyelin, and cholesterol.

10. The agent preparation encapsulated within the membrane composition of any one of claims 1 to 9, wherein the agent is a therapeutic agent, diagnostic agent, nutritional agent, cosmetic agent, food or food ingredient, or combination thereof.

11. The agent preparation encapsulated within the membrane composition of any one of claims 1 to 10, wherein the agent is a therapeutic agent having a molecular weight ranging from about 1 kDa to about 300 kDa.

12. The agent preparation encapsulated within the membrane composition of any one of claims 1 to 11, wherein the agent is a therapeutic agent having a molecular weight ranging from about 50 Da to about 1 kDa.

13. The agent preparation encapsulated within the membrane composition of any one of claims 1 to 12, wherein the agent preparation further comprises an excipient.

14. The agent preparation encapsulated within the membrane composition of any one of claims 1 to 12, wherein the agent preparation comprises a vesicular structure.

15. The agent preparation encapsulated within the membrane composition of claim 13, wherein the excipient is a gelling agent.

16. The agent preparation encapsulated within the membrane composition of claim 15, wherein the gelling agent is a pH sensitive or heat sensitive gelling agent.

17. The agent preparation encapsulated within the membrane composition of claim 15, wherein the gelling agent comprises or consists of pectin or modified pectin.

18. The agent preparation encapsulated within the membrane composition of any one of claims 1 to 17, wherein the agent or agent preparation has a hydrophilic-lipophilic balance (HLB) of between about 10 and 20.

19. The agent preparation encapsulated within the membrane composition of claim 18, wherein the membrane composition encapsulates the agent preparation with a first bilayer comprising a lipid and ganglioside, the ganglioside having a lipophilic domain and a hydrophilic domain, wherein the hydrophilic domain comprises (i) a mono-, di-, tri-, or tetra-saccharide residue, and (ii) one to four sialic acid residues linked to the saccharide residue, the bilayer having an outer surface, wherein at least a portion of the hydrophilic domain of the ganglioside is present on the outer surface.

20. The agent preparation encapsulated within the membrane composition of claim 19, wherein the membrane composition comprises at least one further bilayer that encapsulates the first bilayer, and wherein the outermost bilayer has an outer surface and at least a portion of the hydrophilic domain of the ganglioside in the outermost bilayer is present on the outer surface.

21. The agent preparation encapsulated within the membrane composition of any one of claims 1 to 17, wherein the agent or agent preparation has a hydrophilic-lipophilic balance (HLB) of between about 0 and 10.

22. The agent preparation encapsulated within the membrane composition of claim 20, wherein the membrane composition encapsulates the agent preparation with a first monolayer comprising a lipid and ganglioside, the ganglioside having a lipophilic domain and a hydrophilic domain, wherein the hydrophilic domain comprises (i) a mono-, di-, tri-, or tetra-saccharide residue, and (ii) one to four sialic acid residues linked to the saccharide residue, the monolayer having an outer surface, wherein at least a portion of the hydrophilic domain of the ganglioside is present on the outer surface.

23. The agent preparation encapsulated within the membrane composition of claim 22, wherein the membrane composition comprises at least one further bilayer that encapsulates the first monolayer, and wherein the outermost bilayer has an outer surface and at least a portion of the hydrophilic domain of the ganglioside in the outermost bilayer is present on the outer surface.

24. A pharmaceutical composition comprising the agent preparation encapsulated within the membrane composition according to any one of claims 1 to 23 and a pharmaceutically acceptable excipient.

25. The composition of claim 24, wherein the composition is formulated for enteral or parenteral administration.

26. The composition of claim 24, wherein the composition is formulated for oral administration.

27. The composition of claim 24, wherein the composition is formulated for injectable or transdermal administration.

28. An agent delivery system comprising the agent preparation encapsulated within a membrane composition of any one of claims 1 to 23 or the pharmaceutical composition of any one of claims 24 to 27.

29. The agent delivery system of claim 28 for delivering an agent to or via the lymphatic system of a subject wherein the agent preparation encapsulated within the membrane composition is internalized into the subject by transcytosis across the subject's gastrointestinal epithelial barrier.

30. The agent delivery system of claim 28 or claim 29 for delivering an agent to a predetermined cell, tissue or organ in a subject by having present a portion of the hydrophilic domain of a species of ganglioside on the outer surface of the membrane composition.

31. A method for making a membrane composition comprising a lipid and ganglioside, wherein the method comprises the steps of:
(a) contacting a membrane composition source comprising a lipid and ganglioside with an alcohol solution to provide a liquid mixture comprising insoluble solids;
(b) separating the insoluble solids from the liquid mixture to obtain an alcohol solution comprising a lipid and ganglioside; and
(c) removing at least some of the alcohol from the alcohol solution to obtain the membrane composition.

32. The method of claim 31, wherein the alcohol solution is isopropanol.

33. A method for making an agent preparation encapsulated within a membrane composition, wherein the agent preparation comprises an agent and the membrane composition comprises a lipid and ganglioside, the ganglioside having a lipophilic domain and a hydrophilic domain, wherein the hydrophilic domain comprises (i) a mono-, di-, tri-, or tetra-saccharide residue, and (ii) one to four sialic acid residues linked to the saccharide residue, and wherein the method comprises the steps of:
a) combining the agent preparation with a liquid in which the agent preparation is immiscible or insoluble;
(b) subjecting the liquid and agent preparation to mixing effective to form particles of the agent preparation in the liquid; and
(c) adding the membrane composition to the agent preparation at a time during or after step (b) to encapsulate each particle of the agent preparation within the membrane composition wherein at least a portion of the hydrophilic domain is present on the outer surface.

34. The method of claim 33, wherein the agent preparation further comprises one or more excipients.

35. The method of claim 33 or claim 34, wherein at least 20% of the agent preparation from step (a) is encapsulated in step (c).

36. The method of any one of claims 33 to 35, wherein the agent preparation encapsulated within the membrane composition has a diameter ranging from about 0.1 to about 20 μm.

37. A method for delivering an agent to or via the lymphatic system of a subject comprising administering to the subject the agent preparation encapsulated within a membrane composition of any one of claims 1 to 23 or the pharmaceutical composition of claims 24 to 26, wherein the agent preparation encapsulated within the membrane composition is internalized into the subject by transcytosis across the subject's gastrointestinal epithelial barrier.

38. The method of claim 37, wherein the composition is administered orally.

39. The method of claim 37 or claim 38, wherein the agent is released from the agent preparation encapsulated within the membrane composition in the lymphatic system.

40. The method of any one of claims 37 to 39, wherein the agent is released from the agent preparation encapsulated within the membrane composition in the blood circulatory system.

41. A method for delivering an agent to a predetermined cell, tissue or organ in a subject comprising administering to the subject the agent preparation encapsulated within a membrane composition of any one of claims 1 to 23 or the pharmaceutical composition of claims 24 to 27.

42. A method for delivering an agent to a subject's neural cell, neural tissue or brain, comprising administering to the subject the agent preparation encapsulated within a membrane composition of any one of claims 1 to 23 or the pharmaceutical composition of claims 24 to 27.

43. A method for delivering an agent to a subject's adipocyte or adipose tissue, comprising administering to the subject the agent preparation encapsulated within a membrane composition of any one of claims 1 to 23 or the pharmaceutical composition of claims 24 to 27.

44. A method for delivering an agent to a subject's renal cell or kidney, comprising administering to the subject the agent preparation encapsulated within a membrane composition of any one of claims 1 to 23 or the pharmaceutical composition of claims 24 to 27.

45. A method for treating a disease or disorder, comprising administering to a subject in need thereof (i) an effective amount of the agent preparation encapsulated within a membrane composition of any one of claims 1 to 23 or the pharmaceutical composition of any one of claims 24 to 27.

46. A membrane composition comprising a ganglioside and lipid, the ganglioside having a lipophilic domain and a hydrophilic domain, wherein the hydrophilic domain comprises (i) a mono-, di-, tri-, or tetra-saccharide residue, and (ii) one to four sialic acid residues linked to the saccharide residue, wherein the amount of the ganglioside is between about 0.01 to about 20% based on the total weight of the ganglioside and lipid.

47. Use of a membrane composition for encapsulating an agent preparation comprising an agent and optionally one or more excipients, wherein the membrane composition comprises a lipid and ganglioside, the ganglioside having a lipophilic domain and a hydrophilic domain, wherein the hydrophilic domain comprises (i) a mono-, di-, tri-, or tetra-saccharide residue, and (ii) one to four sialic acid residues linked to the saccharide residue.
